(19) United States
(12) Patent Application Publication
(10) Pub. No.: US 2021/0361177 A1
(43) Pub. Date: Nov. 25, 2021

(54) Non-Invasive Continuous Blood Pressure Monitoring

(71) Applicant: HUMA THERAPEUTICS LIMITED, London (GB)

(72) Inventors: Nitagauri SHAH, Welwyn Garden City, Hertfordshire (GB); Sandeep SHAH, Welwyn Garden City, Hertfordshire (GB); Mohammed MUKHRAMUDDIN, Welwyn Garden City, Hertfordshire (GB); Peter PARNELL, Welwyn Garden City, Hertfordshire (GB); Pradeep JOOLURI, Welwyn Garden City, Hertfordshire (GB)

(73) Assignee: HUMA THERAPEUTICS LIMITED, London (GB)

(21) Appl. No.: 16/968,133

(22) PCT Filed: Feb. 6, 2019
(86) PCT No.: PCT/IB2019/050972

§ 371 Date: Aug. 6, 2020

Related U.S. Application Data
(60) Provisional application No. 62/628,072, filed on Feb. 8, 2018.
(60) Provisional application No. 62/628,174, filed on Feb. 8, 2018.
(60) Provisional application No. 62/627,120, filed on Feb. 6, 2018.

(51) Int. Cl. A61B 5/021; A61B 5/00; A61B 5/022

(52) U.S. Cl. CPC A61B 5/02116; A61B 5/681; A61B 2562/028; A61B 2562/0233; A61B 5/022

(57) ABSTRACT

Non-invasive blood pressure monitoring systems and methods provide continuous, “beat-to-beat” measures of blood pressure without the need for an inflatable cuff, and/or without the need for calibration of the system or method for a particular subject using a separate blood pressure measurement system. Embodiments include various wrist-worn blood pressure monitoring devices adapted to be worn on the wrist comfortably and obtain a blood pressure measurement of the radial artery that traverses the wrist. Other implementations are adapted to measure blood pressure in a variety of other blood vessels in the body, such as the carotid artery and the templar artery, to name just two of many examples. This document describes additional designs of micro-motion sensing systems for use in such non-invasive blood pressure monitoring systems and methods.

### RELATED APPLICATIONS

This document relates to, and claims priority to, the following commonly assigned Provisional Patent Application Serial Nos.: 62/627,120, filed Feb. 6, 2018 to Nitagauri Shah et al., entitled “Non-Invasive Continuous Blood Pressure Monitoring” (the “'120 provisional patent application”); 62/628,072, filed Feb. 8, 2018 to Nitagauri Shah et al., entitled “Wrist-Worn Non-Invasive Continuous Blood Pressure Monitoring Device” (the “'072 provisional patent application”); and 62/628,174, filed Feb. 8, 2018 to David Pearce et al., entitled “Mobile Program Application for Non-Invasive Continuous Blood Pressure Monitoring (the “'174 provisional patent application”). The content of the '120, '072, and '174 provisional patent applications is incorporated by reference into this document.

### TECHNICAL FIELD

This document relates to non-invasive blood pressure monitoring systems and methods that provide continuous, “beat-to-beat” measures of blood pressure without the need for an inflatable cuff or calibration.

### BACKGROUND

Non-invasive measurement of blood pressure has commonly been provided using cuff-based systems, which provide one set of blood pressure measurements of blood pressure (e.g., a systolic measure and a diastolic measure) for each inflation and deflation cycle of the inflatable cuff. Each inflation and deflation cycle spans multiple heartbeats, and cuff-based systems therefore provide only intermittent measures of blood pressure. In addition, cuff-based blood pressure measurement systems are uncomfortable to the subject whose blood pressure is being monitored, are inconvenient and bulky, and have been found to be generally subject to significant inaccuracies. Further yet, cuff-based systems require the interruption of normal blood flow, including occlusion of the artery, to take a blood pressure measurement.

Invasive blood pressure measurement systems exist but have significant disadvantages. For example, so-called “arterial line” systems involve a catheter being invasively introduced into the arterial system of a patient, typically at the wrist. Arterial line systems provide a continuous “beat-to-beat” measure of blood pressure, and are often used in an intensive care unit (“ICU”) setting where continuous “beat-to-beat” blood pressure monitoring is critical. Arterial line systems have the disadvantages of being costly in terms of the time and difficulty in terms of getting the arterial line in place in a patient, and come with the risk of infection owing to the invasive nature of the technology. In addition, arterial lines are typically removed from the patient in the ICU before the patient is sent to a recovery ward, despite recent studies supporting use of continuous blood pressure monitoring in the recovery ward to avoid serious post-procedure risks. As a consequence, a patient in the recovery ward is often subjected to blood pressure monitoring with a cuff-based system that periodically inflates and deflates to take a measurement, which is disruptive to recovery and in some cases is disengaged so the patient may sleep without interruption.

Various efforts have been made over the years to provide a workable non-invasive blood pressure monitoring solution that does not require an inflatable cuff and that provides a continuous measure of blood pressure. Achieving such a workable solution has proven to be extremely difficult. Improvement in the state of the art of blood pressure monitoring is greatly needed in all medical and consumer markets in which blood pressure monitoring devices may be used.

### SUMMARY

In various embodiments, the devices, systems, and methods disclosed in this document provide non-invasive, continuous, beat-to-beat measurements of blood pressure, without the need for an inflatable cuff or other blood vessel constricting device to obtain a blood pressure measure. Specifically, this document describes a health monitoring system that is adapted to, among other things, monitor blood pressure of a subject non-invasively and continuously, on a “beat-to-beat” basis, without the need for an inflatable cuff and without the need for calibration of the device for a particular subject using a separate blood pressure measurement device.

Embodiments described in this document include various wrist-worn blood pressure monitoring devices adapted to be worn on the wrist comfortably to obtain a blood pressure measurement of the radial artery that traverses the wrist. Other implementations of the beat-to-beat systems and methodology described in this document are adapted to measure blood pressure in a variety of other blood vessels in the body, such as the carotid artery and the templar artery, to name just two of many examples. Embodiments of body-worn or applied blood pressure monitoring devices additionally include patch-type devices that may be applied on various parts of the body, including at the wrist for monitoring the radial artery, on the upper arm at a location adjacent a suitable place to measure blood pressure at the brachial artery, on the neck at a region adjacent the carotid artery, and on the back at a region to measure blood pressure at the renal artery, etc. Other embodiments may include smart band devices adapted to be worn on the ventral side of the wrist and connectable to a smart band device, wherein the blood pressure sensing device structure may be included, in part, within the smart band device. Yet further embodiments may be probe type devices that may be manually applied against the surface of the skin adjacent an underlying artery.

This document also describes additional designs for a wrist-worn device for use with non-invasive blood pressure monitoring systems and methods that provide continuous, “beat-to-beat” measures of blood pressure without the need for an inflatable cuff, and without the need for calibration of the system or method for a particular subject using a separate blood pressure measurement system. Embodiments described in this document include various wrist-worn blood pressure monitoring devices adapted to be worn on the wrist comfortably to obtain blood pressure measurements of the radial artery that traverses the wrist. This document also describes designs for mobile device program applications for use with non-invasive blood pressure monitoring systems and methods that provide continuous, “beat-to-beat” measures of blood pressure without the need for an inflatable cuff, and without the need for calibration of the system or method for a particular subject using a separate blood pressure measurement system. Embodiments described in this document may, as an example, interact with wrist-worn blood pressure monitoring devices adapted to be worn on the wrist comfortably to obtain a blood pressure measurement of the radial artery that traverses the wrist, as well as other body worn or applied devices for monitoring blood pressure of other body vessels.

This document also describes additional designs of micro-motion sensing systems for use in non-invasive blood pressure monitoring systems and methods to provide continuous, “beat-to-beat” measures of blood pressure without the need for an inflatable cuff and without the need for calibration of the system or method for a particular subject using a separate blood pressure measurement system. Such micro-motion sensing systems, in these additional examples, utilize optical power modulation techniques for micro-motion sensing. The micro-motion sensing systems may be utilized in blood pressure monitoring devices adapted to be worn or applied to a skin surface of a subject, adjacent an underlying blood vessel, to obtain a blood pressure measurement.

In one aspect, this document provides a micro-motion sensing device that may provide for low-profile designs for continuous blood pressure monitoring, utilizing techniques of optical power modulation. Such a micro-motion sensing device includes a flexible circuit substrate; an optical waveguide provided at least in part on a first region of the flexible circuit substrate; and electronic circuitry provided on a second region of the flexible circuit substrate, wherein the second region is non-overlapping with the first region; and a skin interface component. The skin interfacing system has a skin-facing surface for positioning against a skin surface adjacent an underlying blood vessel, and an inner surface opposite the skin-facing surface positioned and configured to bear against at least one of a side surface of the optical waveguide and a surface of the first region of the flexible circuit substrate, to modulate optical power propagating through the optical waveguide. The first flexible substrate region and the second flexible substrate region are oriented such that, when the device is applied adjacent a skin surface, the first flexible substrate region and the second flexible substrate region overlie different non-overlapping regions of skin.

In various implementation, the device may include one or more of the following features. The first region of the flexible circuit substrate may be configured and positioned within the device to be permitted, during normal operation of the device, to flex in response to bearing forces applied by the inner surface of the skin interfacing system, whereas the second region of the flexible circuit substrate may be configured and positioned within the device such that, during normal operation of the device, the second region remains stationary.

The flexible circuit substrate may further include a third region that resides between, and is non-overlapping with, the first and second regions of the flexible circuit substrate. The third region of the flexible circuit substrate may have provided thereon a portion of the optical waveguide. The third region of the flexible circuit substrate may be configured and positioned within the device such that, during normal operation of the device, the third region also remains stationary.

The three-region flexible circuit substrate, when assembled in the device, may be configured in the shape of a “flattened Z.” In such a configuration, the first, third, and second regions of the flexible circuit substrate may correspond to, respectively, first, second, and third legs of the flattened Z shape. In other configurations, the flexible circuit substrate, when assembled in the device, may be configured in a generally planar shape.

In another aspect, this document provides a micro-motion sensing device that may, in some implementations, provide for an improved or eased ability to provide a device that is water resistant or waterproof. Such a micro-motion sensing device includes an optical waveguide; and a skin interface component comprising: (i) a button structure having a skin-facing surface for positioning against a skin surface adjacent an underlying blood vessel and an inner surface opposite the skin-facing surface positioned and configured to cause the optical waveguide to be flexed and/or compressed to modulate optical power propagating through the optical waveguide; and (ii) a coil spring structure provided under an upper portion of the button structure and encompassing a lower portion of the button structure. The coil spring structure may be configured to bias the button structure outward in the direction of the skin-facing surface.

In various implementations, the device may further include a housing having an opening formed therein. The skin interface component may be positioned to extend through the housing opening.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram of a system for determining blood pressure measures for a subject.

FIG. 2 is a diagram of a micro-motion sensor that may be used in the system of FIG. 1.

FIGS. 3A-B are diagrams illustrating the concept of “optical power modulation” (“OPM”), to illustrate one manner in which the micro-motion sensor of FIG. 2 may operate.

FIG. 4 is a block diagram of a body worn or applied monitoring device that may be used in the system of FIG. 1.

FIGS. 5A-C show flowcharts of the operation of blood pressure monitoring systems of the type shown in FIGS. 1-4.

FIG. 5D shows a graph of a continuous motion waveform and measurements thereof.

FIGS. 6A-Q3 illustrate an embodiment of a wrist worn monitoring device.

FIGS. 7A-I provide further illustration of an embodiment of a wrist-worn monitoring device similar to that shown in FIGS. 6A-Q3, illustrating an “across the wrist” (i.e., along the radial artery) layout of the micro-motion sensor in the device.

FIGS. 8A-I illustrate another embodiment of a wrist-worn monitoring device, illustrating an “along the wrist” (i.e., across the radial artery) layout of the micro-motion sensor in the device.

FIGS. 9A-B illustrate yet another embodiment of a wrist-worn monitoring device having a wired connection to a dedicated control and display device.

FIGS. 10A-E are diagrams of an embodiment of a micro-motion sensing system having a micro-motion sensor device in a configuration that may be referred to as a “Z” configuration.

FIGS. 11A-E are diagrams of another embodiment of a micro-motion sensing system having a micro-motion sensor device in a configuration that may be referred to as a “straight” or “flattened” configuration.

FIGS. 12A-F are diagrams of another embodiment of a micro-motion sensing system utilizing a coil spring to provide for the biasing of a button or pad structure to a rest position.

FIG. 13 is a diagram showing an embodiment of a wrist-worn blood pressure monitoring device being worn on the wrist of a human subject, and a general purpose local device in the form of a smartphone having a blood pressure monitoring application program provided thereon.

FIG. 14 is a perspective diagram of the wrist-worn blood pressure monitoring device shown in FIG. 13.

FIG. 15 is a perspective diagram of an embodiment of a wrist-worn blood pressure monitoring device similar to the embodiment of FIGS. 13-14 yet in a different color scheme.

FIGS. 16A-B are two different perspective diagrams of another embodiment of a wrist-worn blood pressure monitoring device.

FIGS. 17A-B are two different perspective diagrams of another embodiment of a wrist-worn blood pressure monitoring device similar to the embodiment of FIGS. 16A-B yet in a different color scheme and a different design for a side portion outer plate.

FIGS. 18A-C are three different perspective diagrams of another embodiment of a wrist-worn blood pressure monitoring device.

FIGS. 19A-C are three different perspective diagrams of another embodiment of a wrist-worn blood pressure monitoring device similar to the embodiment of FIGS. 18A-C yet in a different color scheme and a different design for a side portion outer plate.

FIG. 20 is a diagram showing an embodiment of a wrist-worn blood pressure monitoring device being worn on the wrist of a human subject, and a general purpose local device in the form of a smartphone having a blood pressure monitoring application program provided thereon.

FIGS. 21A-B are two parts of a flowchart describing the operation of a smartphone program application used in connection with a blood pressure monitoring device.

FIGS. 22A-J show an embodiment of a series of screen snapshots generated by a smartphone program application used in connection with a blood pressure monitoring device.

FIG. 23 is a block diagram of computing devices that may be used to implement the systems and methods described in this document, as either a client or as a server or plurality of servers.

Like reference symbols in the various drawings indicate like elements.

### DETAILED DESCRIPTION

This document relates to monitoring health information of a subject such as a person or animal, particularly but not exclusively, blood pressure information. In various embodiments, the devices, systems, and methods disclosed herein provide non-invasive, continuous, and “beat-to-beat” measurements of blood pressure, without the need for an inflatable cuff or other blood vessel constricting device to obtain a blood pressure measure, and without the need for calibrating the devices, systems, and methods for a particular subject to another blood pressure measurement device.

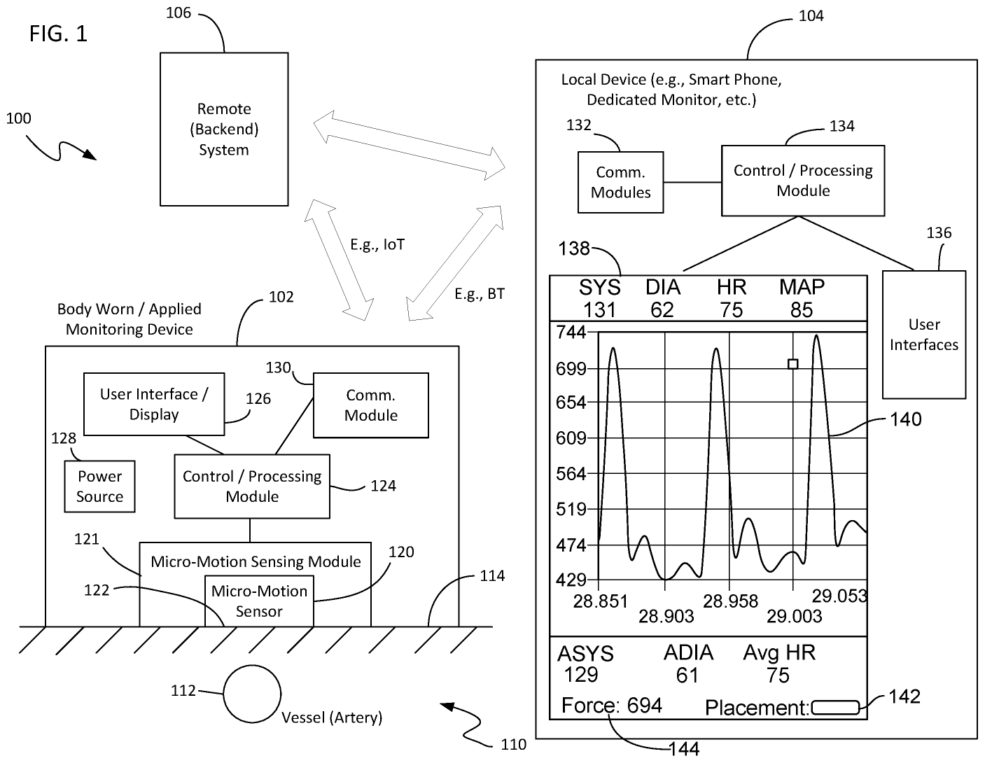

FIG. 1 shows generally a health monitoring system 100 that is adapted to, among other things, monitor blood pressure of a subject non-invasively and continuously, on a “beat-to-beat” basis, without the need for an inflatable cuff and without the need for calibration of the device for a particular subject using a separate blood pressure measurement device. The monitoring system 100 shown in FIG. 1 includes a body worn or applied monitoring device (“W/AD”) 102, a local device 104 in communication with the monitoring device 102, and a remote or back-end system 106 in communication with the local device 104, the monitoring device 102, or both. The body worn or applied monitoring device 102 may be applied to or worn against the skin 114 of a body part 110 in the vicinity of a body blood vessel 112 such as an artery, such that the blood vessel 112 is generally adjacent the skin location where the monitoring device 102 is worn or applied. For example, the monitoring device 102 may be configured as a wrist-worn device, to monitor blood pressure in the radial artery, which is an artery extending from the arm, and across the wrist, to carry oxygenated blood to the hand. In other implementations, the monitoring device 102 may be a patch or probe type device applied against the skin of a person adjacent other body arteries such as the carotid artery. Monitoring blood pressure in the carotid artery is particularly useful in assessing cardiovascular health.

An example of the body worn or applied monitoring device 102 is shown in FIG. 1 in simplified form, excluding various optional components that may be included. As shown in FIG. 1, the monitoring device 102 includes a skin surface micro-motion sensor 120. The sensor 120 may, for example as illustrated, be included in a micro-motion sensing module 121 having other components in addition to the motion transducing components of the sensor 120. The micro-motion sensor 120 is configured and positioned in the monitoring device 102 so that an outer surface 122 of the micro-motion sensor 120 may be applied directly to a surface of the skin 114 generally adjacent a body vessel 112. In general, the micro-motion sensor 120, when applied against the skin with only a relatively small amount of comfortably tolerated hold-down force, is able to sense movement on the surface of the skin created by the pulsing of blood through the underlying blood vessel 112. For example, the sensor may be applied against the skin adjacent the artery with a hold-down force sufficient to hold the sensor 120 against the skin but not so great that the hold-down pressure constricts the underlying blood vessel 112 (e.g., in a range of about 5-15 mm Hg or other similar range as discussed below, which is able to be comfortably tolerated by a subject undergoing blood pressure monitoring). In various embodiments as will be discussed in detail below, the micro-motion sensor 120 may be a highly sensitive opto-mechanical sensor that translates miniscule skin surface movements into a sensor output signal that is indicative of skin surface micro-movements related to the flow of blood through an underlying blood vessel.

The body worn or applied monitoring device 102 may also include a control and processing module 124 that controls operation of the micro-motion sensing module 121 and receives and processes the continuous sensor micro-motion output signal produced by the micro-motion sensor 120. The output signal of the micro-motion sensor 120 may, for example, be an electrical signal created by an optical detector within the sensor 120, in the case for example of the micro-motion sensor 120 being of an opto-electronic type as mentioned above. The processing module 124 may, for example, filter the electrical signal, perform an analog-to-digital conversion of the electrical signal, and perform mathematical and other processing operations on the electrical signal to generate (1) a digitized display of the filtered and digitized output of the sensor 120 corresponding to the continually changing blood pressure in the underlying artery adjacent the sensor 120 (and/or may generate a blood pressure waveform generated from the output of the sensor 120, which may be referred to as an arteriogram); and/or (2) blood pressure parameters (e.g., systolic blood pressure, diastolic blood pressure, mean arterial pressure, pulse pressure, cardiac output, etc.) for each cardiac cycle of the heart (in other words, “beat-to-beat” continuous measures of blood pressure and related biometric measures). The monitoring device 102 may also include buffer and/or longer-term memory, not shown in FIG. 1, to store digitized sensor and/or blood pressure waveform data and/or data representative of blood pressure measures and related biometric data on a beat-to-beat, average, or other basis.

The monitoring device 102 may include additional components as required or desired. As shown in FIG. 1, the monitoring device 102 may include various user interface components 126, such as user input devices and output devices (e.g., various indicators and/or visual displays). One example output provided by the monitoring device 102 may be a continually updated waveform showing a display of the filtered and digitized output of the sensor 120 (or a continuous, beat-to-beat measure of blood pressure that is generated based off the output of the sensor 120), along with or alternatively, various displayed blood pressure and other related biometric measures (e.g., systolic blood pressure, diastolic blood pressure, mean arterial pressure, pulse pressure, cardiac output, etc.) that may be updated for each cardiac cycle (in other words, “beat-to-beat” measures, with the system providing at least some of these measurements for each cardiac cycle) and/or may be provided in the form of average measures over a period of, for example, ten (10) cardiac cycles. The monitoring device 102 may also include a power source 128, which in various implementations may take the form of a self-contained battery power source, connection circuitry and/or leads to an external direct current (“DC”) source of power, and/or power conversion circuitry to convert an external alternating current (“A/C”) source of power to DC power.

The monitoring device 102 may also include one or more communication modules 130 to enable communications with external equipment, such as the local device 104, which may be for example a smartphone device and/or a dedicated monitoring device, and/or the remote or back-end system 106 (e.g., a cloud-based system). The communication module 130 may be adapted to perform wireless or wired communication to an external device or system. The communication module 130 may enable, for example, the transfer of continuously generated waveform data and related biometric information to the external equipment, either continuously as the sensor and/or blood pressure waveform and related biometric information is being generated, or as an upload of information generated and temporarily stored in the monitoring device 102. The communication module 130 may also enable the receipt of commands and information from external equipment and/or the transfer of various other information to the external equipment (e.g., low battery and other state condition information, etc.). In the case of wireless communication, the communication module 130 may enable communications via a Bluetooth®, including Bluetooth® Low Energy (“BTLE”), Wi-Fi, cellular, various Internet-of-Things communication techniques, or other similar or suitable communications methods.

The local device 104 may be, for example, a general-purpose smartphone device having a specially designed application program (“App”), and alternatively or additionally may be a special-purpose, or in other words a “dedicated,” medical monitoring device. The local device 104 may be considered local in that it is adapted to be co-located in the same vicinity with the subject being monitored by the monitoring device 102. The local device 104 may include, for example, display capability to enable the continuous, beat-to-beat display of blood pressure measures and other related monitored and/or calculated biometric information. The local device 104 has a communication module 132 to enable communications with the monitoring device 102 and with the remote or back-end system 106. Communications with the monitoring device 102 may be done wirelessly, using for example, Bluetooth® communications circuitry and protocol as mentioned above, or any other acceptable low energy wireless communications systems and protocols. Communications with the remote system 106 may be accomplished using various wired and wireless networks, employing appropriate security standards given the personal nature of the data being transmitted. The local device 104 also has a control and processing module 134 to perform the control and processing functions required of the local device.

The local device 104 also may have user interface components 136 such as user input mechanisms, as well as output mechanisms and a visual display 138, on which beat-to-beat representations of blood pressure information and/or other biometric information may be displayed. As with the example shown in FIG. 1, the visual display 138 may include a continuous waveform 140 of the filtered and digitized output of the sensor 120 presented in a graph showing amplitude of the signal with respect to time. Alternatively, the visual display 138 may show a continuous waveform of measured blood pressure (arteriogram) that is generated from the output of the sensor 120. The visual display 138 in FIG. 1 shows a continuous waveform 140 for slightly less than three full cardiac cycles, there being three waveform peaks shown on the display 138 that correspond to blood pressure systolic peaks (the highest measure of a cycle). A systolic peak corresponds to the heart's blood pumping action.

In addition, the visual display 138 may include beat-by-beat measures, which in FIG. 1 are provided along the top of the display 138. Those beat-to-beat measures in the example of FIG. 1 include systolic blood pressure (“SYS” of 131 mm Hg), diastolic blood pressure (“DIA” of 62 mm Hg), heart rate (“HR” of 75 beats per minute), and mean arterial pressure (“MAP” of 85 mm Hg). Additional beat-to-beat measures may be provided in other implementations. Mean arterial pressure (“MAP”) may be calculated in a number of different ways; some example ways in which MAP may be calculated is from the formula of MAP=DIA+⅓ (SYS−DIA), and various other MAP calculation or determination methods.

The visual display 138 may also include average measures of blood pressure and/or other biometric data, which in the FIG. 1 example is provided near the bottom of the display 138. These average measures may be averages for a defined number of cardiac cycles, for example, ten (10) consecutive cardiac cycles. The average measures in the FIG. 1 example are average systolic blood pressure (“ASYS” of 129 mm Hg), average diastolic blood pressure (“ADIA” of 61 mm Hg), and average heart rate (“Avg HR” of 75 beats per minute).

The display 138 may also provide a “placement” indication with a bar 142 that may be color coded (e.g., green or red) to indicate whether conditions are proper for a blood pressure measurement to be made. As an example, the indicator 142 may indicate to a user whether or not any or all of the following conditions are present: (1) the micro-motion sensor 120 is placed with an appropriate amount of hold-down force against the skin; (2) the micro-motion sensor 120 is placed in a proper location on the surface of the skin vis-à-vis an underlying artery; and (3) conditions are proper for a diagnostically useful blood pressure measurement to be taken, which may take into account various other biometric sensing devices such as activity sensors, position sensors, temperature sensors, ECG sensors, etc., if available.

Specifically with respect to determining whether the conditions for blood pressure measurement are appropriate for the blood pressure measurement to be useful, such a determination may include a determination of whether the subject has been at rest for a specified period of time, whether the monitoring device 102 is positioned at a proper level in relation to the level of the heart of the subject, and other conditions that are set by standards organizations to define the conditions for a diagnostically useful blood pressure measurement. In some implementations, the monitoring device 102 may provide blood pressure information under conditions other than conditions that are ideal for diagnostically useful conditions. For example, it may be desirable to measure blood pressure under certain active subject states. Alternatively or additionally, in some implementations the monitor 102 or external equipment may be configured to receive measured blood pressure information taken during the course of various sensed conditions (e.g., when the subject is active, when the monitoring device is not positioned at heart level, etc.) and may transform that information into blood pressure information that is meaningful diagnostically.

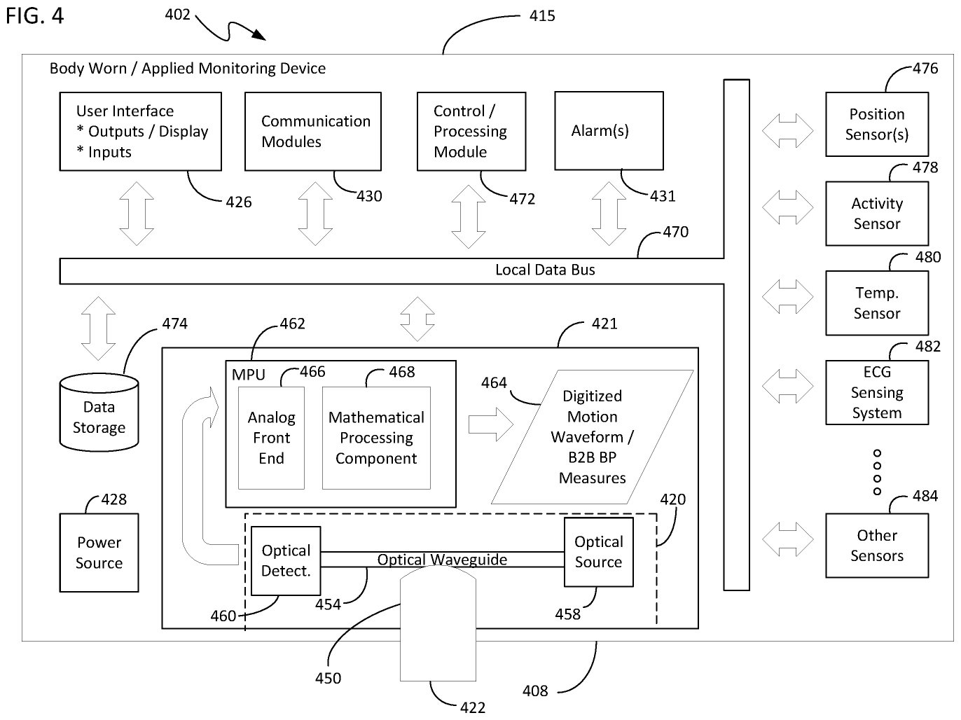

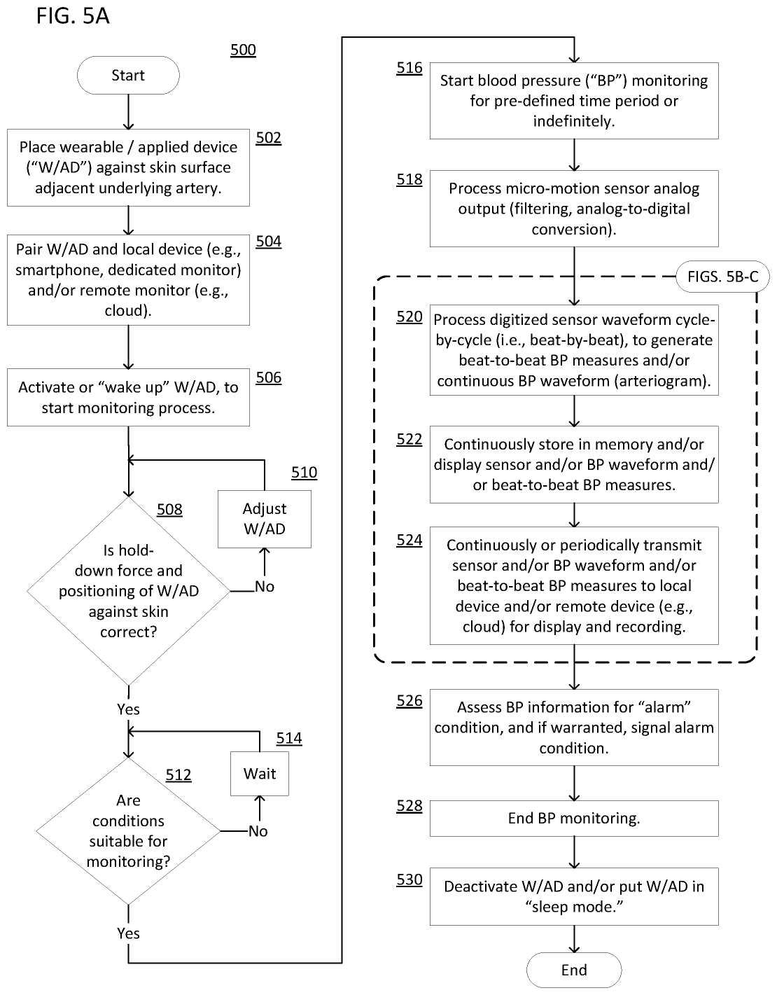

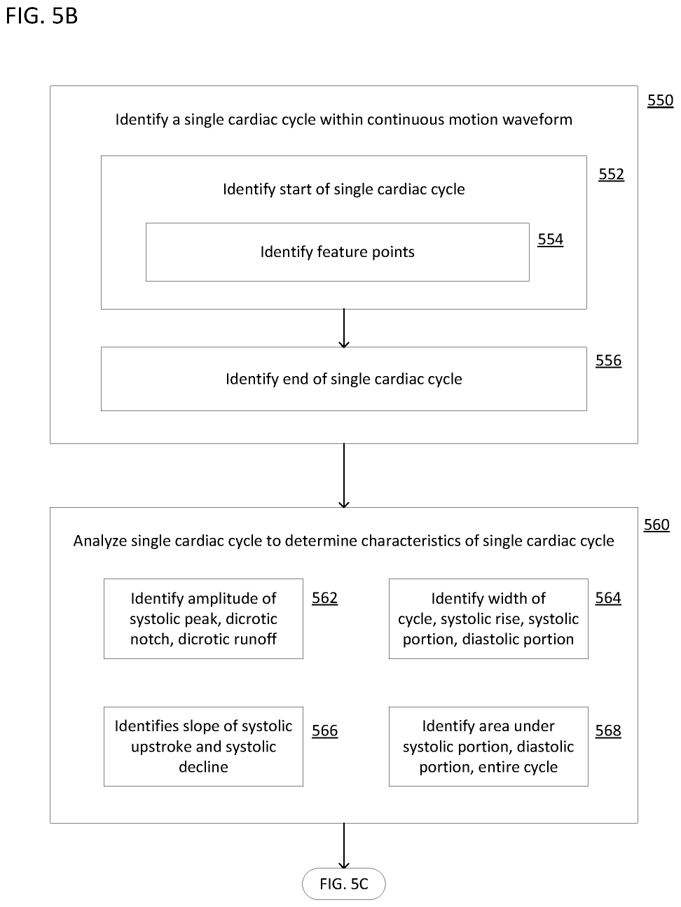

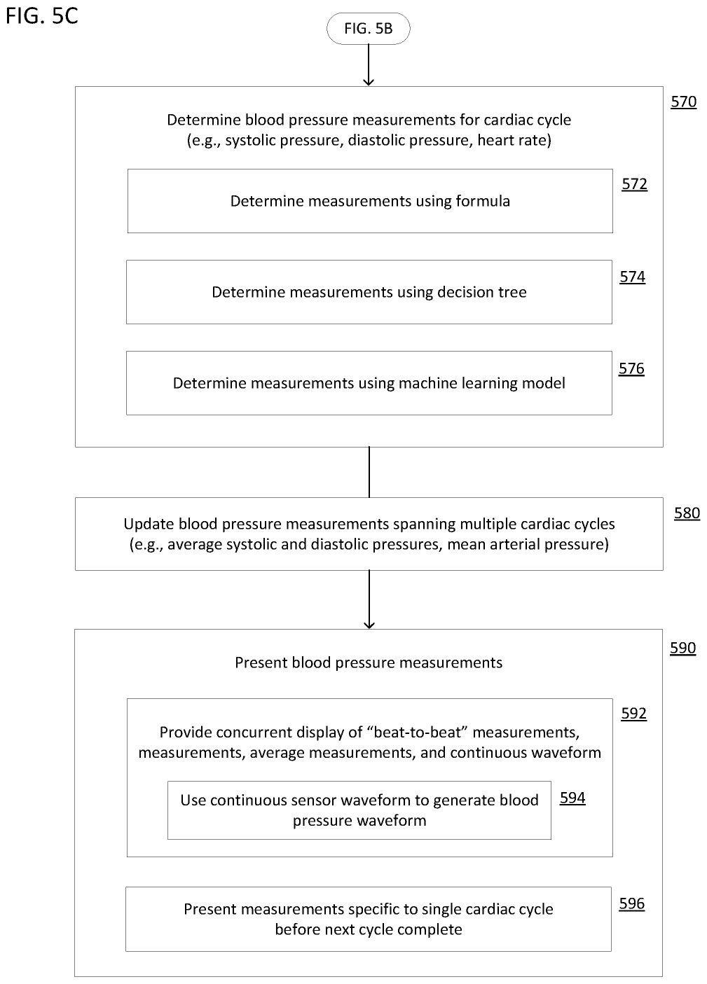

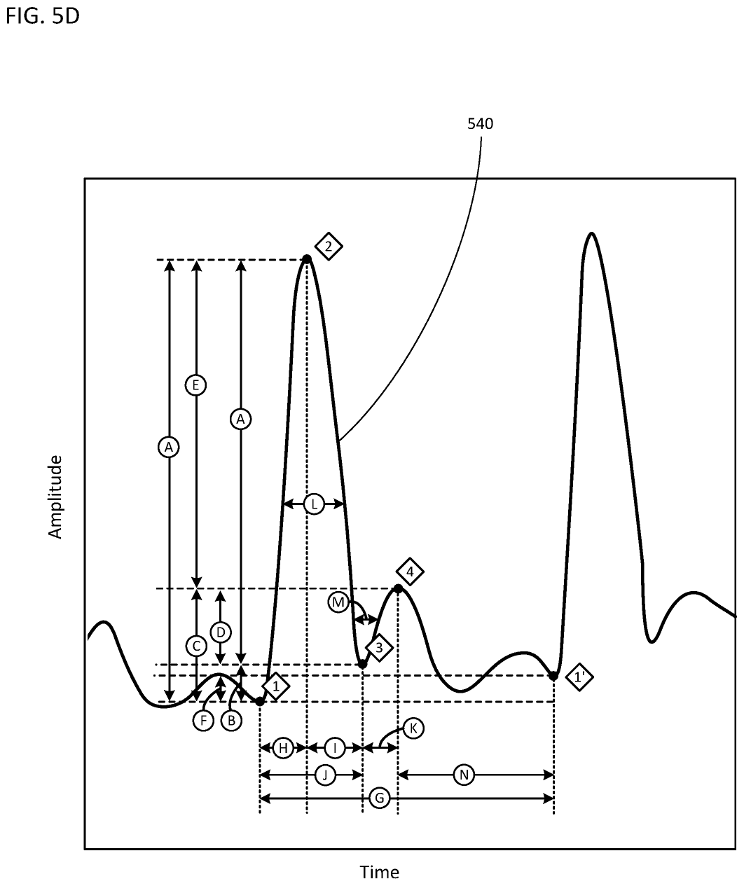

The display 138 may further provide a specific indication of an amount of hold-down force that the monitoring device 102 is currently applying against the skin surface 114. Such a measure may prompt a user to adjust the device to have a desired hold-down pressure within a pre-defined range. In the FIG. 1 example, such a hold-down force indication may be provided as a “Force” field 144 on display 138, which displays a calculated amount of hold-down force that is being applied by the monitoring device 102 against the skin in arbitrary units. The display 138 in FIG. 1 shows a hold-down force value (“Force”) of 694, again in arbitrary units. The hold-down force may be calculated by the monitoring device 102, for example, from an output of the micro-motion sensor 120, and may be communicated from the monitoring device 102 to the local device 104 for presentation on the display 138. Details of an example of how the monitoring device 102 may calculate the hold-down force value 144 is described below in connection with FIGS. 4 and 5.

The remote or back-end system 106 may be used for remote monitoring of the subject for which the monitoring device 102 is applied as well as concurrently other subjects being monitored, and/or for storage of personal heath data from multiple subjects, for example, as a medical health record including blood pressure information and other biometric data collected by the monitoring device 102. The remote system 106 may receive measured blood pressure and other biometric information from the monitoring device 112 (for example, using cellular communications such as may be used under an Internet-of-Things (“IoT”) protocol), or via the local device 104 (for example, where the local device is utilizing a local protocol such as Bluetooth® communications with the monitoring device 102). The remote system 106 may be accessible by the subject (e.g., a patient) and/or the patient's health care provider. In addition, blood pressure and other biometric information for many subjects may be accessed for example in anonymous form for other research and healthcare service purposes. As such, the remote or back-end system 106 may include communication modules to control and execute communications to and from the local device 104 and/or the monitoring device 102. The remote or back-end system 106 may also include a control and processing module to perform control and processing functions required of the system 106, a user interface including visual displays for remote real-time monitoring, and user data storage to store the previously mentioned user files and any aggregated health data files.

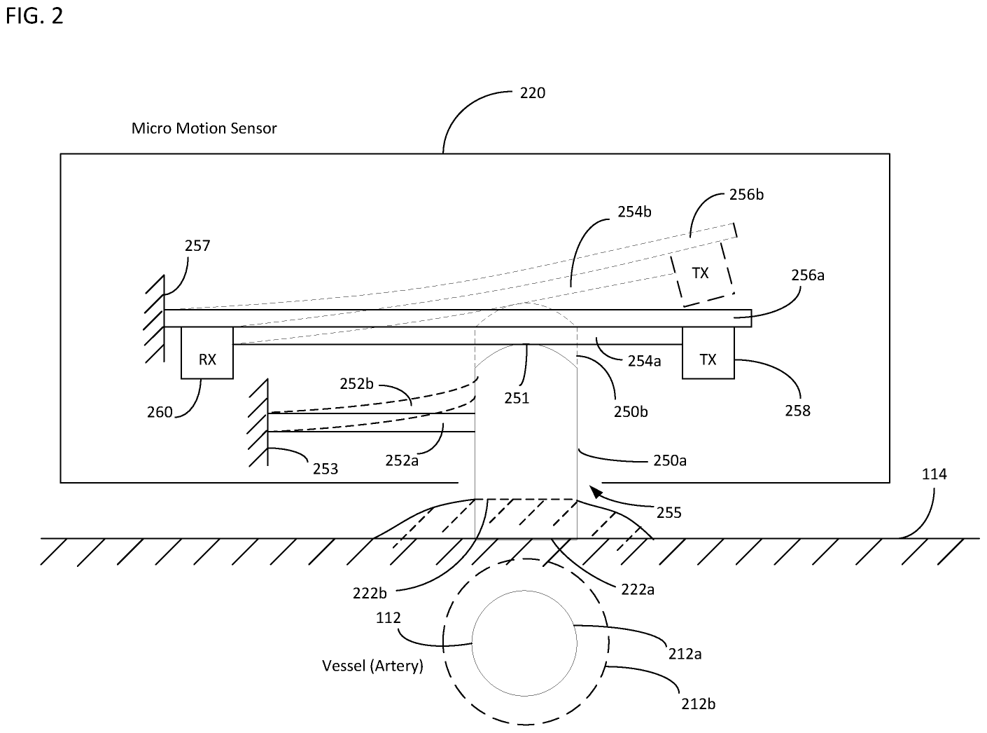

In FIG. 2, there is shown a simplified diagram of one example of a micro-motion sensor 220 that may serve as, for example, the micro-motion sensor 120 provided in the monitoring device 102 shown in FIG. 1. In this example, the micro-motion sensor 220 is an opto-electronic sensor that employs a technology known as optical power modulation (“OPM”) in which the forces of skin surface motion are applied against the side of an optical waveguide so as to modulate the optical power output of the waveguide. U.S. Pat. Nos. 7,822,299 and 8,343,063 to Borgos et al., having their assignee in common with the present patent application, describe examples of OPM and other opto-electronic micro-motion sensor systems and methods, and are incorporated by reference into the present application.

Referring to FIG. 2, the micro-motion sensor 220 includes a button or pad structure 250 (shown in two positions, a resting position 250a and a deflected position 250b) which rests against the skin surface 114 adjacent the underlying artery 112, and a leaf spring structure 252 (also shown in two positions, a resting position 252a and a deflected position 252b). Not shown in FIG. 2 is other housing structure of the device which may rest against the skin surface 114 at a location that is peripheral of where the button or pad structure 250 contacts the skin surface 114.

In the embodiment of FIG. 2, a skin-contacting lower surface 222 (shown in two positions, 222a and 222b) of the button or pad structure 250 is shaped to be generally flat. In other implementations, the lower skin-contacting surface 222 may be rounded and additionally or alternatively may be smaller or larger in cross-section than what is depicted in the figures herein. The leaf spring structure 252 has one end connected to, or in effect carrying, the button 250. The opposite end of the leaf spring structure 252 is connected or otherwise held stationary with respect to housing structure 253, which may be accomplished by connecting the leaf spring structure 252 directly or indirectly to the housing structure of the micro-motion sensor 220 itself, or, referring to FIG. 1, connecting the leaf spring structure 252 indirectly or directly to the housing structure of the sensing module 121 or of the monitoring device 102.

The button or pad structure 250 is configured so that it may be deflected, or in other words, is movable back and forth (up and down in relation to FIG. 2). An outer portion of the button 250 extends external of the sensor 220 through an opening 255 in the sensor's housing structure. The button 250, in use, has a skin contacting surface 222 on an outer portion of the button 250 (shown in two positions, a first position 222a and a second position 222b) which is applied against the surface of the subject's skin 114 adjacent the underlying artery 112. As illustrated in FIG. 2 in exaggerated form for illustration purposes, the artery 112 expands from a rest state 212a to an expanded state 212b as a pressure pulse propagates through the artery. The forces caused by the artery expansion may be measured by micro-motion changes on the skin surface, again illustrated in FIG. 2 in exaggerated form. The button 250 in FIG. 2, again, is shown in two positions, namely, a first or “resting” position 250a shown in solid lines (corresponding to when a pulse is not propagating through the artery 112), and a second or “deflected” position 250b shown in dashed lines (corresponding to when a pulse is propagating through the artery 112).

Blood pressure pulses propagating through the artery 112 cause a force at the surface of the subject's skin to be applied against the button 250, as illustrated in FIG. 2 in exaggerated form for illustration purpose. These forces cause the button 250 to be moved (upward in relation to FIG. 2) from the first, resting position 250a. Specifically, the button 250 moves inward, or in other words into the micro-motion sensor 220 housing, toward a second, deflected button position 250b. When the button 250 is deflected as shown for example in position 250b, the leaf spring structure 252 is also deflected as shown in dashed lines of leaf spring structure 252b. Owing to the leaf spring structure 252 being held stationary with respect to housing structure 253, the leaf spring structure 252 causes the button 250 to return toward the first button position 250a as the force caused by a pressure pulse upon the button 250 becomes reduced.

The sensor 220 also includes a flexible optical waveguide 254 (shown in two positions, a first, resting position 254a and a second, deflected position 254b), as well as an optical source or transmitter (Tx) 258 on one end of the waveguide 254 and an optical detector or receiver (Rx) 260 on the opposite end of the waveguide 254. The optical source 258 may be for example a light emitting diode or some other optical source that injects light energy into the optical waveguide 254 to be received by the optical detector 260. The amount of light energy provided by the optical source 258 may be held constant, with the flexing and/or compression of the waveguide 254 modulating the power of optical energy detected at optical detector 260. The flexible waveguide 254 shown in FIG. 2 is provided upon a flexible substrate structure 256 (also shown in two positions, 256a and 256b). One end of the flexible substrate structure 256 is connected or otherwise held stationary with respect to housing structure 257, which may be accomplished by connecting the end of the substrate structure 256 directly or indirectly to the housing structure of the micro-motion sensor 220 itself, or, referring to FIG. 1, connecting the end of the substrate structure 256 indirectly or directly to the housing structure of the sensing module 121 or of the monitoring device 102.

The sensor's button 250 is positioned vis-à-vis the waveguide 254 such that an internal surface 251 of the button 250 bears against a side of the waveguide 254 along the waveguide's longitudinal extent. As such, a force caused by a pressure pulse in the vessel 112 (which causes a force to be applied against the skin contacting surface 222 of the button 250) causes the button 250 to move upward so that the internal surface 251 of the button 250 applies a force against the side of waveguide 254. This force against the side of the waveguide 254 causes the waveguide 254, as well as the substrate structure 256 upon which the waveguide 254 is positioned, to be flexed from the first, resting position 254a/256a toward the second, deflected position 254b/256b. Owing to one end of the flexible substrate structure 256 being held stationary with the housing structure 257, the flexible substrate structure 256 operates effectively like a leaf spring to cause the return of the substrate structure 256 and hence the waveguide 254 toward the first, resting waveguide position 254a and the first, resting substrate structure position 256a, as force caused by the button 250 against the side of the waveguide 254 becomes reduced (because the force caused by a pressure pulse upon the button 250 becomes reduced).

The flexing of the optical waveguide 254—as caused by the force of the internal button surface 251 against the side of the waveguide 254—alters the power of light that exits the waveguide 254. For example, as the waveguide 254 is flexed with the power of the optical source 258 held constant, the optical power received by the optical receiver 260 may be reduced. Optionally or alternatively, the optical waveguide 254 may be manufactured so that it is compressible under application of force on the side of the waveguide 254 and so that it returns to an original uncompressed state after application of the force on the side of the waveguide 254 is removed. As such, the application of force by the internal button surface 251 on the side of the waveguide 254 may cause the waveguide 254 to become compressed (instead of or in addition to flexing), and thereby change (e.g., reduce) the optical power out of the waveguide 254 as detected by the optical detector 260.

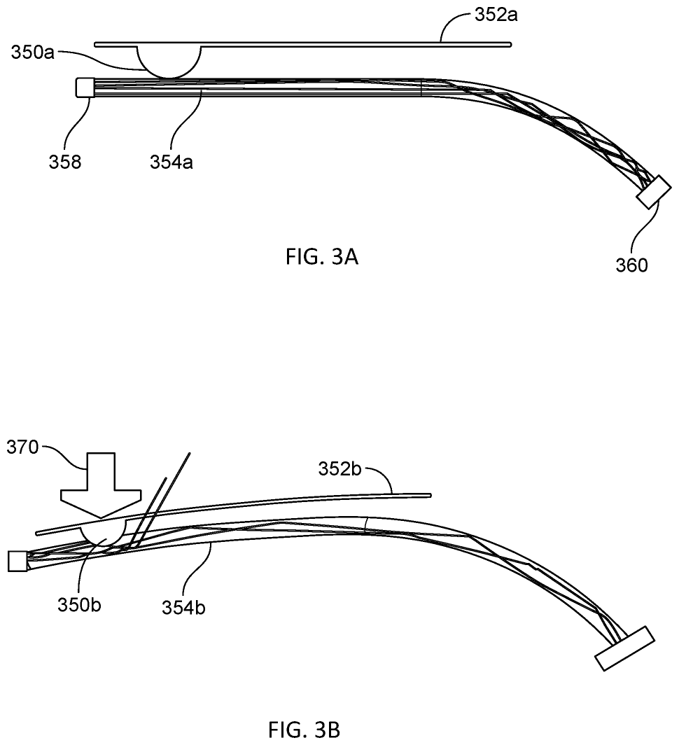

The concept of optical power modulation (“OPM”) by the flexing and/or compression of a waveguide in the presence of a force on the side of the waveguide may be illustrated with reference to FIGS. 3A and 3B. FIG. 3A illustrates an optical waveguide 354a with no deflection. In this state, a leaf spring 352a and button 350a are in a state of rest, as is a flexible portion of the optical waveguide 354a (the flexible portion being that portion which is on the left side of the waveguide 354 in FIGS. 3A and 3B). In FIG. 3B, a force has been applied to the button 350 as indicated by arrow 370, thus moving the button 350 into a deflected state 350b, which causes the flexible portion on the left side of the optical waveguide 354 to be also deflected toward a deflected state 354b.

An optical waveguide 354 such as that shown in FIGS. 3A and 3B in some implementations includes a cladding layer and a core surrounded by the cladding layer, with the cladding layer and core materials comprising different indices of refraction. Optical energy travels within the core, and, as long as the angle of light propagating in the core upon incidence with the cladding is less than a critical angle defined in part based upon the core and cladding layer indices of refraction, light traveling within the core is reflected internally within the core and is not lost by escaping into the cladding layer. This is a concept known in optical waveguide physics as “total internal reflection.” But, if the angle at which light propagating within the core meets the cladding layer exceeds the critical angle, optical energy propagating in the core is lost into the cladding layer, and therefore the optical power received at optical detector 360 is reduced. As can be seen in FIG. 3B in comparison to FIG. 3A, the deflection of the optical waveguide 354b as well as the compression of the optical waveguide 354b causes an optical signal propagating within the core to exceed the critical angle in some locations and instances when the optical signal meets the cladding layer, and thus at least some additional amount of the optical signal is lost into the cladding layer. As such, motion at the skin surface corresponding to a blood pressure pulse in an underlying vessel can be detected with fine precision through the use of OPM micro-motion sensing techniques.

FIG. 4 is a block diagram illustrating an implementation of a body worn or applied monitoring device 402, which may for example serve as the monitoring device 102 in the FIG. 1 implementation. The monitoring device 402 includes a housing 415 encompassing substantially the entirety of the components contained therein. One side 408 of the housing 415 is defined herein as a bottom or underside 408, which is the side of the monitoring device 402 that is applied against a surface of a person's skin adjacent an artery 112, as shown in FIG. 1.

A skin surface motion sensor 420 is located adjacent the bottom or underside 408 of the monitoring device 402. The motion sensor includes an optical source 458 that produces optical energy such as light directed toward an optical waveguide 454 such as an optical fiber. The waveguide 454 transmits optical energy received from source 458 to an optical detector 460. The detector 460 senses received optical signal and produces an output signal indicative of the magnitude of optical power in the optical signal received. The output signal may, for example, be analog or a series of sampled digital values indicative of the received optical signal.

A button or pad structure 450 is positioned on the underside 408 of the monitoring device 402 and bears against the side of the optical waveguide 454 to alter and modulate the optical power of the optical signal received by the optical detector 460. A modulating force indicative to the pulsing movement of the surface of the skin acts upon a skin contacting surface 422 of the button or pad structure 450, which determines the amount of force that the button or pad structure 450 applies against the side of the optical waveguide 454. In some embodiments, the micro-motion sensor 420 may utilize optical power modulation (“OPM”) techniques discussed above, wherein the force applied by button or pad structure 450 on the side of the optical waveguide modulates the optical power output from the waveguide 454 and received by detector 460. In other embodiments, the micro-motion sensor 420 may utilize optical speckle techniques, wherein a speckle image from the optical waveguide 454 is projected to the detector 460, and the speckle produced is altered depending upon the amount of force being applied by the button or pad structure 450 against the side of the optical waveguide 454.

The micro-motion sensor 420 in the example implementation of FIG. 4 is included within a micro-motion sensing module 421, which also includes various sensor signal processing components for example a microprocessor unit (MPU) 462 (which may be part of a control and processing module 472) that continuously receives the output signal provided by the sensor 420 and continuously generates data 464 comprising a digitized sensor waveform and various blood pressure measurements on a beat-to-beat basis (e.g., systolic pressure, diastolic pressure, heart rate, and mean arterial pressure), as described previously. As such, the micro-motion sensing module 421 is configured to enable its integration into various monitoring systems that may make use of the sensor module 421.

The MPU 462 may include analog front-end circuitry 466 which may perform filtering of the optical detector analog output signal, perform analog-to-digital conversion of the analog output signal, and perform other processing functions to produce a digitized waveform. The MPU 462 also includes a mathematical processing component 468 which may continuously perform mathematical processing functions upon the digitized waveform generated by the front-end circuitry 466, and generate a continuous digitized blood pressure waveform (arteriogram) and/or various “beat-to-beat” blood pressure measurements—for example, systolic blood pressure, diastolic blood pressure, mean arterial pressure, pulse pressure (which may be the diastolic pressure subtracted from the systolic pressure), heart rate, etc.—for each cardiac cycle represented in the digitized sensor waveform.

Generally, the mathematical processing component 468 may, in some embodiments, analyze a shape of a portion of the continuous motion waveform that corresponds to a single cardiac cycle of one heartbeat to obtain measurements for predefined shape parameters (where the shape parameters specify characteristics of the shape of the portion of the continuous motion waveform that corresponds to the single cardiac cycle of one heartbeat), and calculate a blood pressure measurement for the single cardiac cycle of the heartbeat based on the obtained measurements for the predefined shape parameters from the portion of the continuous motion waveform that corresponds to the single cardiac cycle of one heartbeat.

Data 464 characterizing the continuous, beat-to-beat blood pressure of the subject may then be stored via data transfer over a local data bus 470 in memory or data storage 474, which may comprise, for example, buffer memory utilized in the process of displaying a “real time” representation of the filtered and digitized sensor signal (or a continuous blood pressure signal generated therefrom) on the device 402 itself or utilized in the transmission of data to an external device such as the local device 104 or remote system 106 shown in FIG. 1, and/or data storage memory where the data may be stored in the device 402 for later download and/or display.

The monitoring device 402 may include a power source 428 as described in connection with the power source 128 of FIG. 1. The monitoring device 402 may also include an additional control and processing module or unit 472 (of which the MPU 462 functions discussed above may be a part) that may, for example, control operation of the device 402 or work in combination with MPU 462 in generating blood pressure measurement information. The monitoring device 402 may also include one or more communication modules 430, as described in connection with FIG. 1 to communicate with, for example, a local device 104 or remote backend system 106 as shown in FIG. 1. The monitoring device may also include other body parameter sensing devices. Specifically, the monitoring device 402 may include one or more position sensor(s) 476, utilizing for example gyroscope or accelerometer devices, so as to determine the position of the monitoring device 402 (for example, is the device 402 positioned at the level, or in other words, elevation, of the subject's heart). In a case where the monitoring device 402 determines, using information from the position sensor 476, that the monitoring device 402 is not at the level of the subject's heart, the monitoring device 402 or an external device may use that position sensor 476 data to transform blood pressure measurements taken at the sensed position so that those values may be transformed using mathematical calculations into what the blood pressure measurement values would have been had the monitoring device 402 been at the level of the subject's heart.

The monitoring device 402 may also include an activity sensor 478, which may be useful to determine the activity level of the subject when blood pressure measurements are being taken. For example, using the activity sensor 478, optionally along with heart rate information from the micro-motion sensor 420, the device 402 may assess whether the patient is relaxed and has been still for a sufficient period of time, such that blood pressure measurements taken by the monitoring device 402 may be of diagnostic value. In addition, information about activity level from the activity sensor 478 may be used to compare blood pressure measurements when the subject is in an active state for comparison to other blood pressure measurements taken from other subjects or standards for blood pressure at that activity level.

The monitoring device 402 may also include a temperature sensor 480 to sense the temperature of the subject. Temperature may provide an indication of stress or activity level, and similarly may be used to determine if the subject is in a state where a blood pressure measurement with diagnostic value may be taken, or may be used to transform or compare blood pressure measurements taken at various temperature levels. The monitoring device 402 may also include an electrocardiogram (ECG) sensing system. A monitoring device 402 that combines continuous and accurate blood pressure information in combination with continuously monitoring ECG information may be able to provide useful diagnostic and predictive information about the subject. In addition, the monitoring device 402 may further contain algorithms to evaluate both continuous blood pressure information and continuous ECG information for the subject and provide, if a dangerous condition is sensed such as atrial fibrillation, an alarm to the subject via an alarm 431 on the monitoring device 402, or alternatively or additionally, the monitoring device may transmit information about the alarm condition to a remote device such as a local device 104 or a remote back-end system 106 for remote monitoring of patients as shown in FIG. 1. Various other sensors 484 may also be included in the monitoring system 402.

FIGS. 5A-C show a flowchart illustrating methods by which a monitoring device such as the device 102 of FIG. 1 or the device 402 of FIG. 4 may be operated. It will be appreciated that the operations shown in the FIGS. 5A-C flowchart may be performed in an order other than that shown in FIGS. 5A-C, and not all operations need be present in every implementation.

Referring to FIG. 5A, at 502 the method begins with the monitoring device, such as the device 102 of FIG. 1 or the device 402 of FIG. 4, being put in place, so that it is worn by the subject in the case of a wearable device or is applied against the patient in the case of a body applied device that is not worn by a subject but rather may be pressed against the subject by an operator such as a healthcare provider. By way of example referring to FIG. 1, the outer surface 122 of the micro-motion sensor 120 may be applied against the skin 114 of a subject adjacent an artery 112. Or referring to FIG. 4, an outer surface 422 of a button or pad structure 450 as shown in FIG. 4 may similarly be placed against the skin of a subject adjacent an underlying artery.

At 504 the monitoring device 102, 402 may be paired with another device, such as the local device 104 shown in FIG. 1 or a remote backend system 106 shown in FIG. 1. This pairing may be a Bluetooth® pairing process, for example in the case of the local device 106 near enough to the monitoring device 102, 402 to enable Bluetooth® communications, or alternatively the pairing may be a process over a WiFi or cellular network, wherein a monitoring station as part of a remote back-end system 106 may be configured to monitor the subject wearing the monitoring device 102, 402. At this point, with pairing having been accomplished, the monitoring device 102 may communicate to the local device 104 or the remote system 106, and/or vice versa.

At 506 the monitoring device 102, 402 may be activated or “woken up” to start a monitoring process. For example, the monitoring device 102, 402 may be activated by a user activating a button or other interface on the device 102, 402 to start the monitoring process. Alternatively, the monitoring device 102, 402 may be activated by an external device such as a local device 104 or a remote system 106 sending a communication to the monitoring device 102 to “wake” the monitoring system up, for example, from a sleep state that preserves battery power.

At 508 the monitoring device 102, 402 may assess the hold-down force and positioning of the monitoring device 102, 402 against the skin. By way of background, in some implementations, the monitoring device 102, 402 continuously monitors blood pressure on a beat-to-beat basis with a constant hold-down force being applied by the device 102, 402 against the surface of the skin adjacent an artery (e.g., with the hold-down force being in a range of 5-15 mm Hg or other suitable hold-down forces, as described in additional detail below). In a case of a wrist worn device, for example, the device may be placed on the wrist with the strap positioned to apply a desired hold-down force within the desired range.

In some embodiments, the analog output signal from the micro-motion detector (e.g., 260 in FIG. 2 or 460 in FIG. 4) may be analyzed to determine a current amount of hold down force being applied. For example, a baseline level of the analog output may be used to identify the amount of hold-down force that is being applied by the device 102, 402 against the surface of the skin. The monitoring device 102, 402 may identify the baseline level of hold-down force from the analog output as a lowest value output by the sensor 120 over a period of time (e.g., over a single cardiac cycle, over a pre-determined number of cardiac cycles, or over a predetermined number of seconds, such as 3 seconds), and may determine whether that baseline level of force falls within an acceptable range.

It should be understood that the hold-down force may be applied by a component attached to a spring (e.g., button 250 attached to spring 252 in FIG. 2) and that the force applied to the surface of the skin may vary slightly due to the spring tension changing as the surface of the skin displaces the component (and therefore the spring) due to blood pulsing through the underlying artery. As such, what is meant by a constant hold-down force is an application of a hold-down force that is constant at a given skin displacement over a period of time. For example, the device 102, 402 would apply the same hold-down force each time the skin and button 250 reach a “resting” position over the course of multiple cardiac cycles (and similarly would apply the same hold-down force each time the skin and button 250 are at a “fully displaced” position over the course of the multiple cardiac cycles). This is in contrast with cuff-based measurement systems that steadily increase the pressure in the cuff over multiple cardiac cycles using actuator, and then decrease the pressure in the cuff over multiple cardiac cycles. At least some of the embodiments described in this disclosure do not activate an active actuator to modify the amount of force that is applied for a given level of displacement during a measurement period (e.g., multiple cardiac cycles).

A display may be provided on the device 102, 402 or on an application program of a local device 104 that may provide an indication to the user of the amount of hold-down force currently being applied, and whether the amount of hold-down force will provide a valid blood pressure measurement. In the example of FIG. 1, the “Force” measurement 144 is such an indication, with the units in this example being arbitrary. In this case, there may be a range of numbers between a minimum and a maximum hold-down force within which the “Force” number 144 must reside in order for the device 102, 402 to indicate that it will work properly.

Regarding the minimal amount of hold-down force, operation of the device 102, 402 may require a modest amount of hold down force to ensure that the relevant portion of device 102, 402 (e.g., surface 122, 422) remains in contact with the subject's skin and/or that the button 250, 450 maintains a minimal, baseline amount of deflection of the waveguide 254, 454 rather than, for example, the relevant portion of device 102, 402 occasionally bouncing out of contact with the subject's skin. As discussed below, devices may be designed to acquire blood pressure measurements from different locations/arteries on a subject, and in embodiments that are designed for acquiring blood pressure measurements from arteries that are deeper or shallower in the body than the radial (wrist) artery, greater minimal-hold down pressures may be required. For device 102, 402, which can acquire measurements from the radial artery, the minimal amount of hold-down force may be 0.1, 0.3, 0.5, 0.8, 1, 2, 3, 4, 5, 6, or 7 mm Hg, for example. If the “Force” number 144 is determined to be indicative of a hold-down force condition that is below the minimal amount of hold-down force, the strap or a wrist-worn device for example should be tightened.

The maximum amount of hold-down force depends on device construction and the type of blood vessel over which the device 102, 402 is applied. Operation of the device 102, 402 is premised upon the device not changing the shape of the underlying artery and/or unduly constricting the underlying artery. This is in contrast to tonometry, where a much greater force is applied against the artery (e.g., 60 mm Hg or more) and that force is intended to be great enough to flatten (i.e., partially occlude) the artery. The amount of hold-down force that would unsatisfactorily change the shape of or constrict the artery, however, depends on how deep the artery is within the body. For example, the carotid artery (neck) and the renal artery (back) are deeper than the radial artery and therefore permit greater hold-down forces than a force applied over the radial artery. The temporal artery is shallower than the radial artery and therefore permits a lower maximum hold-down force than that allowed for the radial artery. For a radial artery, the maximum amount of hold-down force applied by device 102, 402 may be 8, 10, 13, 15, 18, or 20 mm Hg. Any of these maximum hold-down forces may be combined with any of the above-described minimum hold-down forces, to generate various different acceptable hold-down force ranges. In some embodiments, the device 102, 402 determines whether the hold-down force falls under a maximum value and does not determine whether a minimal hold down force is satisfied (e.g., whether the hold-down force is less than 20 mm Hg).

If the “Force” number 144 is indicating that the hold-down force is above the allowable range, the strap of the wrist-worn device, for example, should be loosened. As described above, too high of a hold-down force, and the device 102, 402 may constrict the underlying blood vessel 112, which can affect the accuracy of the blood pressure reading. Accordingly, at 508 if it is determined that the hold-down force is incorrect, the device 102, 402 may be adjusted at 510. The device 102, 402 may be adjusted manually, for example, by adjusting a strap or triggering a structure that step-wise either increases or decreases the hold-down force by a set and precise amount. Alternatively, the device 102, 402 may include automatic adjustment mechanisms to either increase or decrease the hold-down force using, for example, motor-controlled adjustment mechanisms. The automatic adjustment mechanism may change the hold-down force using the motor-controlled adjustment mechanisms without receiving user input while the motor-controlled adjustment mechanisms perform operations to change the hold-down force and settle upon an acceptable hold-down force (although user input may initiate the automated adjustment process).

Regarding positioning of the skin contacting portion 122, 422 of the device 102, 402 vis-à-vis the underlying artery, the device 102, 402 may again analyze the nature of the analog output signal from the optical detector (e.g., 260 in FIG. 2 or 460 in FIG. 4) to determine if the device 102, 402 is positioned correctly vis-à-vis an underlying artery (e.g., whether the device 102, 402 is centered over the underlying artery or is improperly located to the side). Analysis of the analog output of the optical detector 260, 460 may show that the motion signal is not significant or distinct enough to accurately measure blood pressure. For example, the device 102, 402 may determine that the analog output signal provides a correct baseline level—indicating that the hold-down force is appropriate—but that the peak-to-peak amplitude may not satisfy a pre-determined, minimal threshold, which can be indicative of the device 102, 402 being positioned to the side of the artery.

If the device 102, 402 determines for example that the positioning is inadequate, the device may provide an indication showing that the positioning is inadequate on the device 102, 402, or alternatively, the device 102, 402 may send a signal to an external device such as the local device 104 of FIG. 1, and the local device may provide an indication of whether the position is correct or not, for example, by use of a “Placement” indicator being shown in a green color or a red color. In the case of the position being determined to be incorrect (again, at 508 of the flowchart of FIG. 5), the device may be adjusted at 510. In various implementations, the adjustment at 501 may be accomplished manually by a user, or automatically by the device 102, 402 itself. As for the latter (automatic adjustment), the device 102, 402 in some implementations may include multiple micro-motion sensing devices (120 in FIG. 1; 420 in FIG. 4), and the adjustment at 510 may comprise selecting a different one or combination of the micro-motion sensing devices. Alternatively, the device 102, 402 may include motorized structures for automatic adjustment of the device to optimize the micro-motion sensor's positioning.

If the hold-down force and positioning of the device 102, 402 is correct, it may then be determined at 512 if conditions are suitable for blood pressure monitoring. In a case of blood pressure being taken under current medical standards, it is desirable for example that the subject has rested for 3-5 minutes, that the subject be sitting with both feet on the ground or laying down, and various other conditions (not talking, not smoking, etc.). In some implementations, the device 102, 402 or an application on a local device (e.g., smartphone) 104 may query the user about conditions, and require user responses that conditions are suitable. Alternatively or additionally, various sensors 476, 478, 480, 482, 484 (FIG. 4) may be utilized to determine if conditions are suitable. If conditions are not suitable, at 514 the device 102, 402 may institute a waiting period and thereafter the conditions may be checked again at 512.

If the conditions are suitable as determined at 512, at 516 the monitoring of the subject's blood pressure may commence for a pre-defined period of time or indefinitely. The pre-defined period of time may be 30 seconds, for example. During the course of a day, it may be prescribed or desirable to take readings for 30 seconds every 20 minutes or a half an hour, for example. During the time that the blood pressure measurement is being taken, blood pressure measurement information may be stored in local memory (for example, data storage 474 in FIG. 4) and/or the information may be streamed to the local device 104 for display or storage there. In the case of the monitoring being for an indefinite period of time, the monitoring may continue until the user stops it, or until for example the device 102, 402 determines monitoring need not continue. In some cases, the device 102, 402 may determine that conditions of the subject are such that continuous monitoring must continue, because for example a patient whose blood pressure is being monitored may be in danger of entering a hypotensive state in surgery.

Next at 518 the micro-motion sensor's analog output may be processed to generate a digital continuous motion waveform. The micro-motion sensor's analog output identifies an amount of light that transmitted all the way through the waveguide 454 over time. As such, this signal provides an indication of the amount of force that the button 450 is applying against the side of the optical waveguide 454 over time, which generally is representative of the motion of the surface of the skin adjacent an artery. The processing of the analog signal may include analog and/or digital signal filtering, for example, to remove noise or to remove effects that may be attributed to motion of the subject rather than attributed to motion caused by pulsing in the underlying artery (e.g., as determined in comparison to motion identified using other motion sensors provided with the device 102, 402). The processing at 518 may further include analog-to-digital conversion and other processing to generate a digitized motion waveform from the analog signal. For example, the device 102, 402 may invert the continuous motion waveform so that a blood pressure peak is represented by a peak rather than a trough in the continuous motion waveform (e.g., because positive displacement of the skin and sensor represents a blood pressure peak, but that positive displacement would cause a reduction in the amount of light transmitting all the way through the waveguide). The processing at 518 may be performed, for example, by analog front-end circuitry 466 in FIG. 4 and perhaps also the mathematical processing component 468.

At 520 the filtered and digitized sensor waveform may be processed on a beat-to-beat basis. This may be performed, for example, using a mathematical processing component 468 of an MPU 462 (FIG. 4). The processing may first select a portion of the digitized motion waveform corresponding to a single cardiac cycle, and process just that one cycle of the digitized motion waveform without the need for calibration to calculate various blood pressure measurements and other biometric information for that cardiac cycle. In other words, the processing may not require that a separate blood pressure measurement be taken by other means, such as a cuff-based blood pressure measurement system, in order for the blood pressure measurements for each cardiac cycle to be determined. The pressure measurements and other biometric information for that cycle may include, for example, systolic pressure, diastolic pressure, pulse pressure, mean arterial pressure, and cardiac output. The processing of each cardiac cycle may be performed as the digitized motion waveform is being generated, or in other words, in real time, such that a display of the blood pressure and other biometric information along with the digitized pressure waveform may be provided immediately and on a beat-to-beat basis as the subject is under monitoring.

The processing at 520 to determine blood pressure and other biometric information on a beat-by-beat basis, without calibration, may employ an evaluation of various pre-defined shape parameters for a cycle of a digitized motion waveform. Because the digitized motion waveform may correspond to motion of the subject's skin surface and the underlying artery, the shape of the digitized motion waveform may approximate the shape of a waveform that would indicate blood pressure within the underlying artery. (Although the correspondence may not be exact, for example, because subjects with high blood pressure may have rigid arteries which may limit displacement of the subject's skin as a blood passes through a blood vessel, in contrast to subjects with less-rigid arteries and presumably lower blood pressure and displacement.) Accordingly, at least some of the features of the digitized motion waveform may correspond to features present in a waveform that identified actual blood pressure, and the features of the digitized motion waveform may therefore be analyzed using terminology typically specific to analysis of blood pressure waveforms even though the motion waveform identifies motion and not directly blood pressure.

Some shape parameters in the digital motion waveform that are analyzed may include, by way of example, (1) rise-time or slope information for the waveform as the digitized motion waveform rises to the systolic peak; (2) the width of the systolic pulse at a specified height of the systolic pulse (e.g., mid-point or some other point) in comparison to the overall period of the cycle; (3) the fall-time or slope information for the digitized motion waveform as the motion waveform falls from the systolic peak; and (4) the shape and/or amount of dip in a dicrotic notch, which is a small downward deflection in an arterial pulse immediately before a secondary upstroke corresponding to a transient increase in aortic pressure upon closure of the aortic valve, as shown in the waveform in the display 138 of FIG. 1. Various other shape parameters may also be utilized.

The predefined shape parameters to be utilized in the processing at 520 and an algorithm that provides a coefficient or weighting value to each of the predefined shape parameters may be determined in a clinical study in which motion waveforms are taken from a range of patients with known blood pressure measurement values and may be aided by machine learning techniques. This may include supervised machine learning processes. Refinement of the predefined shape parameters and an algorithm to apply to the shape parameter measures may occur over time as further subjects with known pressure measurements are obtained. As such, it is possible to provide a continuous blood pressure monitoring system that provides blood pressure measures on a beat-to-beat basis, or in other words, for each cardiac cycle, without the need for calibration of the blood pressure monitoring system for a particular patient. For example, the blood pressure monitoring system and methods disclosed herein do not require that a separate blood pressure measurement be taken by another system in order to calibrate the system for a particular subject.

At 522 the digitized motion waveform represented by the digitized signal waveform and beat-to-beat blood pressure measures for each cardiac cycle may then be continuously stored in memory (e.g., in data storage 474 in the device 402) and/or displayed in real time. The real-time display may be provided on the device 102, 402 itself. The display may be generated by generating the digitized signal waveform for display. Additionally or alternatively, at 520, the device 102, 402 may generate a representation of a blood pressure waveform from the digitized signal waveform, for example, by scaling the motion waveform for representation on a graph-type visual display with blood pressure values on a vertical axis and time on a horizontal axis. Further waveform transformation functions utilizing some or all of the shape parameters discussed above may be created and applied to transform a continuous sensor output waveform into an accurate continuous blood pressure waveform (arteriogram). Additionally or alternatively, numerical values for the blood pressure measures for each cardiac cycle may be displayed in continually updating form for each cardiac cycle, along with average measures over a number of cardiac cycles (for example, the last 10 cardiac cycles).

At 524 the device 102, 402 may continuously or periodically transmit the sensor waveform, the BP waveform, and/or beat-to-beat blood pressure measurement information to a local device 104 or a remote device 106 such as a cloud-based system for storing medical records and/or managing patient care. As such, the data transferred to these devices may be stored and/or displayed in real-time or later on a display device in connection with those external systems, such as on the display 138 of the remote device 104.

While the above discussion of items 520, 522, and 524 provides a high-level overview of the “beat-by-beat” analysis, FIGS. 5B-C show a flowchart that provides additional detail regarding the beat-to-beat analysis by device 102, 402, with discussion of the FIGS. 5B-C flowchart following with reference to items 550 through 596.

At 550, the device 102, 402 identifies a single cardiac cycle within the continuous movement waveform. Although the continuous motion waveform represents an intensity of light measured by sensor 260 over time, the intensity of light corresponds to skin movement caused by the subject's heart beating. As such, a portion of the continuous motion waveform that represents a single cardiac cycle may be identified. An example mechanism to identify a single cardiac cycle is to identify a start of a single cardiac cycle within the continuous motion waveform (item 552) and identify an end to the same single cardiac cycle within the continuous motion waveform (item 556).

Identifying the start of a single cardiac cycle can involve analyzing the continuous motion waveform to identify one or more pre-determined feature points. FIG. 5D illustrates an example continuous motion waveform 540, and example feature points within that waveform 540 are the start of the systolic upstroke (Feature #1), the systolic peak (Feature #2), the dicrotic notch (Feature #3), and the peak following the dicrotic notch (Feature #4). Any one or more of these features (or others) may be identified using various techniques for mathematically analyzing the continuous motion waveform 540, for example, by identifying local minimums and/or local maximums. For example, the system may identify the systolic peak (Feature #2) as a local maximum over a sliding window equal to the length of a cardiac cycle, a portion thereof (e.g., 60% of a cardiac cycle), or longer than a typical cardiac cycle (e.g., 500% thereof, which would involve identifying multiple local maximums representing multiple respective systolic peaks). From identification of the systolic peak (Feature #2), the system may traverse the continuous motion waveform 540 back in time to identify a local minimum that represents the beginning of the systolic upstroke (Feature #1). Conversely, the system may traverse the continuous motion waveform 550 forward in time from the systolic peak (Feature #2) to identify a subsequent local minimum that represents the dicrotic notch (Feature #3). The peak following the dicrotic notch (Feature #4) may be a local maximum that follows the dicrotic notch (Feature #4).

Other suitable processes may be performed to identify these or other features points within the continuous motion waveform, and the system may not necessarily identify all feature points at this stage in processing. A single such feature point, however, is flagged as the start to the cardiac cycle (item 552), and a subsequent identification of the same feature point in a subsequent cardiac cycle is flagged as the end of the cardiac cycle (item 556) and therefore the start of the next cardiac cycle. In the example illustrated in FIG. 5D, Feature #1 indicates the start of the cardiac cycle and the next occurrence of that same feature (i.e., Feature #1′) indicates the end of the cardiac cycle and the start of the next cardiac cycle. The portion of the continuous motion waveform that corresponds to a single cardiac cycle is referred to as a wavelet.

At 560, the device 102, 402 analyzes the wavelet to determine characteristics of the wavelet. Determining these characteristics may involve identification and use of the above-discussed feature points (e.g., the Features #1 through #4 that are illustrated in FIG. 5D), or feature points determined therefrom (e.g., a feature point 30% of the way between Feature #1 and Feature #2). The system may determine these feature points at any suitable time (e.g., during the identification of the wavelet cycle as discussed at 550, as a batch process prior to analyzing the wavelet to determine its characteristics, or piecemeal as each characteristic of the waveform is determined). The characteristics can represent a variety of wavelet measures (e.g., shape measurements), such as amplitude of the wavelet at certain locations, width of the wavelet at certain locations, slope of portions of the wavelet, etc., as discussed in additional detail below.

At 562, the device 102, 402 identifies the amplitude of various portions of the wavelet. As a few examples, and with reference to the wavelet 540 illustrated in FIG. 5D, the device may identify amplitude values for the following characteristics:
Characteristic A: Amplitude between the beginning of the systolic upstroke (Feature #1) and the systolic peak (Feature #2).
Characteristic B: Amplitude between the beginning of the systolic upstroke (Feature #1) and the dicrotic notch (Feature #3).
Characteristic C: Amplitude between the beginning of the systolic upstroke (Feature #1) and the peak following the dicrotic notch (Feature #4).
Characteristic D: Amplitude between the dicrotic notch (Feature #3) and the peak following the dicrotic notch (Feature #4).
Characteristic E: Amplitude between the systolic peak (Feature #1) and the peak following the dicrotic notch (Feature #4).
Characteristic F: Amplitude between the beginning of the wavelet (Feature #1 in this example) and the end of the wavelet (Feature #1′ in this example).

The amplitudes in these examples are measured between features that represent local maximums and minimums, but characteristics be calculated from features that are located between each of the above-described features. For example, the system may calculate characteristic A as the amplitude between a location 10% up the systolic upstroke to a location 90% up the systolic upstroke (or other symmetric or asymmetric portions of the systolic upstroke or other portions of the wavelet, with the locations selected as a percentage or absolute value offset from a local minimum/maximum along the time/x-axis).

At 564, the device 102, 402 identifies the width of various portions of the wavelet. As a few examples, the device may identify width values for the following characteristics:
Characteristic G: Width of the entire wavelet, in this example between the beginning of the systolic upstroke (Feature #1) and the beginning of the next systolic upstroke (Feature #1′).
Characteristic H: Width of the systolic upstroke, between the beginning of the systolic upstroke (Feature #1) and the systolic peak (Feature #2).
Characteristic I: Width of the systolic decline, between the systolic peak (Feature #2) and the dicrotic notch (Feature #3).
Characteristic J: Width of the systolic peak, between the beginning of the systolic upstroke (Feature #1) and the dicrotic notch (Feature #3).
Characteristic K: Width between the dicrotic notch (Feature #3) and the peak following the dicrotic notch (Feature #4).
Characteristic L: Width of the systolic peak at a certain height (e.g., at 50% of the amplitude of the systolic peak).
Characteristic M: Width of the dicrotic notch at a certain height (e.g., at 50% of the amplitude between Feature #3 and Feature #4).
Characteristic N: Width of the diastolic runoff, from the peak following the dicrotic notch (Feature #4) to the next systolic upstroke (Feature #1′)

The width may be represented as elapsed time or other appropriate values, for example, sampled sensor values or computation cycles. As described above with respect to the amplitudes and illustrated with respect to Characteristic L, the widths may be calculated from features that are not local minimums or local maximums. For example, Characteristic L is calculated as the width between features that are located at 50% of the amplitude of the systolic peak (e.g., 50% of the amplitude between Features #1 and #2). As other example, the above-described widths could be calculated as the width between 5% and 95% of the amplitude separating any two reference feature points (or other symmetric or asymmetric proportions of an amplitude separated any two reference points, with the locations selected as a percentage or absolute value offset from a local minimum/maximum along the amplitude/y-axis).

At 566, the device 102, 402 identifies the slope of various portions of the wavelet. As a few examples, the device may identify slope values for the following characteristics:
Characteristic O (not shown): Slope of the systolic upstroke, between the beginning of the systolic upstroke (Feature #1) and the systolic peak (Feature #2) (e.g., Characteristic A/Characteristic H).
Characteristic P (not shown): Slope of the systolic decline, between the systolic peak (Feature #2) and the dicrotic notch (Feature #3) (e.g., (Characteristic A−Characteristic B)/Characteristic I).
Characteristic Q (not shown): Slope between the dicrotic notch (Feature #3) and the peak following the dicrotic notch (Feature #4) (e.g., Characteristic D/Characteristic K).
Characteristic R (not shown): Slope of the diastolic runoff, between the peak following the dicrotic notch (Feature #4) and the beginning of the next systolic upstroke (Feature #1′) (e.g., (Characteristic C−Characteristic F)/Characteristic N).

As described above with respect to the amplitudes and widths, the slopes need not be calculated from Features #1 through #4. Rather, the slopes may be calculated from features that are not local minimums or local maximums, and rather may be calculated from feature points that are themselves calculated based on the positions of local minimums or maximums. For example, the slope of the systolic upstroke (Characteristic O) may be calculated between 20% and 80% of the distance from Feature #1 to Feature #2. Other locations from which slopes are calculated may be selected based on symmetric or asymmetric offsets from other reference points, calculated as percentage-based offsets or absolute offsets.

At 568, the device 102, 402 identifies the area under various portions of the wavelet. As a few examples, the device may identify values for the following characteristics:
Characteristic S (not shown): Area under the entire wavelet, corresponding to the width of Characteristic G.
Characteristic T (not shown): Area under the systolic upstroke, corresponding to the width of Characteristic H.
Characteristic U (not shown): Area under the systolic decline, corresponding to the width of Characteristic I.
Characteristic V (not shown): Area under the entire systolic peak, corresponding to the width of Characteristic J.
Characteristic W (not shown): Area under the diastolic runoff, corresponding to the width of Characteristic N.

The wavelet may not begin and end with the same amplitude, as is the case with Feature #1 and Feature #1′ having different amplitudes. As such, the area may be calculated with a base amplitude and lower bounds to the area calculation being set to (1) a lowest of the wavelet beginning and end points, (2) a highest of the wavelet beginning and end points, (3) an amplitude level half-way between the wavelet beginning and end points, (4) an imaginary, sloped line connecting the wavelet beginning and end points, or (5) the base value generated by the sensor (e.g., such that the wavelet beginning and end points each have positive values when measured with respect to the base sensor value).

At 570, the device 102, 402 determines blood pressure measurements for the wavelet based on values determined for each of the characteristics (the values are sometimes called waveform or shape “measures” or “measurements” in this disclosure). Example blood pressure measurements include systolic blood pressure, diastolic blood pressure, heart rate, and mean arterial pressure. These measurements may be specific to the wavelet, such that the blood pressure measurements are based on characteristics of only the wavelet and do not account for characteristics of any other cardiac cycles. There are various techniques to determine the blood pressure measurements once values have been determined for the above-described characteristics, for example, using a formula (item 572), a decision tree (item 574), or a machine learning model (item 576), which are discussed in turn hereinafter.

At 572, the device 102, 402 determines one or more of the blood pressure measurements by applying values for multiple of the above-described characteristics into an equation that weights the values/characteristics in different manners. An example formula follows, with W denoting a weight value (subscript identifying the respective characteristic), C denoting a characteristic value (subscript identifying the respective characteristic), and X denoting constants: SYSTOLIC=X₁ (W_{A}*C_{A}−W_{K}*C_{K})/(W_{S}*C_{S}−W_{V}*C_{V})+X₂ (W_{P}*C_{P}+W_{Q}*C_{Q}). The systolic and diastolic values may be determined with independent formulas. The specific characteristics to use in a formula and the weight values to apply to the characteristics may be determined through trials in which subjects wear device 102, 402, the device records values for various characteristics, and those values are correlated with beat-to-beat blood pressure measurements recorded at the same time, for example, with a cuff-based system (using for example the auscultation method) and/or an arterial line.

At 574, the device 102, 402 may alternatively determine one or more of the blood pressure measurements by applying values for multiple of the above-described characteristics to a decision tree. An example decision may be whether the amplitude of the systolic peak (Characteristic A) is more than 4 times the depth of the dicrotic notch (Characteristic D). Another example decision may be whether the area under the entire wavelet (Characteristic S) is less than or greater than some predetermined threshold. As such, the decisions may include comparisons of characteristic values to specific thresholds, may include comparisons of characteristic values to each other, or may include a mix of both types of decisions (and potentially other types of decisions). The decision tree may output a numerical value for a particular type of blood pressure measurement (e.g., generate a value of 92 for diastolic pressure), or may output a decision to use one of multiple candidate formulas that are specific to a situation identified by the decision tree (e.g., the formula DIASTOLIC=X₁ (W_{M}*C_{M}−W_{N}*C_{N})+X₂ (W_{P}*C_{P}/W_{Q}*C_{Q}). As described above, the characteristics to use and the weight values to apply to those characteristics may be determined through trials involving subjects and analysis of clinical data.

At 576, the device 102, 402 alternatively or additionally determine one or more of the blood pressure measurements through use of a trained machine learning model. For example, and as described above, trials may be run in which subjects wear the device 102, 402, the device records values for various characteristics, and those values are correlated with blood pressure measurements recorded with a different machine. The recorded characteristic values (with device 102, 402) and blood pressure measurements (with a different machine) can be fed into a machine learning model to train the model. The training can be done using data that has been separated into component cardiac cycles such characteristics for a single cardiac cycle are taken into account in generating a blood pressure measurement for the single cardiac cycle. Alternatively, the training can be done such that a history of characteristics or blood pressure measurements across more than just a single cardiac cycle can be considered when generating a blood pressure measurement for the single cardiac cycle. Once the model has been trained based on information from multiple subjects, the trained model can receive, as an input, values for a same set of characteristics on which the model was trained, and the trained model can output one or more blood pressure measurements. In some examples, one or more trained machine learning models may be combined with a decision tree, and different machine learning models may be selected for different situations or subject criteria (e.g., different trained models used based on whether the subject is still or has been moving, and different trained models used based on whether the subject is male or female).

At 580, the device 102, 402 updates blood pressure measurements that span multiple cardiac cycles. For example, the device may determine an average systolic blood pressure value over a certain window of time or a certain number of cardiac cycles (e.g., 10 cardiac cycles), and may re-determine the average systolic blood pressure value after a value specific to a single cardiac cycle has been determined, so that the average systolic blood pressure value takes into account the systolic value calculated for the most-recent cardiac cycle (and does not account for an oldest value, for example, using a sliding window mechanism). Similar mechanisms may be used to determine the average diastolic pressure and the average heart rate.

At 590, the device 102, 402 presents the blood pressure measurements or provides those measurements for display by another device (e.g., a paired local device 104). This presentation may correspond to the visual display 138 that is illustrated in FIG. 1.

At 592, this visual display concurrently presents (1) a continuous waveform, (2) blood pressure measurements specific to a single cardiac cycle (e.g., SYS=131, DIA=62, HR=75, MAP=85 in FIG. 1), and (3) blood pressure measurements based on information from multiple cardiac cycles (e.g., ASYS=129, ADIA=61, Avg HR=75 in FIG. 1). For example, as the continuous motion waveform slides across the screen, entering or coming into existence at one side and leaving on the other, the blood pressure measurements at the top and bottom of the screen may update at intervals that correspond to completion of processing of a cardiac cycle. In some examples, the visual display presents multiple beat-specific blood pressure measurements for multiple, respective beats. For example, the visual display may concurrently present three systolic measurements that correspond to three cardiac cycles illustrated by a continuous waveform on the screen at that moment. In some examples, the visual display may concurrently present content from two of the three types of information described above (e.g., only SYS=131, DIA=62, and the continuous waveform 140). In some examples, the visual display may present content from only one of the three types of information described above (e.g., only HR=75).

At 594, the device 102, 402 may modify the continuous motion waveform (which may indicate the intensity of light received by sensor/receiver 260 with some processing performed thereon, for example, to remove noise and invert the waveform) to generate a continuous blood pressure waveform that identifies the intensity of blood pressure in the artery 112 at different moments in time. For example, given that the systolic value and the diastolic value may be calculated using the above-described techniques and that these blood pressure values correspond to the locations in the continuous motion waveform of Features #2 and #1 respectively, the system may generate value for the y-axis. Still, the intensity of the light received by the sensor/receiver 260 may not linearly track skin displacement, and skin displacement may not linearly track changes in arterial blood pressure. These non-linearities can occur because the force applied by, for example, the spring 252 in sensor 120, 220 may not have a linear relationship to displacement of the button 250 (and therefore the skin surface). Moreover, the displacement of the skin may not have a linear relationship to an increase in arterial blood pressure. The relationships between these different parameters can be determined through user trials, and non-linear mappings of sensor/receiver intensity to blood pressure can be determined. Accordingly, the device 102, 402 may use the non-linear mappings to perform a non-linear vertical stretching/transformation of the continuous motion waveform so that the y-axis and the values represented thereby illustrate linear data. As an rough illustration, the top half of the waveform may be stretched in the vertical direction while the bottom half of the waveform may be compressed in the vertical direction. The continuous blood pressure waveform may be presented instead of (or in addition to) the continuous motion waveform 140, on either the body worn device 102, the local device 104, the remote system 106, or any combination thereof.

At 596, the visual display may present blood pressure measurements specific to a single cardiac cycle before the next cardiac cycle is complete. In other words, the system may present the “beat-to-beat” measurements in “real-time,” such that the measurements are calculated and displayed for a particular cardiac cycle before the system has recorded an entirety of the next cycle and/or calculated the blood pressure measurements for htat next cycle. Although the system is capable of traversing historically-recorded motion waveforms to identify “beat-to-beat” measurements for that waveform, it is able to identify the blood pressure measurements in real time (both those measurements specific to a single cardiac cycle and those measurements that are based on data that span multiple cardiac cycles).

Returning to the flowchart in FIG. 5A, at 526 the monitored beat-to-beat blood pressure measurement information may be monitored on continuing basis, either alone or in combination with other sensed information, to determine if an alarm condition exists, in which case an alarm may be provided. For example, the monitoring device 102, 402 may continually process the blood pressure information in a patient at risk for stroke to alarm the patient or others to the condition if a stroke may be imminent. Similarly, an alarm may be provided in the event of an atrial fibrillation condition.

At 528 the blood pressure monitoring period that was commenced at 515 ends, because for example a predefined period of time for which the monitoring is to be done has ended, or a user has stopped the monitoring from continuing. At 530 the monitoring device 102, 402 may be deactivated and/or put into a sleep mode.

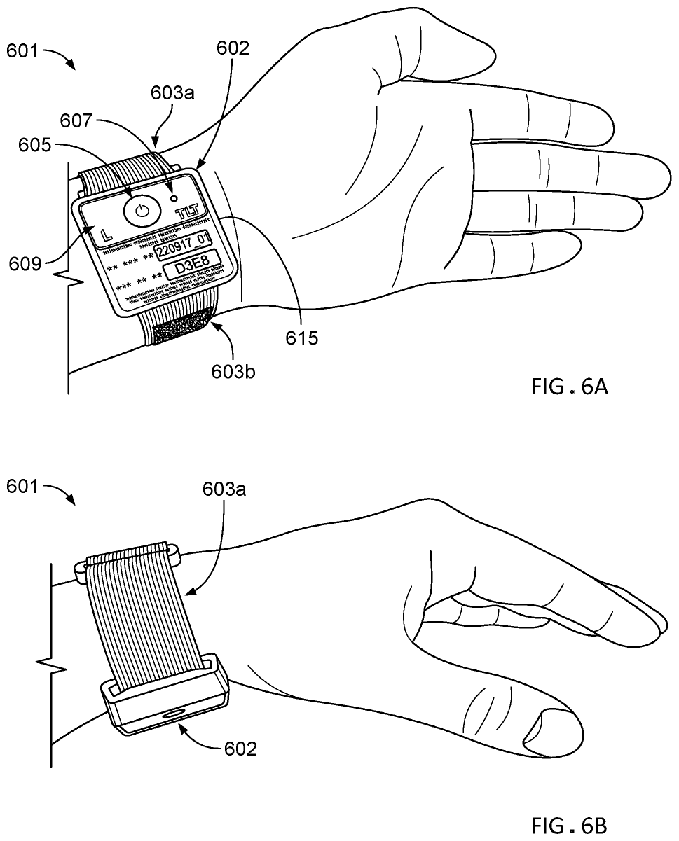

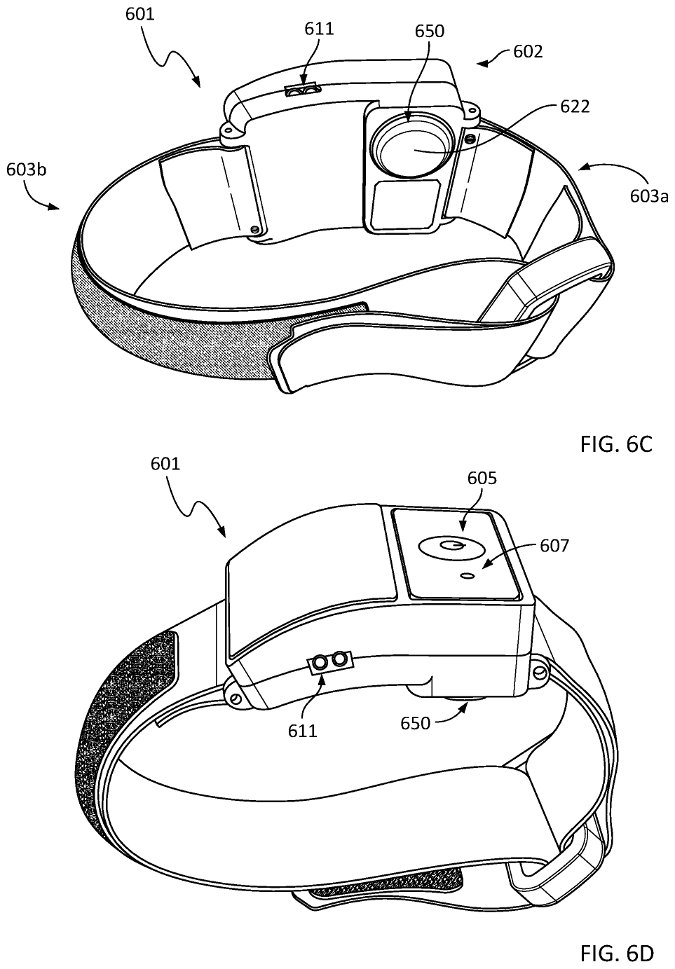

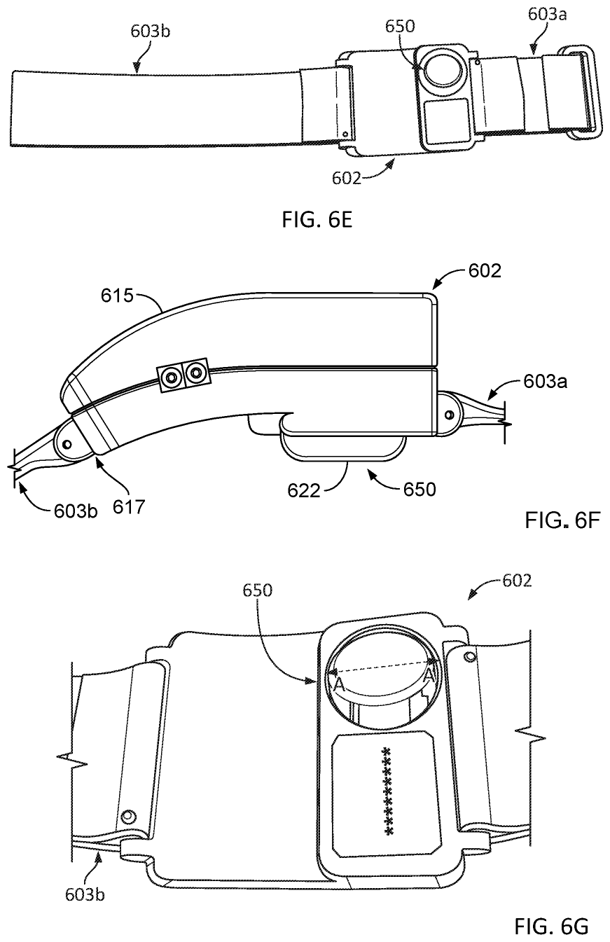

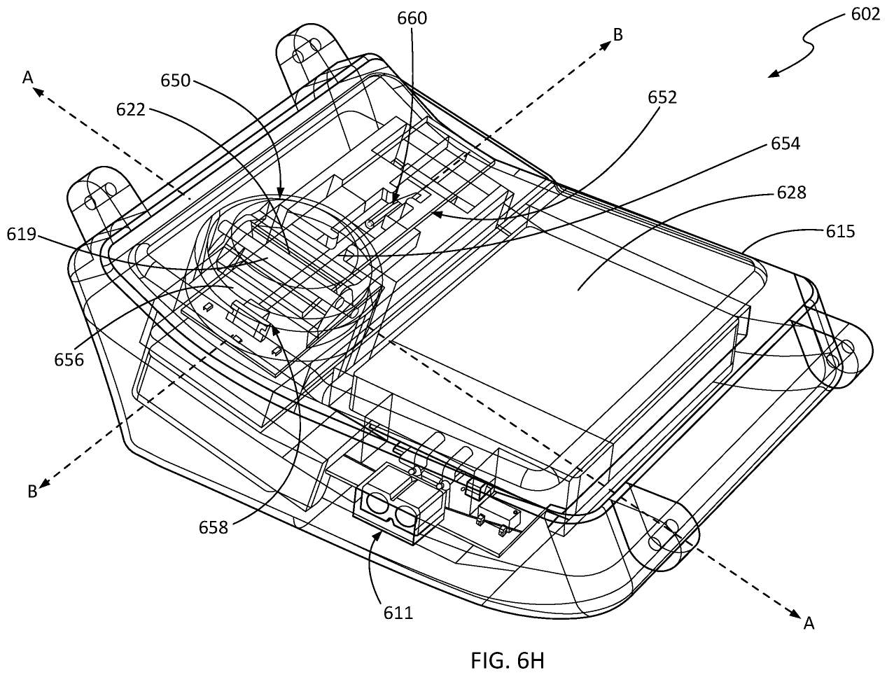

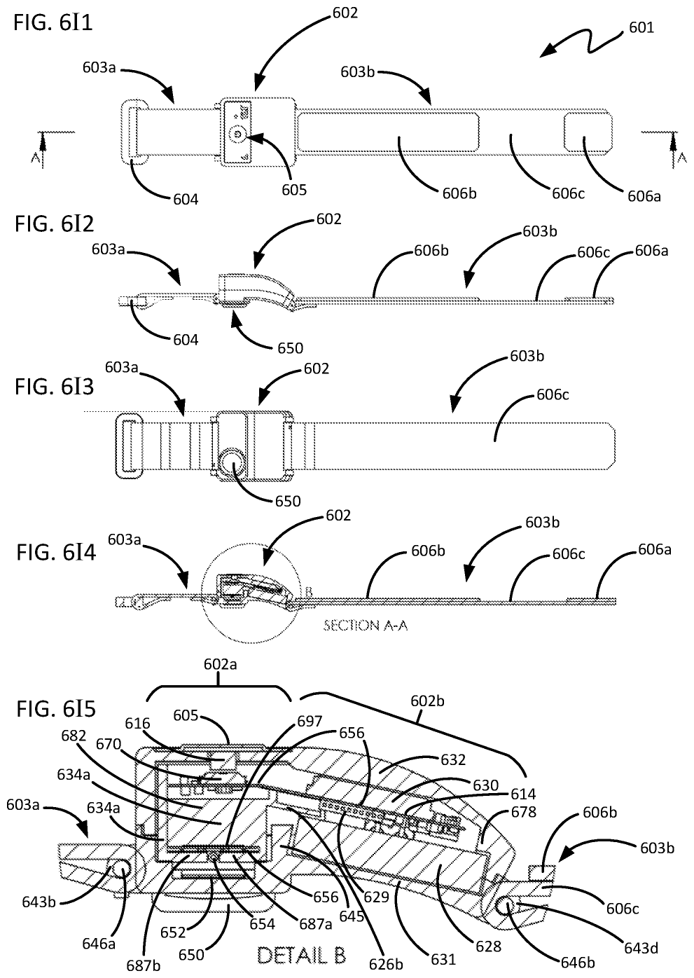

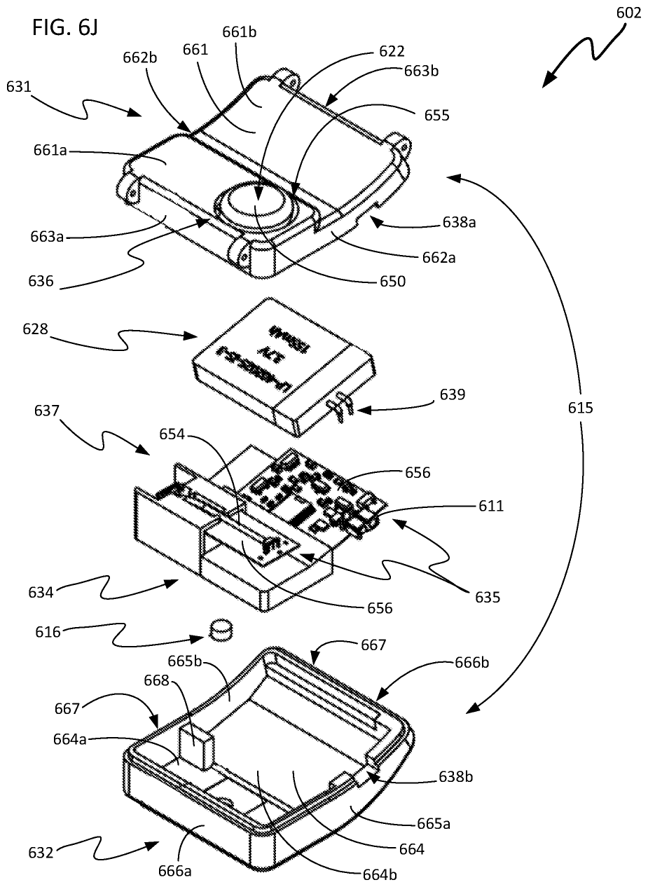

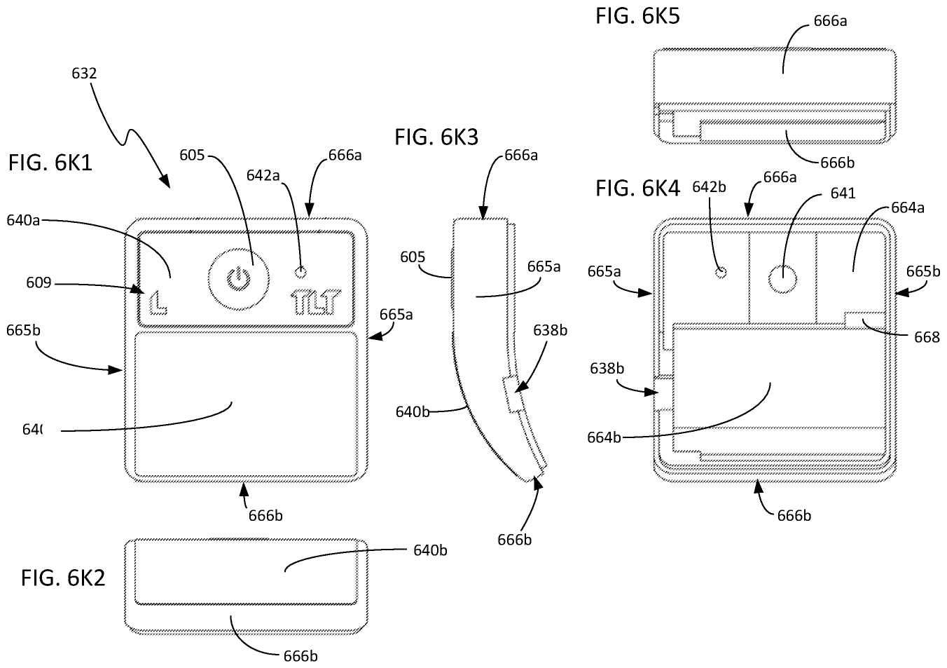

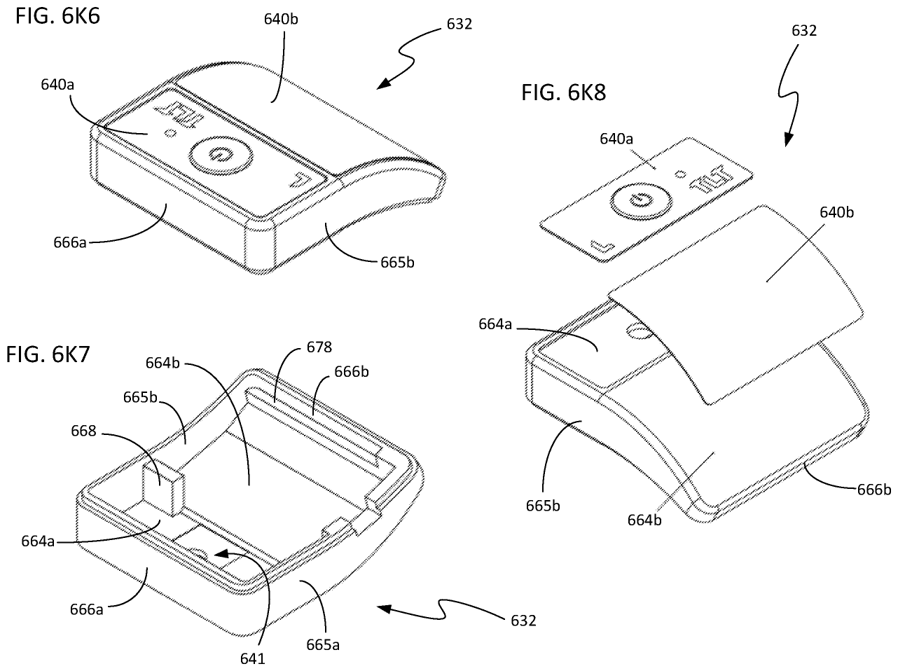

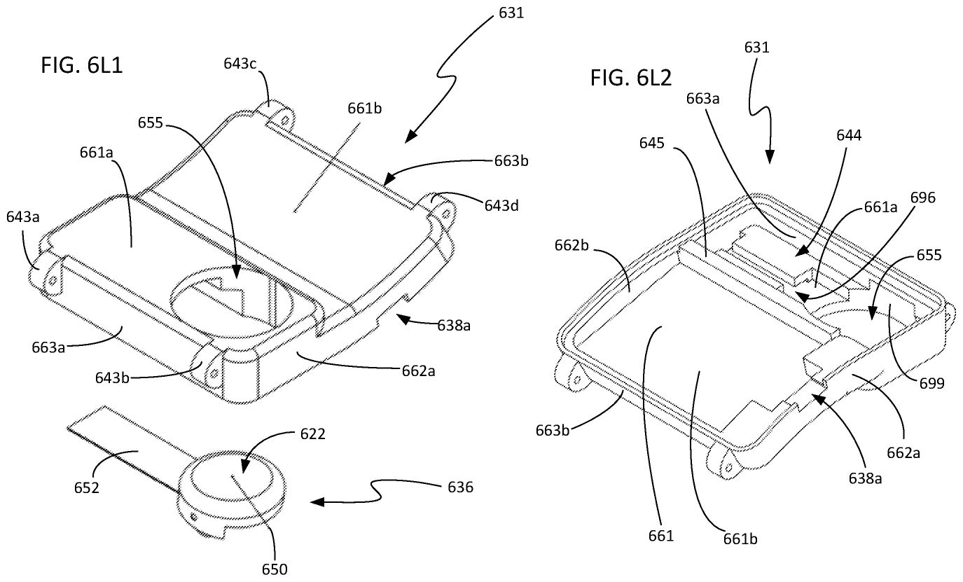

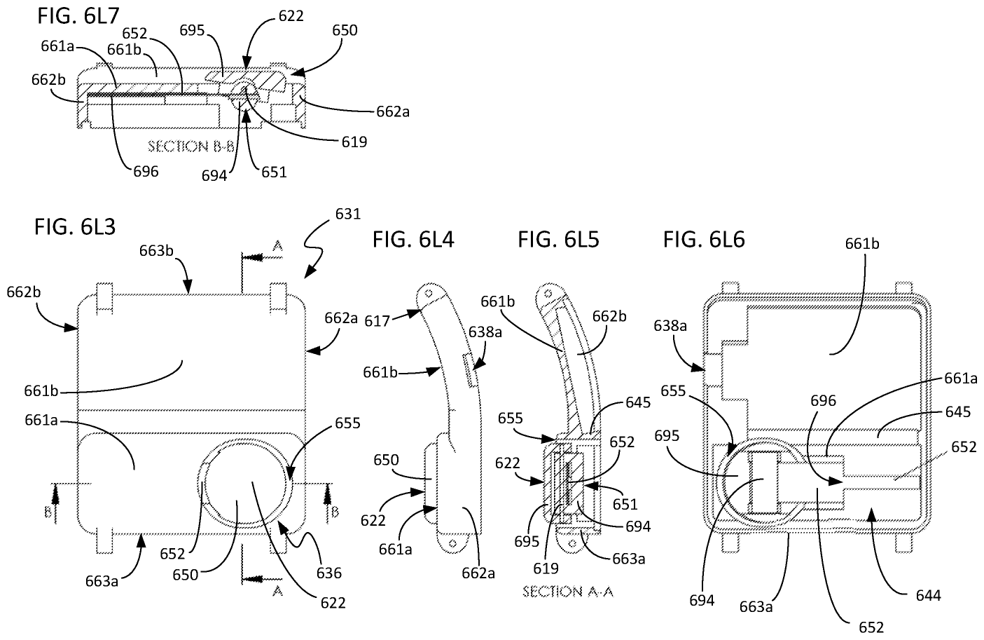

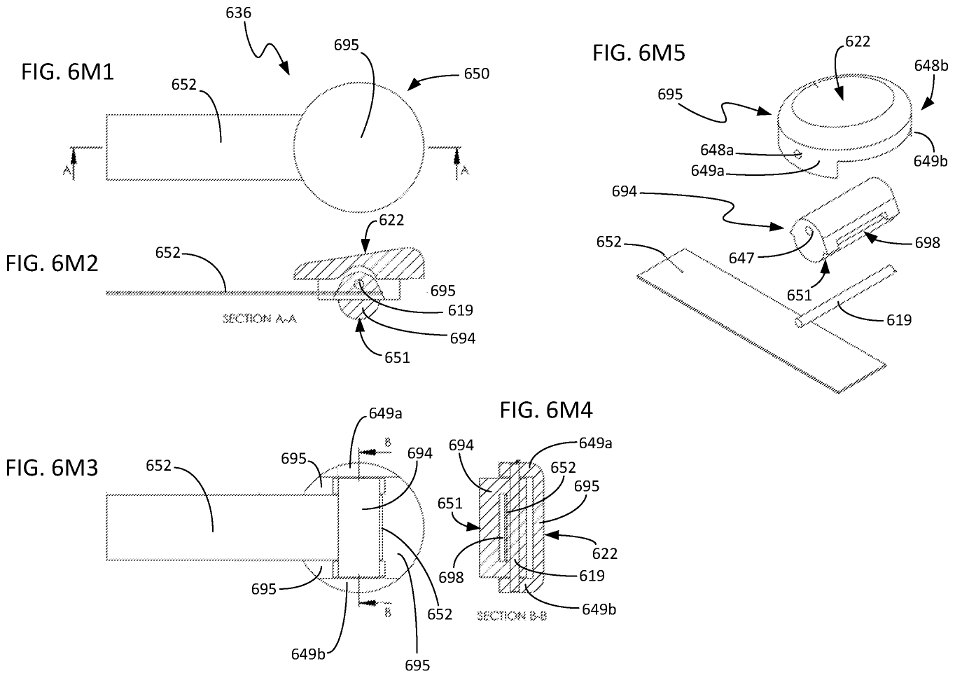

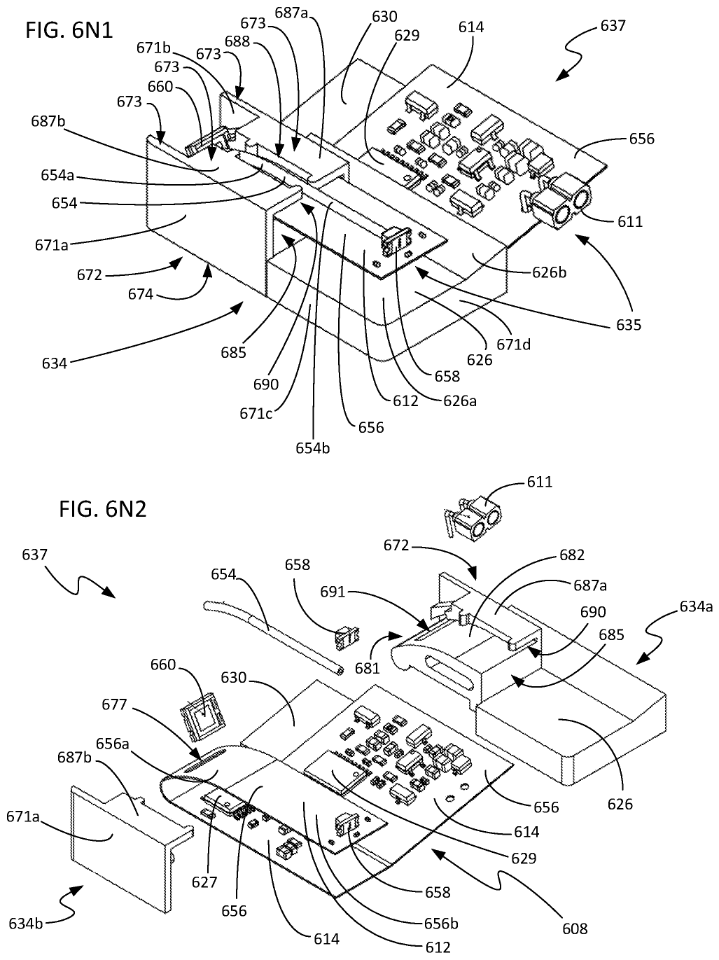

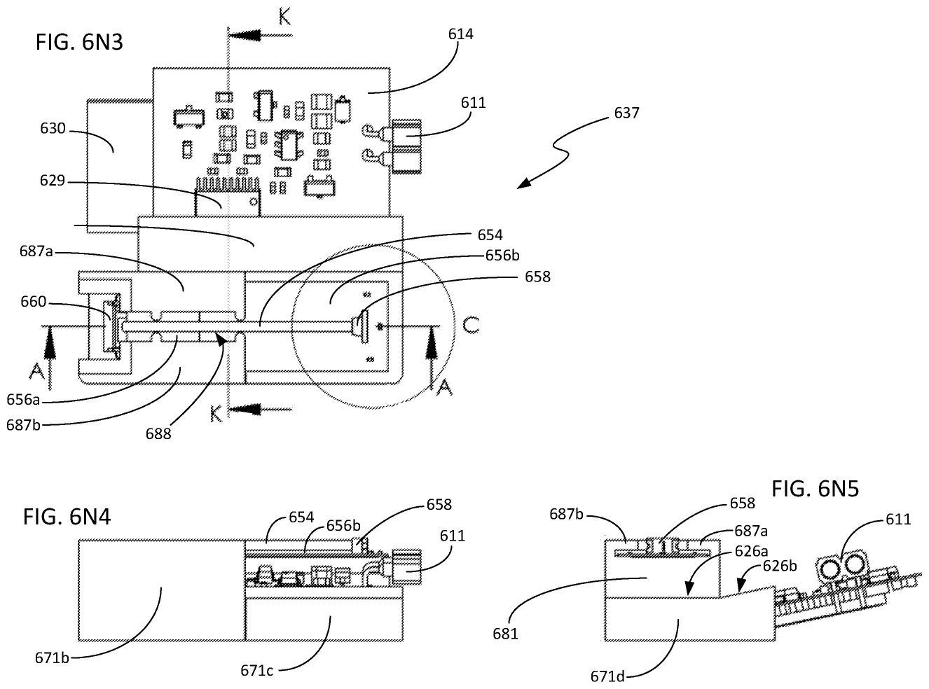

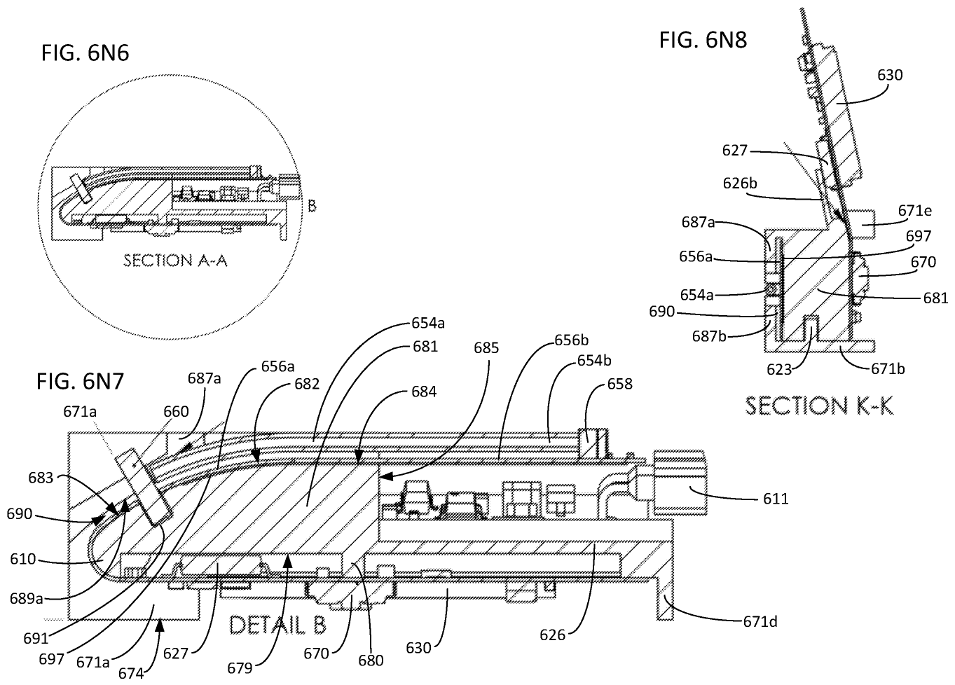

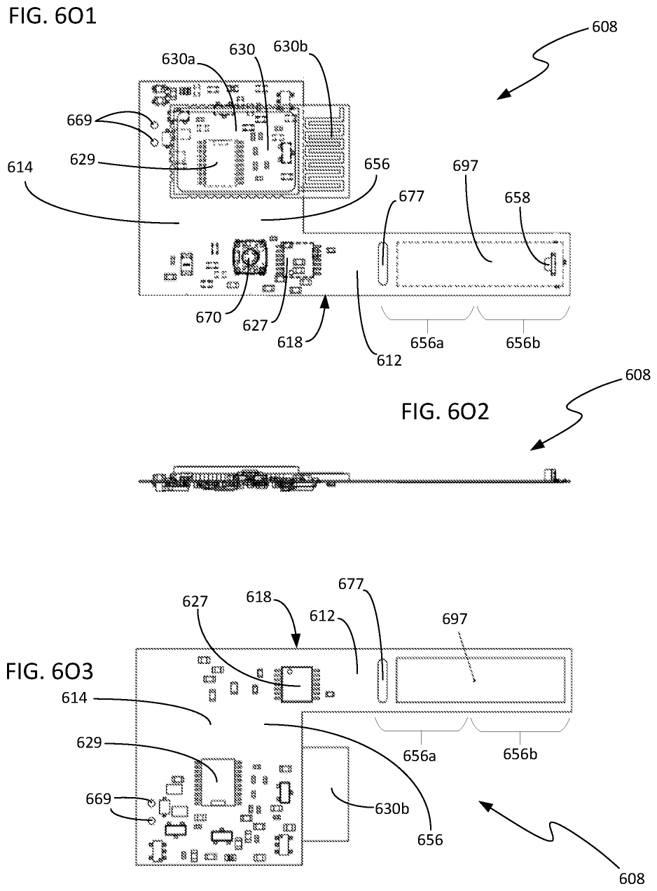

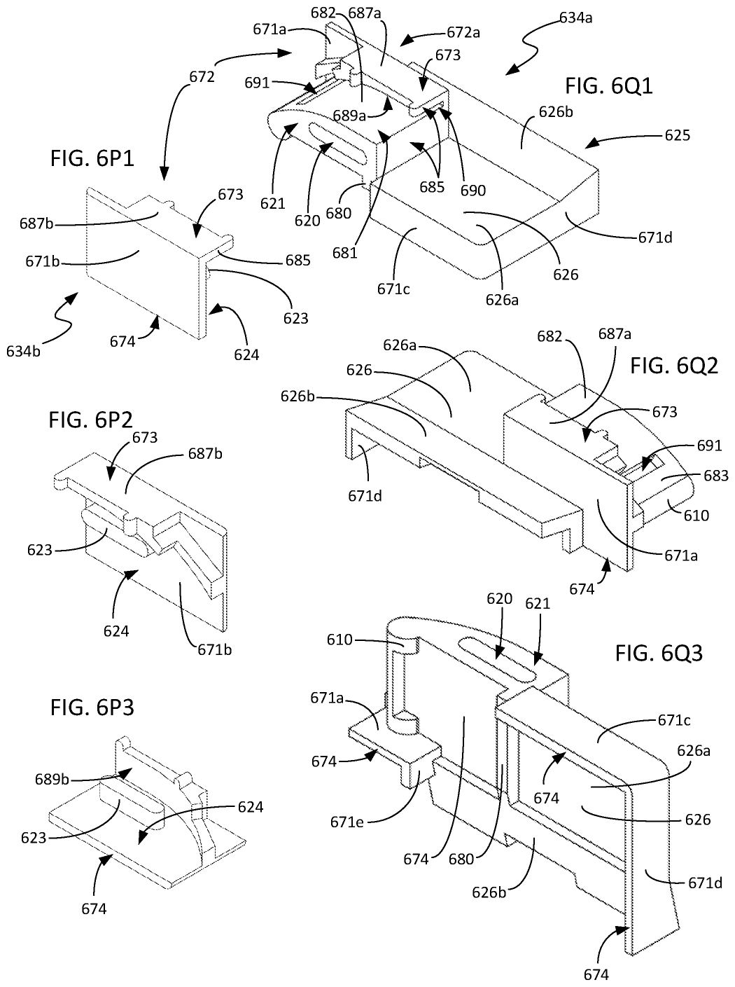

FIGS. 6A-6Q show an example implementation of a wrist-worn system 601 that includes an embodiment of a monitoring device 602 of a type belonging to the monitoring devices 102, 402 shown in FIGS. 1 and 4. The system 601 is designed to be wrist-worn and monitors pressure in the radial artery, which is an artery that traverses the wrist. The system 601 applies the monitoring device 602 against the skin adjacent the radial artery at a location that is on the underside (ventral side) of the wrist. Application of the device 602 is aided by a band 603 comprising first and second straps 603a, 603b that are applied around the wrist. In FIG. 6A, the underside (ventral side) of the subject's wrist and hand is shown, illustrating that the system 601 is worn such that the monitoring device 602 is positioned on the underside (ventral side) of the wrist. FIG. 6B provides a side view of the hand and wrist as well as the system 601, showing the monitoring device 602 again being applied to the underside (ventral side) of the wrist.

As shown in FIG. 6A, the monitoring device 602 includes a housing 615 in which the various components of the device 602 reside. The monitoring device 602 includes a press button 605 situated on an outward facing surface of the device housing 615 that can be pressed by a user to turn the device 602 “on” and “off.” An indicator light 607 also situated on the outward facing surface of the device housing 615 lights up to indicate the device is “on,” and when not lighted indicates the device 602 is “off.” The “L” labeling 609 on the outward facing surface of the device housing 615 as shown in FIG. 6A indicates that the device is designed to be worn on the left wrist. In this implementation, the housing configuration is such that it is intended to be worn on the left hand, given the positioning of a button 650 (see FIG. 6C) that is intended to be positioned against the skin adjacent the radial artery.

FIGS. 6C and 6E show the wrist-worn system 601 in a view showing a skin-facing side of the monitoring device 602, which side has an exposed button or pad structure 650 that is placed in contact with the skin adjacent the radial artery. The button or pad structure 650 has, in this implementation, a generally flat skin-contacting surface 622 on the outer or skin-facing side of the button or pad structure 650. The button or pad structure 650 is similar in concept to the button or pad structure 450 of FIG. 4. The skin-contacting surface 622 is specifically the portion of the device 602 that is placed in contact with the skin adjacent the radial artery. At the side of the housing 615, as shown in FIGS. 6C, 6D and 6F, is a dual-pin port 611 for connecting a charging device, to charge a battery contained within the monitoring device 602 and/or provide power to the device 602.

Referring now to FIG. 6F, there is provided a side view of the monitoring device 602 with portions of straps 603a, 603b to the side, to illustrate the contact points of the monitoring device 602 against the ventral side of a subject's wrist. As mentioned previously, a skin-contacting surface 622 of the button or pad structure 650 contacts the skin surface adjacent the radial artery. The housing 615 also includes a skin-facing bearing surface 617 on a lateral portion of the skin-facing surface of the housing 615 that is opposite of where the button or pad structure 650 is positioned. Generally, there are at least two portions of the monitoring device 602 that will be in contact with the ventral side of the wrist when the system 601 is properly adjusted with the straps 603a, 603b applying a proper amount of hold-down force by the button or pad structure 650, which hold-down force that does not construct the underlying artery (e.g., a range for example of 2-20 or 5-15 mm Hg or some other appropriate range as discussed above). Specifically, the skin contacting portions of the monitoring device 602 that will be in contact with the ventral side of the wrist are at least the skin-contacting surface 622 of the button 650 and the housing skin-facing bearing surface 617.

As illustrated firstly in FIG. 6G but also in more detail in subsequent figures, the button or pad structure 650 is configured so that it is pivotable to allow the skin-contacting surface 622 of the button or pad structure 650 to make better contact with the skin surface adjacent the artery to accommodate a variety of different wrist anatomies of users. The pivoting occurs about an axis labeled A-A in FIG. 6G.

Referring now to FIG. 6H, a perspective diagram of the monitoring device 602 is provided in a way that it is possible to see through some of the components to see the internal components and configuration of the device 602. In particular, FIG. 6H shows that the button or pad structure 650 is connected to a leaf spring 652 (similar to leaf spring 252 in FIGS. 2 and 352 in FIG. 3). The button or pad structure 650 has two parts (illustrated in more detail in later figures), with a pin structure 619 connecting the two button parts such that an outer portion of the button or pad structure 650 pivots relative to an inner portion of the button or pad structure 650 about the pin structure 619 along the axis labeled A-A. FIG. 6H also illustrates the orientation of the optical waveguide 654 in the device 602 and shows the waveguide 654 supported by a flexible circuit substrate structure 656. Specifically, the optical waveguide 654 extends generally along an axis labeled B-B, with an optical source 658 provided at one end of the waveguide 654 and an optical detector 660 provided at an opposite end of the waveguide 654. Also shown in FIG. 6H is the rechargeable battery 628 and the dual-pin port device 611 for connecting a charging device to the device 602 to charge the battery 628.

Referring now to FIGS. 6I1-5, there is provided a series of drawings to further illustrate the design of the wrist-worn system 601 shown in FIGS. 6A-6H. Specifically, there is shown a wrist-worn system 601 including the monitoring device 602 connected at one side to a first strap portion 603a and at a second, opposite side to a second strap portion 603b. In this embodiment of a wrist-wearable band, hook-and-loop fastening structures such as those provided with VELCRO® brand products are utilized. As such, the second strap portion 603b) includes first and second hook-and-lock fastening structures 606a, 606b, provided on a top surface of a strap substrate structure 606c. As such, a distal end of the second strap portion 603b may be advanced through an opening in a buckle 604 provided at a distal portion of the first strap portion 603a, and then looped around so that the two hook-and-loop fastening structures 606a, 606b may be mated face-to-face against one another for fastening. Then if desired, the band portions 603a, 603b may be easily adjusted (e.g., tightened or loosened) to achieve a desired hold-down force of the button or pad structure 650 against the surface of the skin adjacent the radial artery.

FIG. 6I4 shows a longitudinal cross-section of the wrist-worn system 601 along the cross-section A-A of FIG. 6I1. Further detail of the FIG. 6I4 cross-section focusing on the monitoring device 602 is shown in FIG. 6I5. Generally, in a first portion 602a of the monitoring device 602 (at left in FIG. 6I5) there is provided the electro-optical sub-system (albeit only the optical waveguide 654 is shown in the cross-section), the button or pad structure 650, the leaf spring 652, and some but not all of the electronics. In a second portion 602b of the monitoring device 602, there is provided the battery 628 and the rest of the electronics. A single structure flexible circuit substrate 656 is provided that resides in both portions 602a, 602b, of the monitoring device 602, as is illustrated in more detail below (for example, in FIGS. 6J and 6N1-7).

FIG. 6I5 also shows one example connection configuration for the device 602 to connect to the band portions 603a, 603b. The device 602 is provided with four connecting structures, two of which 643b, 643d are shown in FIG. 6I5. Pins 646a, 646b are provided as shown to secure band portions 603a, 603b, in a manner described in more detail below in connection with FIG. 6L1. Additional structures labelled in FIG. 6I5 will be described below with reference to other figures.

FIG. 6J shows an exploded view of the monitoring device 602 to illustrate the device's constituent parts. As illustrated in FIG. 6J, the monitoring device 602 includes two external housing components—a bottom housing component 631 and a top housing component 632—adapted to be connected to one another to form the external housing 615 for the device 602. Housing component 631 is referred to herein as a “bottom” housing component, despite being shown at the top of FIG. 6J, given that when the device 602 is worn as intended, the bottom housing component 631 would be adjacent the surface of the wrist, whereas the “top” housing component 632, when in use, would be furthest from the subject's skin. An internal sensing system 637, which includes a fulcrum component 634 and an electro-optical motion sensing system 635, resides within an internal chamber formed by the two housing components 631, 632, when connected to one another to form the device external housing 615. The electro-optical motion sensing system 635 is, in part, carried by, and engaged against, the fulcrum component 634. A skin interfacing system 636 is shown in FIG. 6J only in part and assembled with the bottom housing component 631. The skin interfacing system 636 includes the button or pad structure 650 that is configured on one side (that is, the skin interfacing surface 622) to bear against a surface of a subject's skin when in use, and on an opposite side to bear against the side of an optical waveguide 654 and/or a flexible circuit substrate 656 underlying the optical waveguide 654. The flexible circuit substrate 656 and the optical waveguide 654 are part of the electro-optical motion sensing system 635. The button or pad structure 650 is assembled with the bottom housing component 631 so the button or pad structure 650 is positioned near an opening 655 in the bottom housing component 631.

In more detail, the bottom housing component 631 has, in this embodiment, a generally cuboid shape with a slight inwardly curved shape corresponding generally to the curvature of the wrist against which it is worn. The bottom housing component 631 has the following structures: (1) a generally rectangular but slightly inwardly curved bottom wall 661 comprising first and second portions 661a, 661b; (2) two generally flat side walls 662a, 662b that are curved complementary to the curvature of the bottom wall 661 (the “side” walls referring to a side of housing component 631 that extends generally parallel with the length of the bands 603a, 603b, shown for example in FIG. 6E); and (3) two generally flat, rectangular end walls 663a, 663b (the “end” walls referring to a side of the housing component 631 that also is adjacent to, and connects with, the bands 603a, 603b). The side and end walls 662, 663 form a generally rectangular opening (not shown in FIG. 6J, in that the opening is on the underside of housing component 631 as oriented in FIG. 6J) that is opposite the bottom wall 661. The previously mentioned circular opening 655 is provided in the bottom wall 661, generally in a corner of the bottom wall 661. The circular opening 655 is positioned in the bottom wall 661 so that the generally cylindrically shaped button or pad structure 650 of the skin interfacing system 636 is aligned therewith. As such, the button or pad structure 650 is permitted to extend through the circular opening 655 so that, when in use as intended, the skin contacting surface 622 of the button or pad structure 650 makes contact with the surface of the skin of a subject adjacent the radial artery.

The top housing component 632 has, in this embodiment, a generally cuboid shape with the same footprint as the bottom housing component 631 to which the top housing component 632 is mated to form the device external housing 615. The top housing component 632 has a slight outwardly curved shape corresponding generally to the inward curvature of the top housing component 631 (which again, corresponds generally to the curvature of a wrist against which the device 602 is worn). Top housing component 632 has the following structures: (1) a generally rectangular but slightly outwardly curved top wall 664 comprising first and second portions 664a, 664b, and having a similar size to the generally rectangular bottom wall 661; (2) two generally flat side walls 665a, 665b that are curved complementary to the curvature of the top wall 664 (the “side” walls referring to a side of housing component 632 that extends generally parallel with the length of the bands 603a, 603b); and (3) two generally flat, rectangular end walls 666a, 666b (the “end” walls referring to a side of the housing component 631 that also is adjacent the bands 603a, 603b). The side and end walls 665, 666 form a generally rectangular opening that is opposite the top wall 664. Exposed top edges 667 of the side and end walls 665, 666 of the top housing component 632 are sized and configured to mate with exposed bottom edges (not shown in FIG. 6J) of the top side and end walls 662, 663 of the bottom housing component 631. Connection of the bottom housing component 631 to the top housing component 632 may be provided by snap-fit mechanism, gluing, or any suitable fixation means.

The top and bottom walls 664, 661 are each generally divided into two portions, namely, first top and bottom wall portions 664a, 661a, and second top and bottom wall portions 664b, 661b. In the top wall 664, a dividing structure 668 is provided on an inner surface of the top wall 664, extending inwardly from an inner surface of the first side wall 665b and along a dividing line between the two top wall portions 664a, 664b, as shown in FIG. 6J. Generally, the first top and bottom wall portions 664a, 661a each cover roughly two-fifths of the area of their respective top and bottom walls 664, 661, whereas the second top and bottom wall portions 664b, 661b each cover the remaining roughly three-fifths of the area of their respective top and bottom walls 664, 661. The first top and bottom wall portions 664a, 661a define the first portion 602a of the device 602 (first portion 602a defined in FIG. 6I5) and define an internal chamber therebetween in which resides the skin interfacing system 636 (including the leaf spring 652 and the button or pad structure 650), all of the electro-optical components, and most of the fulcrum component 634. The second top and bottom wall portions 664b, 661b define the second portion 602b of the device 602 (second portion 602b defined in FIG. 6I5) and define an internal chamber therebetween in which resides much of, but not all of, the electronics and the battery 628.

The charging port 611 is assembled with the flexible circuit substrate 656 of the motion sensing system 635, at a location that is adjacent the bottom and top housing component side walls 662a, 665a, such that the port 611 extends through an opening formed by corresponding notches 638a, 638b in the bottom and top housing component side walls 662a, 665a. When assembled, two charging leads 639 of the battery 628 make electrical contact with two leads of the two-lead charging port 611. A cylindrical on-off switch spacer 616 is provided and positioned on top wall portion 664a adjacent the on-off button 605 provided on the outer surface of the top housing component 632 (not shown in FIG. 6J but shown for example in FIG. 6A).

Turning now to FIGS. 6K1-5, additional views of the top housing component 632 are shown. FIG. 6K1 is an outside view of the top housing component 632, showing specifically an outer portion of the housing 615 that would be seen if the wrist-worn monitoring device 602 were being worn as intended, as shown in FIG. 6A. FIG. 6K4 shows a view from the inside of the top housing component 632. As shown in FIG. 6K1, the on/off button 605 is provided at a generally central location of a first surface plate 640a that is affixed to an outer surface of the first top wall portion 664a. For connection of that button 605 to circuitry within the device 602, a circular opening 641 is provided in the first top wall portion 664a (and the cylindrical on-off switch spacer 616 shown in FIG. 6J is provided in the circular opening 641), at a generally central location corresponding to the location of the button 605 on the opposite side of the wall portion 664a. For the LED indicator 607 (shown in FIG. 6A, for example), corresponding circular openings 642a, 642b are provided in the first surface plate 640a and in the first top wall portion 664a, thus allowing the LED indicator 607 to protrude therethrough from the inside of the device 602 and thus be seen by a user. The “left hand” indicator 609 may be provided on the first surface plate 640a, as shown in FIG. 6K1. A second surface plate 640b may also be provided on an outer surface of the second top wall portion 664b, as shown in FIG. 6K1, for decorative reasons or scratch resistance.

FIG. 6K2 is an end side view of the top housing component 632 facing second end wall 666b; FIG. 6K3 is a side view facing first side wall 665a; FIG. 6K5 is an end side view facing the first end wall 666a. FIGS. 6K6-6K8 are perspective views of the top housing component 632, with FIG. 6K6 showing its upper side, FIG. 6K7 showing its underside, and FIG. 6K8 being an exploded view showing the individual parts of the top housing component 632. The outwardly curved nature of the top housing component 632 is illustrated in the side view of side wall 665a in FIG. 6K3 as well as in the perspective views of FIGS. 6K6-6K8. Owing to the outwardly curved nature of the top housing component 632, FIG. 6K2 shows not only the second end wall 666b, but also shows a portion of the curving second surface plate 640b also shown in FIGS. 6K1 and 6K3. Additionally, FIG. 6K5 shows not only the first end wall 666a, but also shows a portion of an inside surface of the second end wall 666b on the opposite end of the top housing component 632. The previously described notch 638b for the charging port 611 that is formed in the first side wall 665a is shown in FIGS. 6K3 and 6K4, and the previously described dividing structure 668 provided on the top wall 664 and abutting the second side wall 665b is shown in FIG. 6K4.

Turning next to FIGS. 6L1-6L7, further detail is provided for the bottom housing component 631 and the skin interfacing system 636 (the latter of which includes the leaf spring 652 and the button or pad structure 650). FIG. 6L1 is an exploded view showing the skin interfacing system 636 separate from the bottom housing component 631. The skin interfacing system 636 includes the thin rectangular-shaped leaf spring 652 and a connected button or pad structure 650. The generally cylindrically shaped button or pad structure 650 is sized and configured to extend through the circular opening 655 provided in the first bottom wall portion 661a. The location of the circular opening 655 is generally to one side of the first bottom wall portion 661a, as shown in FIG. 6L1, so that the button or pad structure 650 is suitably positioned for placement on the skin over the radial artery when worn on a left wrist of a user, as intended. The button or pad structure 650 extends through the housing circular opening 655 with its skin facing surface 622 facing outward (as shown also in FIGS. 6L4-6L5 and 6L7), so that the skin facing surface 622 may be placed in contact with a surface of the wearer's skin adjacent the radial artery. Further detail of the skin interfacing system 636 including the leaf spring 652 and the button or pad structure 650 is provided below in connection with FIGS. 6L5-6L7 as well as FIGS. 6M1-6M5.

As further shown in FIG. 6L1, the opposing pair of end walls 663a, 663b of the bottom housing component 631 include four band connecting structures 643a-d, with two of the structures 643a-b for connecting the device 602 to the first strap portion 603a and the other two of the structures 643c-d for connecting the device 602 to the second strap portion 603b (see FIG. 6I5). The strap connecting mechanism may include two longitudinally compressible pin devices 646a, 646b (shown in FIG. 6I5), each of which extends between indentions in the inner sides of a corresponding pair of connecting structures 643a-b, 643c-d and through a channel formed at proximal ends of the strap portions 603a, 603b, to be able to connect and release each of the two strap portions 603a, 603b from the monitoring device 602. It will be understood that many other connection structures may be provided as alternatives to the pin-type as shown.

FIG. 6L2 is a perspective view of the bottom housing component 631 showing its inner design. A leaf spring containing structure 644 is provided within the bottom housing component's first bottom wall portion 661a. The leaf spring containing structure 644 is configured to form a horizontally extending channel 696 corresponding generally to the width of the leaf spring 652. As such, the leaf spring 652 may be slid into the leaf spring containing structure's horizontal channel 696, as shown in FIGS. 6L6 and 6L7, and may be secured to the leaf spring containing structure 644 by suitable means such as glue. When the leaf spring 652 is properly positioned and secured within the containing structure 644, the leaf spring 652 extends from within the horizontal channel 696 to a location in the vicinity underlying the circular opening 655, at which location the leaf spring 652 is affixed to the button or pad structure 650, as shown for example in FIG. 6L7. As is also shown in FIG. 6L2, a dividing structure 645 is provided on an inner surface of the bottom wall 661, along a border between the first bottom wall portion 661a and the second bottom wall portion 661b. The dividing structure 645 extends between the two side walls 662a and 662b.

FIGS. 6L3-6L7 illustrate the skin interfacing system 636 (including the leaf spring 652 and the button or pad structure 650) and how that system 636 is assembled with the bottom housing component 631. First, FIG. 6L3 is a bottom-side view directly facing an underside surface of the bottom housing component 631 with the assembled skin interfacing system 636. In other words, this view shows the skin-facing side of the monitoring device 602. In the view of FIG. 6L3, the skin interfacing system 636 is largely on the opposite side of the housing component 631 and thus largely obstructed from view. This view also shows the skin facing surface 622 of the button or pad structure 650 positioned within the perimeter of the circular opening 655 provided in the first bottom wall portion 661a. A small portion of the leaf spring 652 is also seen through the circular opening 655, extending to the side of button or pad structure 650.

FIG. 6L4 is a side view of the bottom housing component 631 with assembled skin interfacing system 636, directly facing the first bottom housing side wall 662a. FIG. 6L4 illustrates the generally inwardly curved design of the bottom housing component 631, to accommodate its positioning on a subject's wrist. Reference number 617, as discussed previously in connection with FIG. 6F, indicates a surface that would typically bear against the skin of a user when the device 602 is worn on the wrist of a subject as shown in FIGS. 6A and 6B. As illustrated in FIG. 6L4, the skin facing surface 622 of the button or pad structure 650 extends beyond (that is, below) a bottom surface of the first bottom wall portion 661a, thus allowing forces present on the surface of the skin adjacent an artery to press the button or pad structure 650 inward.

FIGS. 6L5-6L7 illustrate further detail as to how the skin interfacing system 636 is assembled with the bottom housing component 636. FIG. 6L5 is a cross-sectional view taken along plane A-A labeled in FIG. 6L3, parallel with the two sides 662a, 662b of the housing component 631; FIG. 6L6 is an underside view, directly facing the underside of the bottom housing component 631 with assembled skin interfacing system 636; and FIG. 6L7 is a cross-sectional view taken along plane B-B labeled in FIG. 6L3. As shown in FIGS. 6L6 and 6L7, one end portion of the leaf spring 652 is positioned within the horizontal channel 696 of the leaf spring containing structure 644, and an opposite end of the leaf spring is affixed to the button or pad structure 650.

FIGS. 6M1-6M5 illustrate the design of just the skin interfacing system 636. Specifically, FIG. 6M1 is a view directly facing its skin facing side. In other words, this view shows the side that would face the user's skin. FIG. 6M3 is a view on the opposite side of that shown in FIG. 6M1. FIGS. 6M2 and 6M4 are cross-sectional views along respective planes A-A and B-B shown in FIGS. 6M1 and 6M3. FIG. 6M5 is an exploded view to illustrate the individual parts of the skin interfacing system 636.

With reference to FIGS. 6L6-6L8 and FIGS. 6M1-6M5, it is seen that the button or pad structure 650 in this embodiment includes two main components that are pivotably connected with a pin structure 619. The first main component is an inner button part 694 having a generally cylindrical shape. The inner button part 694 is oriented such that, as shown with reference to FIGS. 6L7 and 6M5, the longitudinal axis of its cylindrical shape will, when the skin interfacing system 636 is assembled with the bottom housing component 631, extend (i) parallel with the housing component's bottom wall portion 661a, and (ii) parallel with the housing component's side walls 662a, 662b. The inner button part 694 includes the waveguide and/or substrate contacting surface 651, as labeled in FIGS. 6L5, 6L7, 6M2, 6M4 and 6M5. The second main component of the button or pad structure 650 is an outer button part 695 also having a generally cylindrical shape. The outer button part 695 is oriented such that the longitudinal axis of its cylindrical shape extends (i) perpendicular to the longitudinal axis of the cylindrical shape of the inner button part 694 (as shown for example in FIG. 6M5), and (ii) parallel with both the housing component's side wall portion 662a and end wall portion 663a (as shown in FIGS. 6L5 and 6L7). The outer button part 695 includes the button or pad structure's outer skin-contacting surface 622, as shown for example in FIGS. 6L5, 6L7, 6M2, 6M4 and 6M5.

As illustrated best in FIGS. 6L8 and 6M2, the outer button part 695 in this embodiment may be shaped so that its skin contacting surface 622 is generally flat with a beveled periphery, and/or may have a ramping design such that the outer button part 695 is larger on one side of the pivotable connection point (the side nearer the device periphery) than on the opposite side of the pivotable connection point. As such, the design of the outer button part 695 tends to face generally inward although is pivotable inward and outward, and as such may assist in maintaining better contact between the skin contacting surface 622 of the button or pad structure 650 and the surface of the subject's skin adjacent an artery. In some implementations, the skin contacting surface 622 may have other profiles and configurations, for example, a domed surface as opposed to the generally flat surface with beveled edges as shown in FIGS. 6L8 and 6M2.

The outer button part 695 is pivotally connected with the inner button part 694, with the inner button part 694 fitting in part within the outer button part 695. This is possible because, as illustrated in FIGS. 6M3-6M5, the profile of the outer button part 695 entirely encompasses the profile of the inner button part 694, or in other words, the entire length of the horizontally extending inner button part 694 fits within the circumferential extent of the outer button part 695. Additionally, the distance between longitudinal ends of the inner button part 694 is shorter than the distance between opposing side walls 649a, 649b of the outer button part 695; as such, a portion of the inner button part 694 including longitudinal borehole 647 for pin structure 619 fits entirely within a volume between the outer button part's opposing side walls 649a, 649b. The outer button part's side walls 649a, 649b each have a borehole 648a, 648b positioned on corresponding side walls 649a, 649b so that the pin structure 619 is able to extend through the sidewall boreholes 648a, 648b and also through the inner button part's longitudinal borehole 647, and as such the pivotable connection between the outer button part 695 and the inner button part 694 is provided.

The leaf spring 652 is fixedly connected to the lower button portion 694, as is illustrated for example in FIGS. 6M2 and 6M4. Specifically, an end portion of the leaf spring 652 (namely, the end portion that is opposite the end portion connected to the leaf spring containing structure 644 as shown in FIGS. 6L6-6L7) is inserted into a horizontal channel 698 that extends axially and entirely through the lower button portion 694 (see, e.g., FIGS. 6M4 and 6M5), and affixing the leaf spring 652 to the insides of the channel 698 by gluing or some other suitable fixation means.

As illustrated, the outer button part 695 is configured to pivot relative to the inner button part 695 by means of the pin structure 619 extending through the outer button part side walls 649a, 649b and longitudinally through the inner button part 694, as shown for example in FIGS. 6M4 and 6H. The pin structure 619 is retained within boreholes 647, 648a, and 649b by virtue of the upper button portion side walls 649a, 694b being contained within button structure containing side walls 699 (shown in FIG. 6L2) formed in the bottom housing component 631 at a location where the button or pad structure 650 is positioned when assembled. The outer button part 695 pivots about an axis of the pin structure 619 such that, when the wrist wearable device is being worn as intended, such pivoting axis is oriented to extend perpendicularly to the length of the lower arm and wrist. With reference now to FIGS. 6A-B and 6F-G, in some cases a suitable or optimal location to position the skin contacting surface 622 of the button or pad structure 650 against the skin surface adjacent a radial artery may be near the wrist where the diameter of the wrist/forearm starts to increase (that is, extend outwardly). As such, it may be appreciated that the pivotable configuration of the two-part button or pad structure 650 and/or the ramped profile of the upper button portion 695 may assist in maintaining a desired contact of the skin contacting surface 622 to the surface of the skin adjacent the radial artery for users with a variety of different wrist anatomies.

FIGS. 6N1-8 illustrate in further detail the internal sensing system 637 previously shown in the exploded view of FIG. 6J. The internal sensing system 637 comprises the electro-optical motion sensing system 635 (portions of which are shown in more detail in FIGS. 6O1-3) assembled with the fulcrum component 634 (of which more detail is shown in FIGS. 6P1-3). More specifically, FIG. 6N1 is a perspective view of the internal sensing system 637, and FIG. 6N2 is an exploded view of the internal sensing system 637 showing its separate parts. FIGS. 6N3, 6N4 and 6N5 are, respectively, top, side and end views of the internal sensing system 637. FIGS. 6N6-7 and 6N8 are cross-sectional views along, respectively, planes A-A and K-K defined in FIG. 6N3. FIGS. 6O1-3 show further detail of a sub-assembly 608 included in the electro-optical motion sensing system 635 shown in FIGS. 6N1-8 (the sub-assembly 608 being all of the motion sensing system 635 except the optical waveguide 654 and detector 660 components). FIGS. 6P1-3 and 6Q1-3 show two parts 634a, 634b that make up the fulcrum component 634 shown in FIGS. 6N1-8.

Referring first to FIGS. 6N1 and 6N2, the fulcrum component 634 and the electro-optical motion sensing system 634 are assembled such that a first lengthwise portion 654a of the optical waveguide 654 (roughly one-half of the waveguide's entire length, which portion 654a interfaces with the optical detector 660) remains stationary during operation of the monitoring device 602, whereas a second lengthwise portion 654b of the optical waveguide 654 (the remaining roughly one-half of the waveguide's entire length, which portion 654b interfaces with the optical source 658) is permitted to flex during operation of the monitoring device 602. To achieve that functionality, the first optical waveguide portion 654a is mounted upon a first portion 656a of the flexible substrate structure 656 that remains stationary during use by virtue of being positioned on a rigid top ramping surface 682 (see FIG. 6N2), and the second optical waveguide portion 654b is mounted upon a second portion 656b of the flexible substrate structure 656 that is permitted to flex up and down depending on forces applied against a top-side surface of the second optical waveguide portion 654b and/or the corresponding flexible substrate portion 656b upon which the second optical waveguide portion 654b is mounted. Such forces are applied in a manner described previously, namely, by the contacting portion 651 of the button or pad structure 650 (see FIG. 6M2) bearing against a side of the second optical waveguide portion 654b and/or the corresponding flexible circuit substrate portion 656b upon which the second waveguide portion 654b is mounted, the bearing force being responsive to forces present on the skin surface adjacent an underlying artery during use of the device 602 as intended. As such, optical power modulation (OPM) operation is enabled in a manner discussed previously.

As shown in FIG. 6N1, the electro-optical motion sensing system 635 in this embodiment, of which the sub-assembly 608 shown in FIGS. 6O1-3 is a part, includes all of the electro-optical components and various discrete and integrated electronic components. In FIGS. 6O1-3, the sub-assembly 608 is shown with the flexible circuit substrate structure 656 and mounted components in a “flattened out” configuration, that is, before assembly of the sub-assembly 608 with the fulcrum component 634. In particular, FIG. 601 is a view of the flattened-out sub-assembly 608 from an underside perspective with reference to the orientation shown in FIG. 6N2, whereas FIG. 603 is from a top-side perspective with reference also to FIG. 6N2 (that is, the opposite side of the underside shown in FIG. 601). FIG. 602 shows a side view of the sub-assembly 608 shown in FIGS. 6O1 and 6O3, directly facing a side 618 that is shown at the bottom of the sub-assembly 608 as labeled in FIGS. 6O1 and 6O3.

Referring briefly to FIGS. 6O1 and 6O3 and also FIG. 6N2, the flexible circuit substrate structure 656 is provided generally in an “L” shape. A leg or extension portion 612 of the L-shaped substrate structure 656 (which portion 612 includes the previously mentioned first, stationary portion 656a and the second, flexing portion 656b) has mounted thereon the electro-optical components comprising the optical source (e.g., an LED) 658, the optical waveguide 654, and the optical detector 660, as illustrated in FIGS. 6N1 and 6N2. Of these three electro-optical components, only the LED 658 is shown as having been provided with the sub-assembly 608 shown in FIGS. 6O1-6O3. The waveguide 654 and detector 660 are assembled with sub-assembly 608. Referring to FIGS. 6N1 and 6N2, the optical waveguide 654 and optical detector 660 may be mounted on the sub-assembly 608 of FIGS. 6O1-6O3 after the sub-assembly 608 is first assembled with a first fulcrum component part 634a and before a second fulcrum component part 634b is connected to the first fulcrum component part 634a. The L-shaped flexible circuit substrate structure 656 also includes a main portion 614 including all of the remaining portion of the substrate structure 656 aside from the leg or extension portion 612. The main portion 614 of the substrate structure 656 has mounted thereon substantially all of the discrete and integrated electronic components.

Generally, the electro-optical motion sensing system 635 and the fulcrum component 634 are assembled so that the main portion 614 of the flexible circuit substrate 656 and associated mounted components reside in part under, and in part to the side of, the fulcrum component 634, as illustrated in FIGS. 6N1-2. The leg or extension portion 612 of the substrate structure 656 during assembly may be flexed upward and “wrapped around” a fulcrum body 681 of the fulcrum component 634, and positioned vis-à-vis the fulcrum body 681 so that the first, stationary portion 656a of the flexible circuit substrate 656 rests upon a rigid top ramping surface 682 of the fulcrum body 681 and the second, flexing portion 656b of the flexible circuit substrate structure 656 extends beyond a side face 685 of the fulcrum body 681 and thus is permitted to flex downward and then back to a resting position during OPM operation of the monitoring device 602 as previously described.

The fulcrum component 634 (with which the sub-assembly 608 of FIG. 601-3, optical waveguide 654 and optical detector 660 are assembled) will now be described in detail, with reference to FIGS. 6P1-6P3 and 6Q1-6Q3. FIGS. 6P1-6P3 show the second fulcrum component part 634b, and FIGS. 6Q1-6Q3 the first fulcrum component part 634a, both in perspective views. Specifically, FIGS. 6P1 and 6Q1 show the two fulcrum component parts 634b, 634a in an orientation also shown in FIGS. 6N1 and 6N2. FIGS. 6P2 and 6Q2 show the two fulcrum component parts 634b, 634a rotated 180° about a vertical axis as compared to FIGS. 6P1 and 6Q1 (for example, to show what is on a backside of the parts 634b, 634a in the orientation of FIGS. 6P1 and 6Q1). FIGS. 6P3 and 6Q3 show the two fulcrum component parts 634b, 634a “flipped up” 900 compared to FIGS. 6P2 and 6Q2 (for example, to show what is on an underside of the parts 634b, 634a in the orientation of FIGS. 6P1-6P2 and 6Q1-6Q2).

The first and second fulcrum component parts 634a, 634b are designed with structures that mate together in side-by-side fashion to form the assembled fulcrum component 634 as shown in FIG. 6N1. To provide for this, as shown for example in FIGS. 6P1-3 and 6Q1-3, the first fulcrum component part 634a has a horizontal slot 620 extending inwardly from an inner side surface 621 of the first fulcrum component part 634a, which horizontal slot 620 is positioned to align with a complementary horizontally extending extension 623 extending outwardly from an inner side surface 624 of the second fulcrum component part 634b as illustrated in FIG. 6N1.

Referring to FIGS. 6N1, 6P1, and 6Q1, the fulcrum component 634 generally comprises: (1) a fulcrum structure 672 that includes a fulcrum portion 672a of the first fulcrum component part 634a and all of the second component fulcrum component part 634b; and (2) a chamber dividing structure 625 that includes a generally horizontally oriented dividing wall 626. The dividing wall 626 divides a portion of an internal chamber within the housing components 631, 632 into (1) a first chamber portion within which the optical waveguide portion 654b and corresponding flexible circuit substrate structure 656b are permitted to flex to their full extent during optical power modulation operation; and (2) a second chamber portion within which a portion of the flexible circuit substrate structure 656 (specifically, a portion of the main substrate portion 612) and electronics mounted thereon are positioned to reside. In the orientation shown in FIGS. 6N1-2 and 6Q1, the first chamber portion in which the optical waveguide portion 654b and corresponding flexible circuit substrate structure 656b are positioned is above the dividing wall 626, and the second chamber portion in which a portion of the main substrate structure 612 and electronics mounted thereon are positioned is below the dividing wall 626.

The fulcrum component 634 in this embodiment has an overall size and generally cuboid shape so that the fulcrum component 634 is housed mainly within an internal chamber of the first portion 602a of the device 602 (see FIG. 6I5 defining the first portion 602a). As such, and referring to FIG. 6J, it is seen that the fulcrum component 634 upon assembly becomes located mainly between the first bottom wall portion 661a and the first top wall portion 664a. That said, although mainly located within the first portion 602a, the fulcrum component 634 in this embodiment is not solely located there, but rather extends into the second portion 602b of the device 602. In particular, the chamber dividing structure 625 of the fulcrum component 634 in this embodiment extends into the second portion 602b, or in other words, into a chamber located between the second bottom wall portion 661b and the second top wall portion 664b (see, e.g., FIGS. 6N1-3 and 6N5).

The dividing wall 626 in the present wrist-worn embodiment has two portions 626a, 626b, as can be seen well in FIG. 6Q2. Referring to FIG. 6I5 defining the first and second device portions 602a, 602b of the monitoring device 602, the first dividing wall portion 626a is housed within the first device portion 602a (that is, between top housing component's first top wall portion 664a and bottom housing component's first bottom wall portion 661a), and the second dividing wall portion 626b is housed within the second device portion 602b (that is, between top housing component's second top wall portion 664b and bottom wall component's second bottom wall portion 661b). The second dividing wall portion 626b lies in a plane that is angled slightly relative to the first dividing wall portion 626a (the angling being upward with respect to the FIG. 6N1 orientation). The upward angling is shown for example in the side and cross-sectional views of FIGS. 615, 6N5, 6N8, as well as in the perspective views of FIGS. 6N1-6N2 and 6Q1-6Q3. Such angling of the second dividing wall portion 626b relative to the first dividing wall portion 626a is present and consistent with the housing inner chamber shape as constrained by the outward curvature of the top housing component 632 and the inward curvature of the bottom housing component 631, which curvatures make the device 602 appropriately shaped to be worn about and against the wrist.

The fulcrum structure 672 has, on opposite sides, two generally flat side walls 671a, 671b. Side wall 671b is rectangular. Side wall 671 is L-shaped, as shown in FIGS. 6Q2 and 6Q3. Vertically, the side walls 671a, 671b extend from co-planar top surfaces defining in part a top surface 673 of the fulcrum component 634, to co-planar bottom surfaces defining in part a bottom surface 674 of the fulcrum component 634 (with “top” and “bottom” here being defined in the orientation as shown in FIGS. 6P1 and 6Q1, although it will be appreciated that the “top” side here is nearer the user's skin surface than the “bottom side,” when the device 602 is worn). The bottom surface 674 of side wall 671a (shown in FIG. 6Q3) has an outside side surface (i.e., the side surface shown in FIG. 6Q3 that lies abutted against a side surface of the top housing component's dividing structure 668 (shown for in FIG. 6K7). Specifically, the side surface of the chamber dividing structure 668 against which side wall 671a abuts is the side surface facing the first portion 602a of the device 602 (that is, the side surface shown in FIGS. 6J and 6K7). Now referring back to FIG. 6Q3, another short inner end wall 671e lies adjacent to, and extends the entire length of, an inside end of a lower-L extension of the first side wall 671a. Short inner end wall 671e has a bottom surface also lying in the plan of the fulcrum component's bottom surface 674, as shown in FIG. 6Q3. In addition, the short inner end wall 671 also abuts against the chamber dividing structure 688, specifically abutting against an inner end surface of the chamber dividing structure (shown in FIGS. 6J and 6K7).

The chamber dividing structure 625 has a generally flat, rectangular side wall 671c adjacent to and extending co-planar with the fulcrum structure side wall 671a. The side wall 671c also extends downward from, and extends along the entire length of, a side end edge of the horizontal dividing wall 626. The chamber dividing structure 625 also has a generally rectangular end wall 671d that is adjacent a corner end edge of the side wall 671c, which corner end edge is opposite the lengthwise end of the side wall 671c that is adjacent the fulcrum structure's side wall 671a. The chamber dividing structure end wall 671d also extends downward from, and extends along the entire length of, a top end edge of the horizontal dividing wall 626. The bottom surfaces of the side wall 671c and end wall 671d both lie generally in a common plane with bottom surfaces of the fulcrum structure's side walls 671a, 671b, as can be seen in FIGS. 6Q3 and 6N3. As such, the bottom surfaces of side wall 671c and end wall 671d also define in part the bottom surface 674 of the fulcrum component 634. With reference to FIG. 6J as well as FIGS. 6P1-3 and 6Q1-3, upon assembly of the fulcrum component 634 with the bottom and top housing components 671, 672, the top surface 673 of the fulcrum component 634 will abut against an inner surface of the bottom housing component's leaf spring containing structure 644 (see FIG. 6L2), whereas the bottom surface 674 of the fulcrum component 634 will abut against an inner surface of the top housing component's top wall portion 664a and also a portion of the top housing component's second top wall portion 664b. As such, it can be seen that upon assembly the fulcrum component 634 becomes “sandwiched” between the bottom and top housing components 631, 632 mainly in the first portion 602a of the monitoring device 602 although also in a portion of the second portion 602b of the monitoring device 602. In particular, the dividing wall second portion 626b and a portion of end wall 671d adjacent dividing wall portion 626b reside in the device's second portion 602b.

Referring to FIGS. 6N2 and 6Q1-3, the first fulcrum structure portion 672a includes a ramped fulcrum body 681. The top ramping fulcrum surface 682 of the fulcrum body 681 (shown best in FIGS. 6Q1-2) serves as a fulcrum for the optical waveguide 654. As described previously, the optical waveguide 654 is provided on the flexible substrate surface 656. The first lengthwise portion 654a of the optical waveguide 654 is supported by the fulcrum body top surface 682, and the second lengthwise portion 654b of which optical waveguide 654 extends beyond the fulcrum body top surface 682 and thus is able to flex in response to a force applied to its side by the button or pad structure 650 during operation of the monitoring device 602. The ramped fulcrum body 681 also has a generally flat, and recessed, bottom surface 679. The recessed nature of the bottom surface 679 forms, together with the top housing component's top wall portion 664a (see FIGS. 6J and 6K4), a chamber within which mounted electronics may reside upon assembly of sub-assembly 608 including the flexible substrate structure 656 and mounted electronics with the first fulcrum component part 672a.

Referring now to the cross-section of FIG. 6N7, the ramped fulcrum body's top fulcrum surface 682 is opposite the generally flat, recessed bottom surface 679, and referring to FIG. 6N1 extends between, and has a side-to-side orientation that is perpendicular with, the two fulcrum component side walls 671a, 671b. In other words, the side-to-side orientation of the top ramping fulcrum surface 682 is generally parallel to the fulcrum component's bottom surface 674. Referring back to FIG. 6N7, it is seen that the top fulcrum surface 682 rises or elevates (ramps up) from a low-end position 683 that is adjacent to an end wall 610 of the fulcrum body 681 (which in turn is adjacent the fulcrum body's recessed bottom surface 679) to a high-end position 684 adjacent the fulcrum structure's inner side face 685 (when viewing the top ramping fulcrum surface 682 from left to right in the perspective of FIG. 6N7). The top fulcrum surface 682 may be said to be “rounded off” in that its grade (steepness) tapers near the inner side face 685 of the fulcrum body 681. Specifically, the grade of the surface 682 is initially steep, at about a 35-40 degree angle, at the low-end position 683, and then tapers such that the grade eventually becomes nearly horizontal at the high-end position 684 of the top fulcrum surface 682. The high-end position 684 of the top ramping fulcrum surface 682 is adjacent the side face 685 of the ramped fulcrum body 681, which is also the side face 685 of the entire fulcrum structure 672, as shown in FIGS. 6N2 and 6Q1. In final assembly, the first portion 656a of the flexible circuit substrate 656, having an optical detector 660 provided thereon along with a first portion 654a of the optical waveguide 654, is supported thereunder by the top ramping fulcrum surface 682 (optionally with a leaf spring 697 in part lying therebetween).

Referring again to FIG. 6N1, the fulcrum structure 672 also includes two inwardly extending arms 687a, 687b, each extending inwardly from, perpendicular to, and integral with respective ones of the fulcrum structure's two opposing side walls 671a, 671b. Top surfaces of the inwardly extending arms 687a, 687b make up a portion of the generally flat fulcrum component top surface 673, which top surface 673 as described previously is positioned against or near an inner surface of the bottom housing component's leaf spring containing structure 644 (see FIG. 6L2). Located opposite of the top surface 673, each of the inwardly extending arms 687a, 687b has a respective ramping underside surfaces 689a, 689b (see FIGS. 6Q1 and 6P3) with a shape profile that is generally complementary to, and faces, the rounded-off ramp shape profile of the ramp structure's top ramping fulcrum surface 682 (see FIG. 6N2). A small horizontal gap or slot 690 is provided between the inwardly extending arm underside surfaces 689a, 689b and the ramp structure's top ramping fulcrum surface 682. The small horizontal gap or slot 690 provides a space for positioning, during assembly, the first substrate portion 656a of the flexible circuit substrate structure 656, in a manner such that the first substrate structure portion 656a resides between (and may become effectively “sandwiched” between) the top ramping fulcrum surface 682 and the inwardly extending arm underside surfaces 689a, 689b (see FIGS. 6N1 and 6N7).

Additionally, when the two fulcrum component parts 634a, 634b are assembled together, a small vertical gap 688 may be provided between the two facing distal ends of the inwardly extending arms 687a, 687b (see FIG. 6N1 and also FIG. 6N3). The small vertical gap 688 provided between the inwardly extending arms 687a, 687b may facilitate assembly in some implementations. For example, the optical waveguide 654 may be advanced between the vertical gap 688 and the waveguide portion 654a placed upon a surface of the first flexible circuit substrate portion 656a already positioned upon the fulcrum structure's top ramping fulcrum surface 682. This may be useful in implementations in which the two fulcrum component parts 634a, 634b are assembled together prior to placement of the optical waveguide 654 on the substrate structure 656 or in which the fulcrum component 634 is manufactured as a single component instead of the two component parts 634a, 634b as shown in the illustrated implementation.

The ramped fulcrum body 681 also has a small notch 691 formed therein, extending into the ramped fulcrum body 681 from the top ramping fulcrum surface 682 and near a location proximate to the ramping fulcrum surface's low-end position 683 (see FIGS. 6N2, 6N7, 6Q1, and 6Q2). In addition, the flexible circuit substrate structure portion 656a has a corresponding opening 677 (see FIGS. 6O1 and 6O3). The notch 691 and opening 677 allow a portion of the optical detector 660 to be positioned so that it extends through the opening 677 and into the notch 691 of the fulcrum body 681 where it is secured, as shown for example in FIG. 6N7.

The assembly process to create the assembled internal sensing system 637 (see FIG. 6J) may be accomplished as follows. First, the sub-assembly 608 shown in FIGS. 6O1-6O3 may be assembled with the first fulcrum component part 634a shown in FIGS. 6Q1-6Q3. In particular and referring to FIG. 6N2, the leg or extension portion 612 of the substrate structure 656 may be “wrapped around” the fulcrum body 681 and positioned so that the first, stationary portion 656a of the flexible circuit substrate 656 is supported thereunder by the rigid top ramping surface 682 and the second, flexing portion 656b of the flexible circuit substrate structure 656 extends beyond the inner side face 685 of the fulcrum body 681. This may be done by sliding the first substrate portion 656a into the horizontal opening 690 and flexing a proximal portion of the substrate 656 leg or extension 612 so that the main portion 614 is largely under the fulcrum component part 634a, except for a portion of which main substrate portion 614 that extends out from under the fulcrum component part 634a to the side of the fulcrum component part 634a, as shown in FIG. 6N1. With the sub-assembly 608 so assembled with the first fulcrum component part 634a, the optical detector 660 may be assembled therewith, positioning the detector 660 so that it extends through the opening 677 in the first substrate portion 656a and into the fulcrum body notch 691, so that the detector 660 becomes positioned as shown in FIG. 6N7. Next, the optical waveguide 654 may be positioned on the flexible circuit substrate 656, between the optical source 658 and the optical detector 660. While doing so, it may be desirable to provide temporary support under the second, flexing substrate portion 656b while the optical waveguide 658 is being placed on the substrate structure 656. Finally, the second fulcrum component part 634b may be attached to the first fulcrum component part 634a, bringing the two parts 634a, 634b together so that the extension 623 on the inner side of the second fulcrum component part 634b advances into the corresponding slot 620 on the inner side of the first fulcrum component part 634a. The two fulcrum component parts 634a, 634b may be secured together if needed by a snap-fit, gluing or some other connection mechanism. Alternatively, the two fulcrum component parts 634a, 634b may be secured together by virtue of constraints provided by the housing components 631, 632 within which the assembled internal sensing component 637 becomes housed.

Further description of the electro-optical motion sensing system 635 will now be provided, with reference to FIGS. 6O1-6O3 as well as FIG. 6N2. The sensing system 635 comprises the flexible circuit substrate 656, as well as optical, electro-optical, and electronic components provided upon the flexible circuit substrate structure 656, utilizing both sides of the substrate structure 656. The optical and electro-optical components include, in this embodiment, the optical emitter such as a light-emitting diode (LED) 658, the optical waveguide 1054 which may be a specially designed optical fiber component as described previously herein and constructed to enable optical power modulation techniques, and the optical detector 660. Various electronic components are provided on the flexible circuit substrate 656, mainly in this embodiment on the main portion 614 of the substrate structure 656 with mounting on both sides of the substrate structure 656 as shown. In the example of the FIG. 6 embodiment, the various electronic components include signal conditioning circuitry 627 that captures the analog signal output by the optical detector 660 and conditions the signal for further processing; a mixed signal microcontroller unit or “MPU” to perform various processing functions on the conditioned signal produced by the conditioning circuitry 627 (for example, the functions of MPU 462 described above in connection with FIG. 4, as well as control functions of the electro-optical components 658 and 660); and a wireless communications component 630 such as a Bluetooth or Bluetooth low energy (“BT” or “BLE”) integrated circuit chip. The wireless communication component may be assembled so that a circuitry portion 630a of the component 630 resides on the substrate structure 656 whereas an antenna portion 630b extends to the side of the substrate structure 656 as shown, for example in FIGS. 6O1 and 6O3. Also mounted on the substrate structure 656 is on-off switch connecting structure 670, which is connected to the cylindrical on-off switch spacer 616 and in turn to the on-off button 605, as shown for example in the cross-sectional diagram of FIG. 6I5. It will be appreciated that although the FIG. 6 design includes many discrete components, functions may be combined into one or more application specific integrated circuit (ASIC) components to achieve miniaturization and efficiencies in manufacturing.

The charging port 611 may be assembled with the main flexible circuit substrate portion 614 as shown in FIG. 6N1. In particular, two leads of the charging port 611 may be positioned in corresponding through-holes 669 provided near the side of the main flexible circuit substrate portion 614 as shown in FIG. 6N1. Additionally, the two leads 639 of the battery 628 (see FIG. 6J) may also be connected with the through-holes 669, so that electrical connection is made with the charging port 611 to recharge the battery 628 and also to provide the necessary electrical connection for the battery 628 to power the electro-optical motion sensing system 635. In the final assembly, the battery 628 resides above (above, in the orientation of FIG. 6N1) the main substrate structure portion 614, albeit spaced apart from the electronic components mounted thereon, as shown in FIG. 6I5 (which figure has a top-bottom orientation opposite that of FIG. 6N1. As shown in FIG. 6I5, the second dividing wall portion 626b and an inner retaining wall 678 formed in the top housing component 632 (see also FIG. 6K7) provide support for such spacing apart of the battery 628 from the main substrate portion 614 and mounted electronics.

The flexible circuit substrate 656 in this embodiment comprises the leg or extension portion 612 (which includes the first, stationary portion 656a and the second, flexing portion 656b), and the main portion 614. The main flexible circuit substrate portion 614 remains stationary during operation of the device 602 and includes the various electronic components mounted thereon (including for example, the conditioning circuitry 627, the MCU 629, and the wireless communication component 630). Interconnecting wires extend as needed within the main and extension portions 614, 612 of the substrate structure 656 to make electrical connections between the various electrical and electro-optical components, as one of skill in the art would understand. The first, stationary flexible circuit substrate portion 656a also remains stationary, in that when assembled as previously described the first substrate portion 656a is supported thereunder by the rigid top ramping fulcrum surface 682 of the fulcrum component 634. The first, stationary substrate portion 656a has mounted therewith the optical detector 660 and the first lengthwise portion 654a of the optical waveguide 654 (roughly, one half of the length of the optical waveguide 654). As such, first waveguide portion 654a thus remains stationary during operation.

The second flexible circuit substrate portion 656b, referred to herein as the flexing portion, has mounted therewith the optical emitter 658 and the remaining lengthwise portion (roughly one-half) 654b of the optical waveguide 654. The second, flexing substrate portion 656b may be positioned within a chamber within the device housing 615 so that the second, flexing substrate portion 656b has sufficient open space beneath to allow the second, flexing substrate portion 656b to flex downward in response to an external force applied from above during OPM operation. A supporting leaf spring 697 may be provided under a part of the first, stationary substrate portion 656a and extending to, and under, a part of the second, flexing substrate portion 656b, as illustrated in FIGS. 6N7, 6O1, and 6O3. This supporting leaf spring 697 supports, from underneath, the substrate 656 and optical waveguide 654 provided thereon. The leaf spring 697 resides in part under first, stationary substrate portion 656a and in part under the second, flexing substrate portion 656b. The part of the leaf structure 697 supporting the first substrate portion 656a thus rests directly upon the fulcrum body surface 682. As configured, the leaf spring 697 provides a spring force that returns the second, flexing substrate portion 656b and optical waveguide portion 654b provided thereon to an original resting or less flexed position when a force causing the flexing is removed or reduced.

In further detail during operation, the inner surface 651 of the button or pad structure 650, in response to forces applied against the skin facing surface 622 of the button or pad structure 650 resulting from the presence of arterial or other forces in the underlying blood vessel, will bear against a side of the second optical waveguide portion 654b and/or against the second, flexing substrate portion 656b upon which the optical waveguide portion 654b is positioned. The force applied against the waveguide portion 654b and/or the second, flexing substrate portion 656b causes the second, flexing substrate portion 656b as well as the second waveguide portion 654b supported thereon to flex downward and/or the second waveguide portion 654b to be compressed. As such, the optical output of the waveguide 654 (as determined by detector 660) may be modulated in accordance with the principles of optical power modulation described above.

As described previously in this document for example in connection with FIGS. 3A and 3B, modulation of the optical power output may be accomplished through flexing of the optical waveguide 654, compression of the optical waveguide 654 (which may in some embodiments be accomplished without the need for flexing of the second substrate portion 656b and optical waveguide portion 654b carried thereon), or a combination of flexing and compression. In a case where optical power modulation is accomplished through compression, it may be advantageous as described previously in this document that the flexible circuit substrate structure 656 generally be non-compressible as compared to the compressibility of the optical waveguide 654, such that a force applied against a side surface of the optical waveguide 654 results in compression of the waveguide 654 structure and not the underlying substrate 656.

The skin interfacing system 636 as previously described has a generally cylindrical button or pad structure 650 that extends through an opening 655 in the bottom housing component 631 so that a skin contacting surface 622 of the button or pad structure 650 is held, when in use, against a subject's skin adjacent an underlying blood vessel. The skin contacting surface 622 in this embodiment is generally flat in shape, although angled slightly to one side which may provide in some examples a better interface with the skin surface adjacent an underlying vessel. The interfacing component 636 also has, opposite the skin contacting surface 622, an inner surface 651 that bears against the optical waveguide portion 654b and/or flexible circuit substrate portion 656b of the electro-optical motion sensing system 635.

The leaf spring 652—whose structure and positioning has previously been described—is designed and configured to allow the button or pad structure 650 to flex downward upon added force being applied to the button or pad structures skin facing surface 622, and also cause the button or pad structure 650 to return to a resting state (that is, the button or pad structure 650 flexing back toward the skin surface) when the force applied against the button or pad structure skin facing surface 622 becomes reduced.

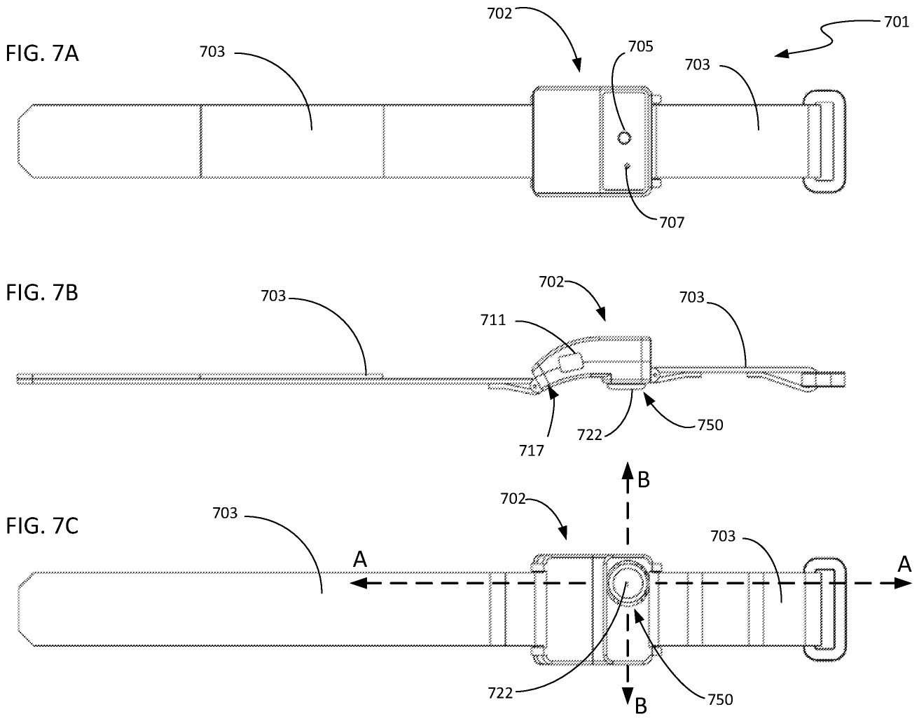

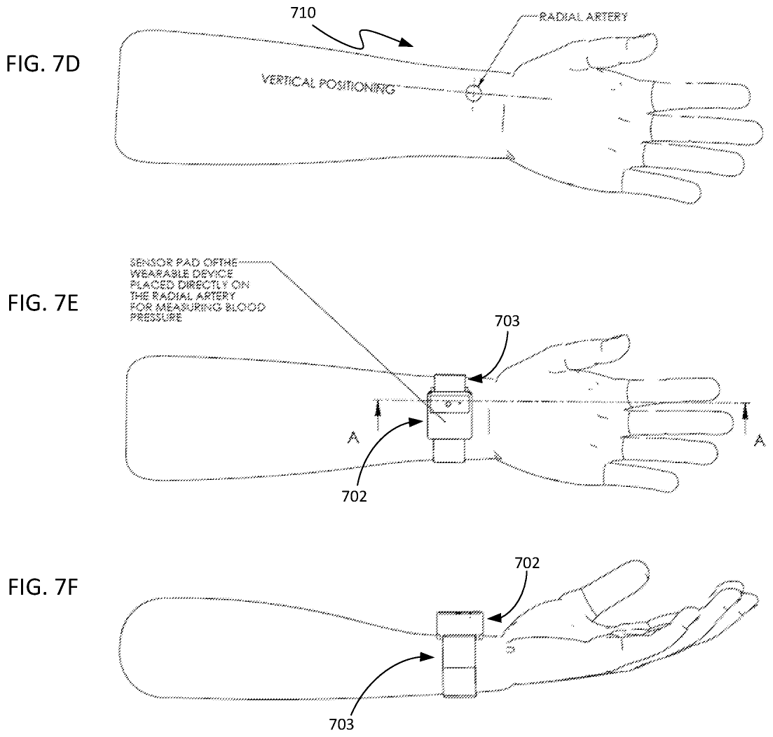

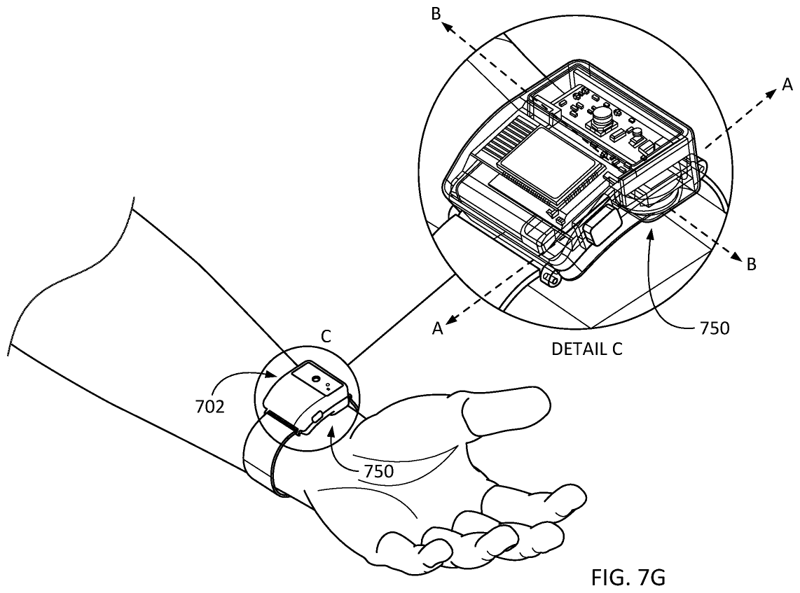

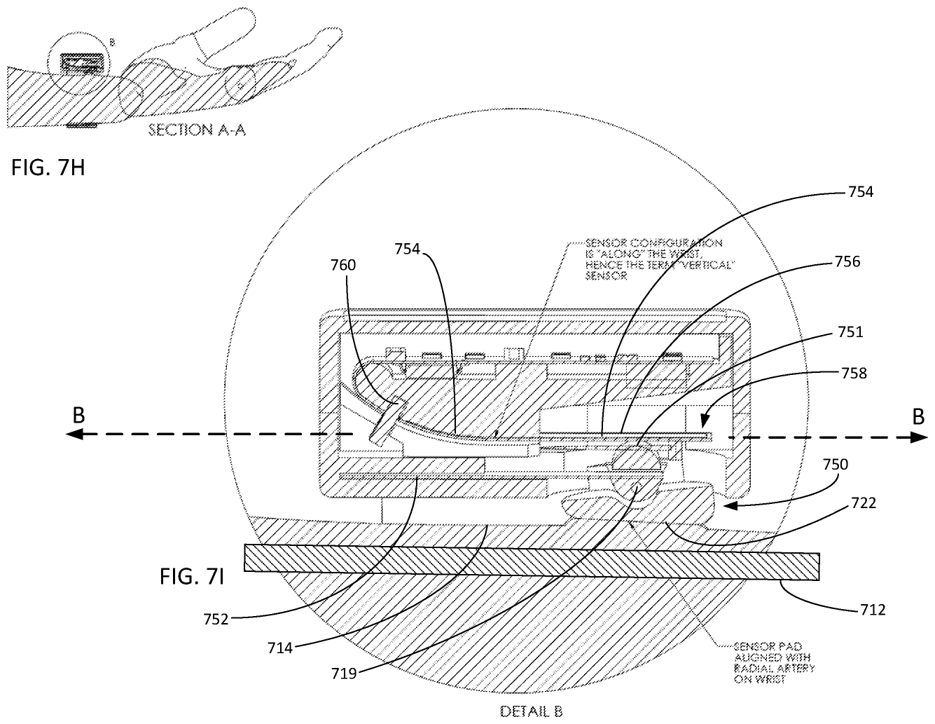

Another embodiment of a wrist-worn device 702 and band 703 similar in design to device 602 is shown in FIGS. 7A-7I. The monitoring device 702 is a wrist-worn device and monitors pressure in the radial artery, which is an artery that traverses the wrist. The monitoring device 702 is applied against the skin adjacent the radial artery on the underside of the wrist with the aid of a strap 703 that is applied around the wrist. As shown in FIG. 7A, the device 702 includes a press button 705 on a top side of a device housing, which press button 705 can be pressed by a user to turn the device 702 “on” and “off.” An indicator light 707 also situated on the top of the device housing lights up to indicate the device is “on,” and when not lighted indicates the device 702 is “off.” In this implementation, as with the implementation in FIGS. 6A-H, the housing configuration is such that it is intended to be worn on the left hand, given the positioning of a button 750 that needs to be positioned against the skin adjacent the radial artery.

Referring now to FIG. 7B, there is provided a side view of the device 702 and strap 703, with the straps 703 to each side of the device 702 to illustrate the contact points of the device 702 against a subject's wrist. A skin-contacting surface 722 of the button or pad structure 750 contacts the skin surface adjacent the radial artery. The housing of the device 702 also includes a bottom bearing surface 717 on a portion of the bottom surface of the housing that is opposite of where the button or pad structure 750 is positioned. Generally, there are two portions of the device 702 that contact the wrist when the device 702 is properly adjusted, with the strap 703 applying a proper amount of hold-down force, in a range for example of 5-15 mm Hg. Those two portions of the device 702 in contact with the wrist are the skin-contacting surface 722 of the button or pad structure 750 and the housing bottom bearing surface 717.

Referring to FIG. 7C, undersides of the device 702 and band 703 are shown, showing specifically the button or pad structure 750 on the underside of the device 702, as well as the skin-contacting surface 722 of the button. Labeled in FIG. 7C are two axes. The first is an axis labeled B-B, which shows the axis along which the optical waveguide of the sensing system extends, similar in design to the device 602 and sensor orientation shown in FIG. 6H. Referring again to FIG. 7C, the second axis labeled A-A illustrates an axis about which the button or pad structure 750 pivots, similar again to the structure of the device 602 as illustrated in FIG. 6H.

FIGS. 7D-7F show the positioning of the device 702 and band 703 on a wrist 710 of a subject, to measure blood pressure of the radial artery. The device 702 is positioned so that it is placed against the underside of the wrist, so that the button or pad structure 750 of the device 702 is placed directly on the skin adjacent the radial artery. In FIG. 7G, it is illustrated that the button or pad structure 750 is connected to the device 702 in a pivotable way that allows the button 750 to make better contact with the skin surface adjacent the artery. The pivoting occurs about an axis labeled A-A in FIG. 7G. FIG. 7G along with FIGS. 7H-I also illustrate the orientation of the optical waveguide 754 in the device 702. Specifically, the optical waveguide 754 extends generally along an axis labeled B-B, with an optical source 758 provided at one end of the waveguide 754, and an optical detector 760 provided at an opposite end of the waveguide 754. As such, the sensor may be considered to be oriented such that it extends “along” the wrist and “along” the radial artery and may be referred to as a “vertical” sensor.

Referring still to FIGS. 7H-I, there is provided a cross-section of the device 702 to illustrate its internal configuration. As was also shown in FIG. 7G, it is shown in FIGS. 7H-I that the optical waveguide 754 extends generally along an axis labeled B-B, with an optical source 758 provided on one end of the optical waveguide 754 and an optical detector 760 provided on an opposite end of the waveguide 754. The optical waveguide 754 is provided on a flexible and incompressible substrate 756, with the substrate 756 being illustrated in FIG. 7I as being above the waveguide 754. The right-half portion of the waveguide 754 and substrate 756 is allowed to flex upward by force of the button or pad structure 750. The left-half portion of the waveguide 754 and 756 has located directly above it a solid fulcrum structure, and therefore is prevented from flexing upward. The button or pad structure 750 in this embodiment is a two-part structure, with an upper half connected to an end of a leaf spring 752 and also pivotably connected with a pin or hinge structure 719 to a bottom half of the button or pad structure 750. The bottom half of the button or pad structure 750 has the outer skin-contacting surface 722, which is applied against the surface of the skin adjacent the radial artery 712. Here again it is shown that the optical waveguide is oriented such that it extends “along” the wrist and “along” the radial artery, and as such may be referred to as a “vertical” sensor.

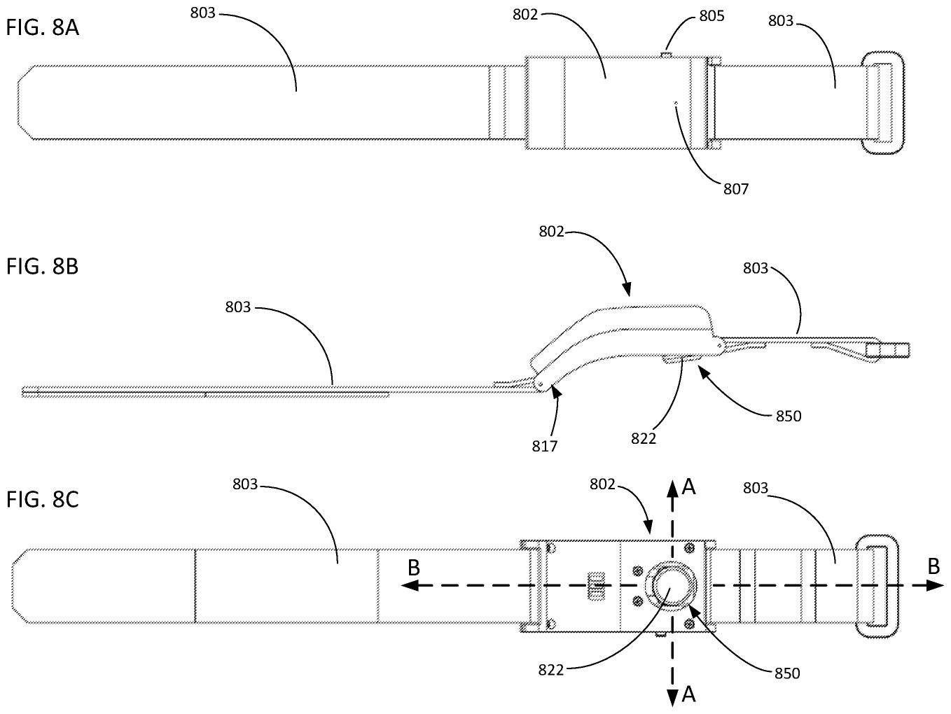

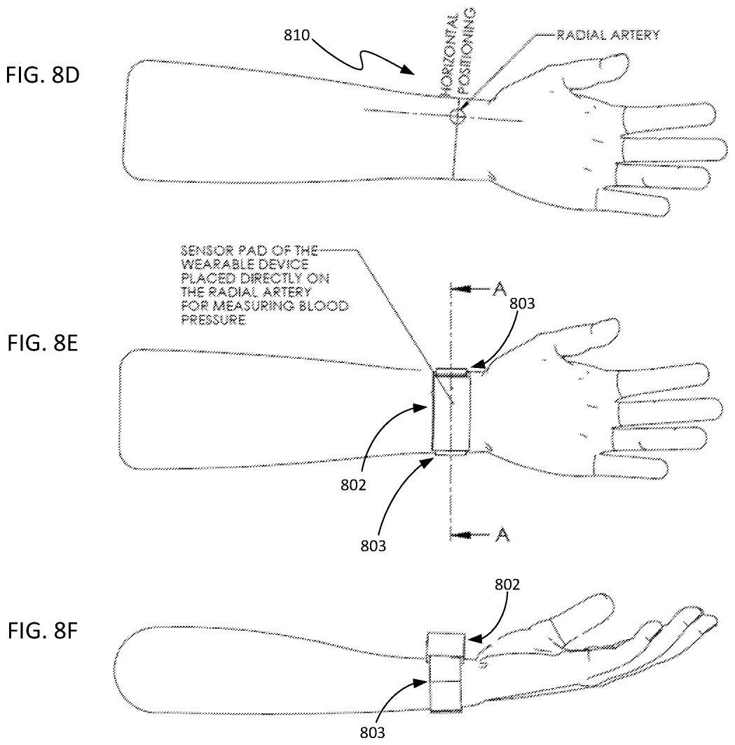

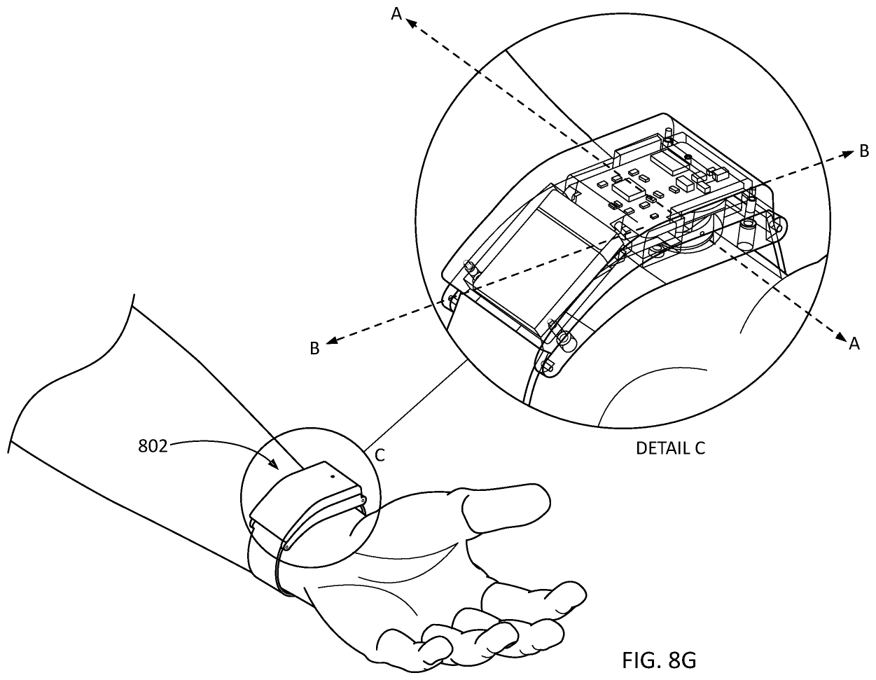

Another embodiment of a wrist-worn device 802 and band 803 is shown in FIGS. 8A-8I. The monitoring device 802 is a wrist-worn device and monitors pressure in the radial artery, which is an artery that traverses the wrist. The monitoring device 802 is applied against the skin adjacent the radial artery on the underside of the wrist with the aid of a strap 803 that is applied around the wrist. As shown in FIG. 8A, the device 802 includes a press button 805 on a side surface of a device housing, which press button 805 can be pressed by a user to turn the device 802 “on” and “off.” An indicator light 807 situated on a top surface of the device housing lights up to indicate the device is “on,” and when not lighted indicates the device 802 is “off.” In this implementation, the housing configuration is such that it may be worn on either the left or the right hand, given the positioning of a button 850 and the orientation of the optical sensing system, as will be illustrated in additional figures discussed below.

Referring now to FIG. 8B, there is provided a side view of the device 802 and strap 803 with the straps 803 to each side of the device 802, to illustrate the contact points of the device 802 against a subject's wrist. A skin-contacting surface 822 of the button or pad structure 850 contacts the skin surface adjacent the radial artery. The housing of the device 802 also includes a bottom bearing surface 817 on a portion of the bottom surface of housing that is opposite of where the button or pad structure 850 is positioned. Generally, there are two portions of the device 802 that contact the wrist when the device 802 is properly adjusted, with the strap 803 applying a proper amount of hold-down force, in a range for example of 5-15 mm Hg. Those two portions of the device 802 in contact with the wrist are the skin-contacting surface 822 of the button or pad structure 850 and the housing bottom bearing surface 817.

Referring to FIG. 8C, undersides of the device 802 and band 803 are shown, showing specifically the button or pad structure 850 on the underside of the device 802, as well as the skin-contacting surface 822 of the button. Labeled in FIG. 8C are two axes. The first is an axis labeled B-B, which shows the axis along which the optical waveguide of the sensing system extends, which is perpendicular to that in the devices 602 and 702 and sensor orientations shown in FIGS. 6H and 7G. Referring again to FIG. 8C, the second axis labeled A-A illustrates an axis about which the button or pad structure 850 pivots, again perpendicular to the structure of the devices 602 and 702 as illustrated in FIGS. 6H and 7G.

FIGS. 8D-8F show the positioning of the device 802 and band 803 on a wrist 810 of a subject, to measure blood pressure of the radial artery. The device 802 is positioned so that it is placed against the underside of the wrist, so that the button or pad structure 850 of the device 802 is placed directly on the skin adjacent the radial artery. In FIG. 8G, it is illustrated that the button or pad structure 850 is connected to the device 802 in a pivotable way that allows the button 850 to make better contact with the skin surface adjacent the artery. The pivoting occurs about an axis labeled A-A in FIG. 8G. FIG. 8G along with FIGS. 8H-I also illustrate the orientation of the optical waveguide 854 in the device 802, which is perpendicular to the orientation in devices 602 and 702. Specifically, the optical waveguide 854 extends generally along an axis labeled B-B, with an optical source 858 provided at one end of the waveguide 854, and an optical detector 860 provided at an opposite end of the waveguide 854. As such, the sensor may be considered to be oriented such that it extends “across” the wrist and “across” the radial artery and may be referred to as a “horizontal” sensor.

Referring still to FIGS. 8H-I, there is provided a cross-section of the device 802 to illustrate its internal configuration. As was also shown in FIG. 8G, it is shown in FIGS. 8H-I that the optical waveguide 754 extends generally along an axis labeled B-B, with an optical source 858 provided on one end of the optical waveguide 854 and an optical detector 860 provided on an opposite end of the waveguide 854. The optical waveguide 854 is provided on a flexible and incompressible substrate 856, with the substrate 856 being illustrated in FIG. 8I as being above the waveguide 854. The right-half portion of the waveguide 854 and substrate 856 is allowed to flex upward by force of the button or pad structure 850. The left-half portion of the waveguide 854 and substrate 856 has located directly above it a solid fulcrum structure, and therefore is prevented from flexing upward. The button or pad structure 850 in this embodiment is, as was the case in device 702, a two-part structure, with an upper half connected to an end of a leaf spring 852 and also pivotably connected with a pin or hinge structure 819 to a bottom half of the button or pad structure 850. The bottom half of the button or pad structure 850 has the outer skin-contacting surface 822, which is applied against the surface of the skin adjacent the radial artery 812. Here again it is shown that the optical waveguide is oriented such that it extends “across” the wrist and “perpendicular to” the radial artery, and as such may be referred to as a “horizontal” sensor.

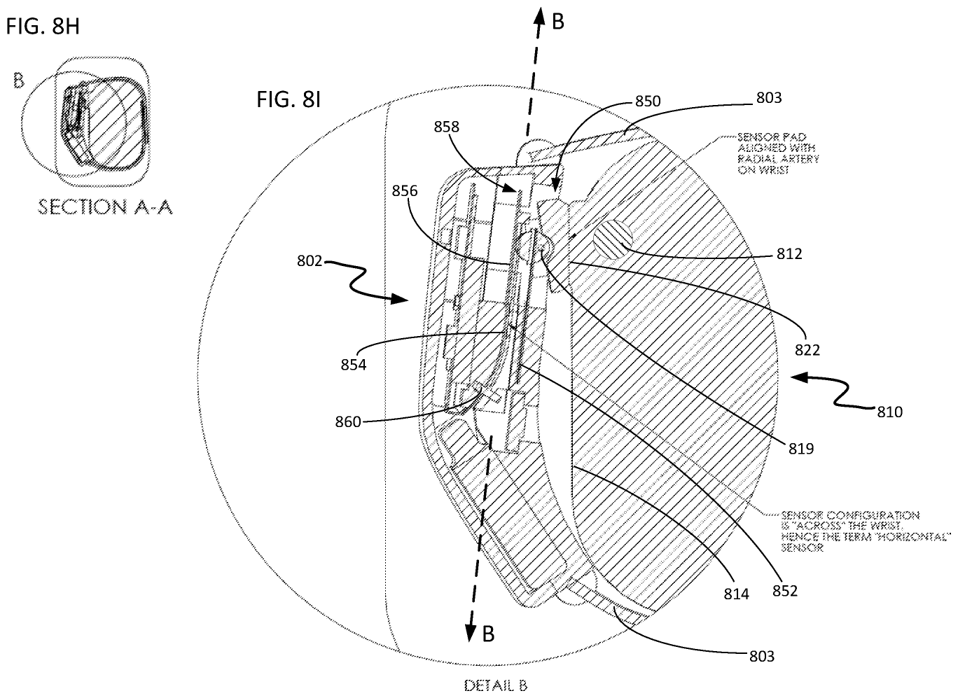

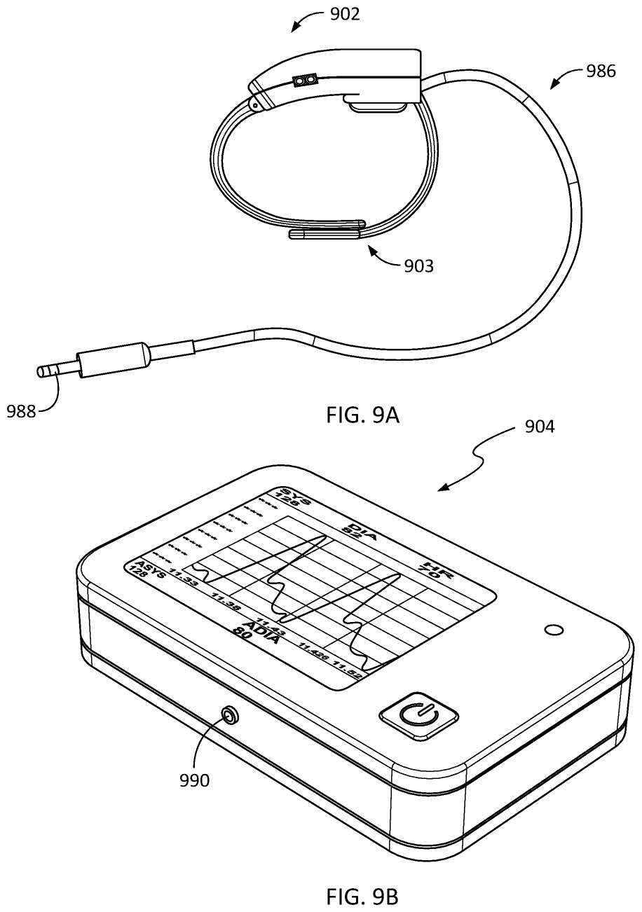

FIGS. 9A and 9B show another embodiment of a wrist-worn blood pressure monitoring device 902 and band 903, in combination with a dedicated monitor device with display. In this embodiment, unlike the wireless devices 602, 702 and 802 previously described, the device 902 is connectable to the dedicated monitor device 904 by a hard-wire connection. A wire connector structure 986 is provided with two male connector ends. One end is shown connected to a mating female connector provided in the device 902. At the other end of the connector structure 986 is another male connector 988, which may be plugged into a corresponding female connecting structure in the dedicated monitor 904.

In various embodiments of wrist-worn monitoring devices having micro-motion sensing structures and beat-to-beat blood pressure monitoring capability as previously described, the wrist-wearable device may take on various configurations. For example, the wrist-worn monitor device may include a watch face structure and a band structure, with the monitoring device and its associated button or pad structure being incorporated into the band structure. As such, on the top side of the wrist, a watch face may be provided, whereas the band may include the monitoring structures that are applied directly to the skin surface adjacent the radial artery on the bottom side of the wrist. In another embodiment, a self-contained sensor component may be incorporated within an inner chamber of a band structure. The sensor component may in this example provide an output of continuous blood pressure measurements that may be stored in memory for later download and/or may be provided for display on the watch face structure.

In another embodiment, a smart watch product embodiment may include a watch face structure and a band structure. Here again, a sensor device may be incorporated into the band structure. In one embodiment, a self-contained sensor component may be incorporated within an inner chamber of the band structure. The sensor component may, in this example, provide an output of continuous blood pressure measurements that may be stored in memory for later download and/or may be provided for display on the watch face structure.

In yet another embodiment, there is provided a stand-alone blood pressure monitoring wrist-worn product embodiment, which includes a clasp structure located such that it would be located on the top of the wrist, and a band structure. As with the two embodiments just described, a micro-motion sensor device may be incorporated into the band structure, such that it would be located on an inside surface of the band structure so that a button or pad structure may be placed against a surface of the skin adjacent the radial artery. In one embodiment, a self-contained sensor component may be incorporated within an inner chamber of the band structure. The micro-motion sensor component may, in this example, provide an output of continuous blood pressure measurements that may be stored in memory for later download and/or streamed by a wired or wireless connection for “real-time” display of the continuous blood pressure signal.

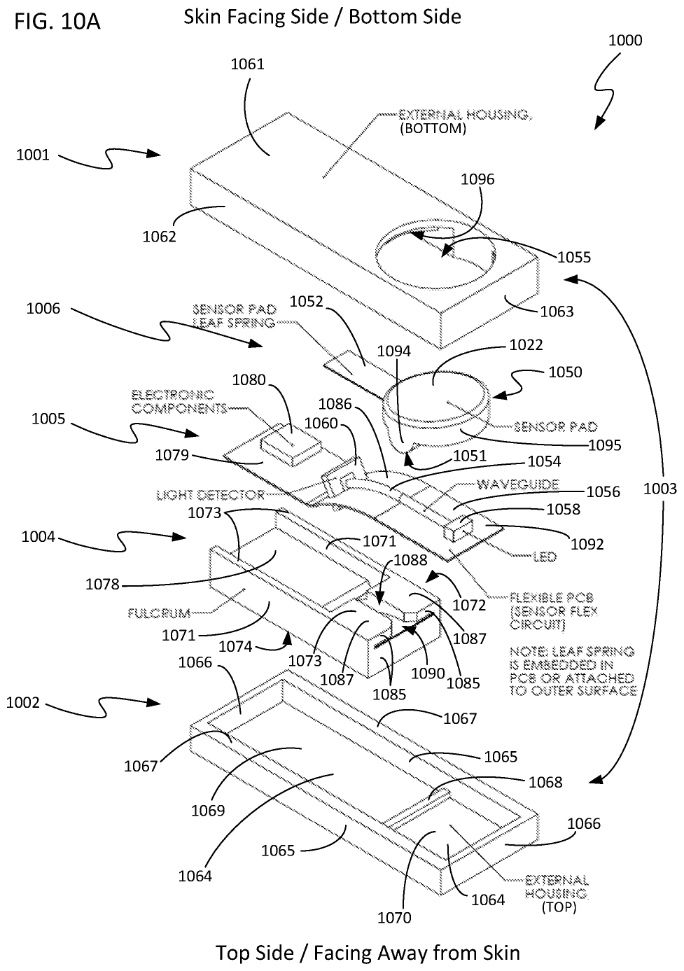

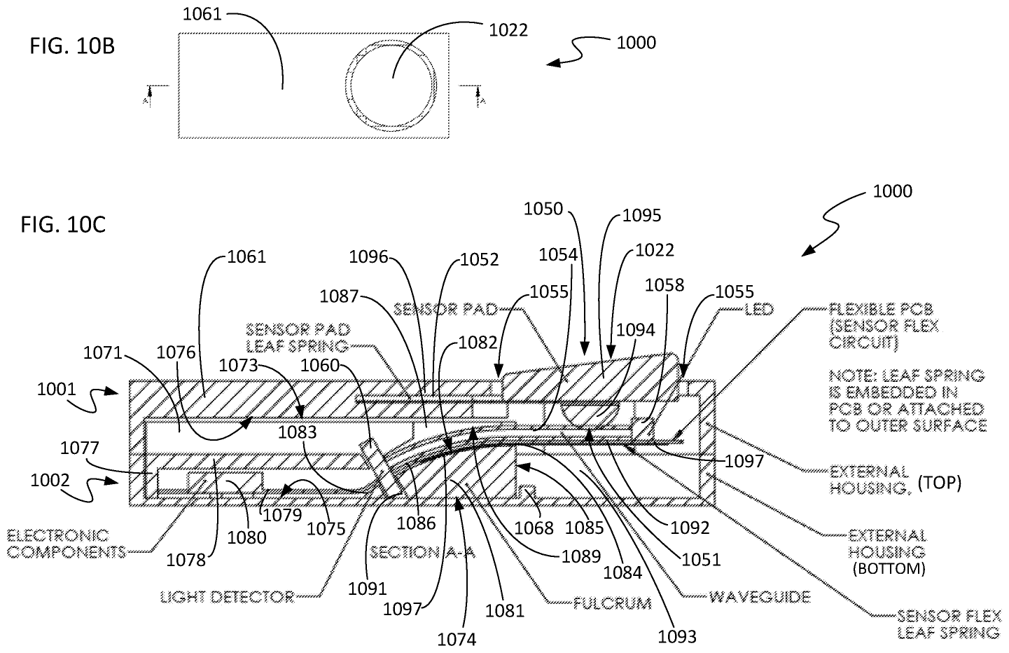

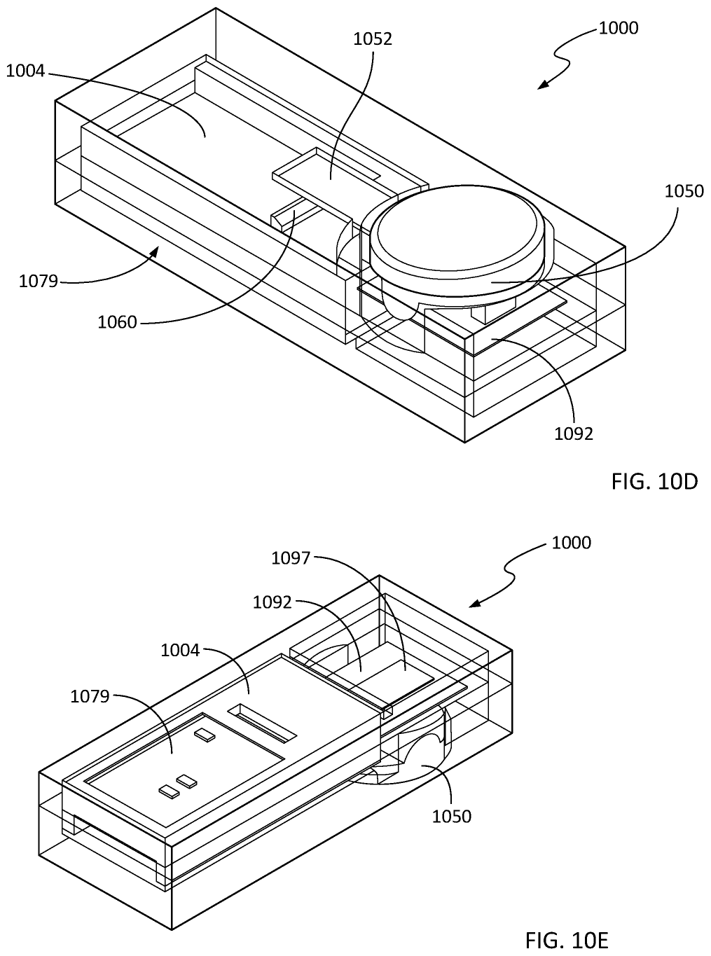

Turning now to FIGS. 10A-E, there is shown an additional design of a micro-motion sensing system 1000 adapted for use in non-invasive blood pressure monitoring devices, systems and methods, for example, in the devices and systems of FIGS. 1, 4, and 6-9. The non-invasive blood pressure monitoring systems and methods—in which the micro-motion sensor 1000 may be utilized—may provide continuous, “beat-to-beat” measures of blood pressure without the need for an inflatable cuff and without the need for calibration of the systems or methods for a particular subject using a separate blood pressure measurement system. In the example shown in FIGS. 10A-E, the micro-motion sensing system 1000 utilizes optical power modulation techniques for micro-motion sensing, as described above in connection with FIGS. 2, 3A-B, and 4. The micro-motion sensing system 1000 of FIGS. 10A-E may be utilized in a blood pressure monitoring device adapted to be worn or applied to a skin surface of a subject, adjacent an underlying blood vessel, to obtain a continuous blood pressure measurement.

In some configurations, the design of sensing system 1000 may provide a lower profile device size, as compared, for example, to implementations of certain micro-motion sensing system designs described above and shown in FIGS. 6, 7, and 8. In the FIGS. 6, 7, and 8 designs, a flexible circuit substrate component 656, 756, 856 is provided such that it may be said to be “wrapped” generally into a U-configuration, with the two legs of the “U” effectively being “stacked” on top of one another in relation to the surface of a subject's skin (see FIGS. 6N7, 7I, and 8I). In the micro-motion sensing system 1000 of FIGS. 10A-E, by contrast, a flexible circuit substrate structure 1056 is, instead, generally “flattened out” in what may be said to be a “flattened Z” configuration. In the illustrated “flattened Z” configuration, a flexing portion 1092 of the substrate structure 1056 carrying a lengthwise portion of an optical waveguide 1054 (roughly, one-half of the waveguide 1054, in this example) and an optical emitter 1058 is not provided in a “stacked” orientation with respect to any portion of a stationary portion 1079 of the substrate structure 1056 carrying electronic components 1080 such as processing circuitry, but instead are provided in two different lateral locations relative to the subject's skin when a device having the system 1000 is worn or applied against the skin as intended. As such, the micro-motion sensing system 1000 may enable a device into which the system 1000 is incorporated to have a lower, or more compact, profile relative to the surface of a subject's skin. Such a lower or more compact profile may be desirable in some cases in a wrist-wearable device, as an example.

In FIG. 10A, the micro-motion sensing system 1000 is shown in a perspective view and exploded to illustrate better its components. FIG. 10B is a bottom side (that is, skin-facing side) view of the system 1000. With regards to the orientation of “top” side and “bottom” side as used with respect to the housing components of the FIG. 10 embodiment, the “top” side is the side of the system that would be furthest from the skin and the “bottom” side is the side facing the skin, when a device incorporating the system is worn as intended. FIG. 10C is a longitudinal, vertical cross-sectional view along the plane A-A defined in FIG. 10B. FIGS. 10D and 10E are bottom and top isometric views, respectively, of the system 1000.

As illustrated in the exploded view of FIG. 10A, the micro-motion sensing system 1000 includes two external housing components—a bottom housing component 1001 and a top housing component 1002—adapted to be connected to one another to form an external system housing 1003 having a cuboid shape. Housing component 1001 is referred to herein as a “bottom” housing component because it is located nearest the skin when a device incorporating the system 1000 is worn, whereas housing component 1002 is referred to herein as a “top” housing component because it would be the outer surface furthest to the skin when a device incorporating the system 1000 is worn. A fulcrum component 1004 resides within an internal chamber formed by the two housing components 1001, 1002, when connected to one another to form the system external housing 1003. A “flattened Z” shaped electro-optical motion sensing system 1005 is, in part, carried by, and engaged against, the fulcrum component 1004. A skin interfacing system 1006—configured on one side to bear against a surface of a subject's skin when in use and on an opposite side bear against an optical waveguide 1054 and/or a flexible circuit substrate 1056 underlying the optical waveguide 1054, the substrate 1056 and optical waveguide 1054 being part of the electro-optical motion sensing system 1005—is fixed to the bottom housing component 1001 near an opening 1055 in the bottom housing component 1001.

In more detail, the bottom housing component 1001 has, in this embodiment, a cuboid shape. As such, the bottom housing component 1001 has a generally flat, rectangular bottom wall 1061; two generally flat, rectangular long side walls 1062 (the “long side” referring to a side of component 1001 that extends along the longest side dimension of the cuboid structure; only one of which long side walls 1062 being shown in FIG. 10A); two generally flat, rectangular short side walls 1063 (the “short side” referring to a side of component 1001 that extends along the shortest dimension of the cuboid structure; only one of which short side walls 1063 being shown in FIG. 10A). The side walls 1062, 1063 form a rectangular opening (not shown in FIG. 10A, in that it is on the underside of housing component 1001 as oriented in FIG. 10A) that is opposite the bottom wall 1061. A circular opening 1055 is provided in the bottom wall 1061, on one side of the bottom wall 1061, and positioned so that the generally cylindrically shaped button or pad structure 1050 of the skin interfacing component 1006 is aligned therewith so it is permitted to extend through the circular opening 1055 so that a skin contacting surface 1022 of the button or pad structure 1050 makes contact with, when in use as intended, the surface of the skin of a subject.

The top housing component 1002 has, in this embodiment, a cuboid shape with the same footprint as the bottom housing component 1001 to which the top housing component 1002 is mated to form the system external housing 1003. Top housing component 1002 has a generally flat, rectangular top wall 1064 of the same size as the rectangular bottom wall 1061; two generally flat, rectangular long side walls 1065 (the “long side” referring to a side of component 1002 that extends along the longest side dimension of the cuboid structure); two generally flat, rectangular short side walls 1066 (the “short side” referring to a side of component 1002 that extends along the shortest dimension of the cuboid structure); and a rectangular opening opposite the top wall 1064. Exposed bottom edges 1067 of the side walls 1065, 1066 of the top housing component 1002 are sized and configured to mate with exposed top edges (not shown in FIG. 10A) of the bottom side walls 1062, 1063 of the bottom housing component 1001. Connection of the bottom housing component 1001 to the top housing component 1002 may be provided by snap-fit mechanism, gluing, or any suitable fixation means. A straight dividing bar 1068 is provided on an inside surface of the top wall 1064, extending from an inside surface of one of the long side walls 1065 to an inside surface of the other of the two long side walls 1065. In this embodiment, the dividing bar 1068 is positioned such that it divides the top wall 1064 into two portions 1069, 1070, with portion 1069 covering roughly two-thirds of the top wall 1064 and portion 1070 covering roughly the remaining one-third of the top wall 1064.

The fulcrum component 1004 also has a generally cuboid shape, with a rectangular footprint sized so that the fulcrum component 1004 resides within a rectangular chamber defined by (and directly below) the rectangular portion 1069 of the top wall 1064. The fulcrum component 1004 has, on opposite sides, two generally flat, rectangular long side walls 1071. A fulcrum structure 1072 is provided at one of the long ends of the fulcrum component 1004, as shown, and is integral with the structure of the side walls 1071. The fulcrum component 1004 has a generally flat top surface 1073 including a top edge of the side walls 1071 and a top surface of the fulcrum structure 1072, and a generally flat bottom surface 1074 including a bottom edge of the side walls 1071 and a bottom surface of the fulcrum structure 1072. (Regarding “top” and “bottom” sides as they relate to the fulcrum component 1004, the “top” and “bottom” side are defined in the orientation of FIG. 10A, or in other words, the “top” side of the fulcrum component 1004 is the side closest to the skin surface when a device incorporating the system 1000 is worn as intended.) The height of the fulcrum component 1004 is sized such that—when the fulcrum component 1004 is assembled as intended within an internal chamber of the system external chamber 1003—the fulcrum component's generally flat bottom surface 1074 bears against or is very near an inside surface 1075 of the top housing component's top wall 1064, and such that the fulcrum component's generally flat bottom surface 1073 bears against or is very near an inside surface 1076 of the bottom housing component's bottom wall 1061. In other words, upon assembly the fulcrum component 1004 is “sandwiched” between the bottom and top housing components 1001, 1002. In addition, the dividing bar 1068 provided on the inside surface 1075 of the top housing component 1002 serves to prevent the sandwiched fulcrum component 1004 from sliding from a region within the inner chamber of the system housing 1003 directly below top wall portion 1069 and into the adjacent region of the module's inner chamber directly below top wall portion 1070. The height of the dividing bar 1068 may be shorter than the height of side walls 1065, 1066, as is the case in the FIG. 10A-E embodiment, although it will be appreciated that the height of dividing wall 1068 need only be configured to serve the function of containing the sandwiched fulcrum component 1004 so that it remains contained within a portion of the inner chamber of the system housing 1003 that is directly below (in other words, adjacent to) top wall portion 1069.

Further regarding the fulcrum component 1004, a short side wall 1077 (shown in FIG. 10C) is provided on one long end of the fulcrum component 1004, extending between the fulcrum component's two opposing long side walls 1071. The height of the short side wall 1077 is roughly one-half that of the long side walls 1071. A horizontal chamber dividing wall 1078 is provided that extends from a top edge of the short side wall 1077, perpendicular to the short side wall 1077, and specifically extends from the entire portion of the top edge of the short side wall 1077 between the inner surfaces of the fulcrum component's two opposing long side walls 1071. The horizontal chamber dividing wall 1078, in this embodiment, extends for a distance from the short side wall 1077 to slightly more than one-half the distance of the entire length of the fulcrum component 1004, as shown best in FIG. 10C. In the final assembly of the system 1000, a stationary portion 1079 of the motion sensing component's flexible circuit substrate 1056 (that is, the portion 1079 of the flexible circuit substrate 1056 having the electronic components 1080 provided thereon) is positioned on one side of (in other words, underneath) the fulcrum component's horizontal chamber dividing wall 1078, or more specifically as shown in FIG. 10C, is positioned in a region of the housing's inner chamber located under the horizontal dividing wall 1078 and above (“above,” in the orientation of FIG. 10C) the top housing component's top wall 1064.

The fulcrum structure 1072 includes a rounded-off ramped fulcrum body 1081, a top ramping fulcrum surface 1082 which (shown best in FIG. 10C) serves as a fulcrum for the optical waveguide 1054. As described previously, the optical waveguide 1054 is provided on the flexible substrate surface 1056, a portion of which optical waveguide 1054 flexes and/or compresses during operation of the system 1000 as described previously in accordance with techniques of optical power modulation. Specifically, the ramped fulcrum body 1081 has a generally flat bottom surface co-extensive with the entire flat bottom surface 1074 of the fulcrum component 1004. As such, the ramp structure's bottom surface 1074 engages, or is positioned near, the inner surface 1075 of the bottom housing component's bottom wall 1064. The ramp structure's top ramping fulcrum surface 1082 is opposite the generally flat bottom surface 1074, and extends between, and has a side-to-side orientation that is perpendicular to, the two fulcrum component side walls 1071; in other words, the side-to-side orientation of the top ramping fulcrum surface 1082 is generally parallel to the fulcrum component's bottom surface 1074. The top ramping fulcrum surface 1082—when the fulcrum component 1004 is assembled as intended with the top housing component 1002—rises or elevates from a low-end position 1083 that is adjacent to the top surface 1075 of the bottom housing component's bottom wall 1064. The top ramping fulcrum surface 1082 rises from low-end position 1083 and may be said to be “rounded off” in that its grade (steepness)—when viewing the top ramping fulcrum surface 1082 from left to right in the perspective of FIG. 10C, or in other words, when viewing from the low-end position 1083 of the top ramping fulcrum surface 1082 that is located near an end of the horizontal dividing wall 1078, to a high-end position 1084—declines as one moves up the ramping fulcrum surface 1082. The high-end position 1084 of the top ramping fulcrum surface 1082 is located at a side face 1085 of the fulcrum component 1004 (also the side face 1085 of the ramped fulcrum body 1081), which fulcrum component side face 1085 is positioned adjacent to, or nearly adjacent to, the bottom housing component's dividing bar 1068. Specifically, the shape of the top ramping fulcrum surface 1082 can be said to be “rounded off” in that the grade of the surface 1082 is initially steep (at about a 35-40 degree angle at the low-end position 1083) and then tapers such that the grade eventually becomes nearly horizontal at the high-end position 1084 of the top ramping fulcrum surface 1082. The high-end position 1084 of the top ramping fulcrum surface 1082 is located at an end portion 1084 of the ramped fulcrum body 1081, which is also the end portion 1084 of the entire fulcrum component 1073, as shown in FIG. 10C. In final assembly, a second portion 1086 of the motion sensing structure's flexible circuit substrate 1056, having an optical detector 1060 provided thereon along with a portion of the optical waveguide 1054, rests flush against the top ramping fulcrum surface 1082 (although with a leaf structure 1097 lying therebetween).

The fulcrum structure 1072 also includes two inwardly extending arms 1087, extending inwardly from, perpendicular to, and integral with the fulcrum component's two opposing long side walls 1071. A small vertical gap 1088 (shown in FIG. 10A) is provided between the two facing distal ends of the inwardly extending arms 1087. Top surfaces 1073 of the inwardly extending arms 1087 make up a portion of the generally flat top surface 1073 of the fulcrum component 1004, which top surface 1073 as described previously is positioned against or near the bottom housing component's bottom wall inside surface 1076 (noting that the bottom housing component is shown on top in FIG. 10C). The inwardly extending arms 1087 have ramping underside surfaces 1089 located opposite of the extending arm top surfaces 1073 and having a shape profile that is generally complementary to, and faces, the rounded-off ramp shape profile of the ramp structure's top ramping fulcrum surface 1082. A small horizontal gap or slot 1090 is provided between the inwardly extending arm underside surfaces 1089 and the ramp structure's top ramping fulcrum surface 1082. The small horizontal gap or slot 1090 provides a space for positioning, during assembly, the second portion 1086 of the flexible circuit substrate structure 1056, in a manner such that the substrate structure second portion 1086 is effectively sandwiched between the top ramping fulcrum surface 1082 and the inwardly extending arm underside surfaces 1089. The small vertical gap 1088 provided between the inwardly extending arms 1087 facilitates assembly wherein the optical waveguide 1054 may be advanced between the vertical gap 1088 and placed upon a surface of the flexible circuit substrate 1056 already positioned upon the fulcrum structure's top ramping fulcrum surface 1082. The ramped fulcrum body 1081 has a small notch 1091 formed therein, extending into the ramped fulcrum body 1081 from the top ramping fulcrum surface 1082 and near a location proximate to the ramping fulcrum surface's low-end position 1083. The notch 1091 allows a portion of the optical detector 1060 to be positioned and secured during assembly therein.

In some implementations, the fulcrum component 1004 may be provided in two parts, as with the fulcrum component 634 of the FIG. 6 embodiment having two parts 634a, 634b as shown in FIGS. 6P1-3 and 6Q1-3, with the inwardly extending arms being provided on separate parts of the fulcrum component 1004 and the fulcrum body 1081 being provided entirely with the first fulcrum part as with the FIG. 6 embodiment. In such a case, the electro-optical system 1005 may be assembled with a first fulcrum part by sliding the relevant portion of the system 1005 into the opening 1090 from the side so that the relevant portions of the electro-optical system are provided on top of the fulcrum body 1081. This assembly of the electro-optical system with the first fulcrum component part may be performed before a second fulcrum component part is assembled to the first fulcrum component part. In this case, the optical detector 1060 and optical waveguide may be assembled with the substrate structure 1056 before assembling the second fulcrum component part with the first fulcrum component part.

The electro-optical motion sensing system 1005 comprises a flexible circuit substrate 1056, as well as optical, electro-optical, and electrical components provided upon the flexible circuit substrate 1056. The optical and electro-optical components include, in this embodiment, an optical emitter such as a light-emitting diode (LED) 1058, an optical waveguide 1054 which may be a specially designed optical fiber component as described previously herein and constructed to enable optical power modulation techniques to be employed in the micro-motion sensing system 1000, and an optical detector 1060. Electronic components 1080 provided on the flexible circuit substrate 1056 may include functions of a microprocessor unit or “MPU” (such as the functions of MPU 462 described above in connection with FIG. 4), as well as control functions of the electro-optical components 1058, 1060.

The flexible circuit substrate 1056 in this embodiment may be defined as being made up of three portions 1079, 1086, 1092, specifically, a first, stationary flexible circuit substrate portion 1079; a second, stationary flexible circuit substrate portion 1086; and a third, flexing flexible circuit substrate portion 1092. The first flexible circuit substrate portion 1079 remains stationary during operation of the module. Electronic components 1080 are provided upon the first flexible circuit substrate portion 1079. Interconnecting wires extend as needed within all portions 1079, 1086, 1092 of the substrate structure 1056 to make electrical connections between the various electrical and electro-optical components, as one of skill in the art would understand. The second, stationary flexible circuit substrate portion 1086 also remains stationary in that it, when assembled as previously described, rests securely upon the top ramping fulcrum surface 1082 of the fulcrum component 1004. The second, stationary flexible circuit substrate portion 1086 carries the optical detector 1060 and a first portion of the optical waveguide 1054 (roughly, one half of the optical waveguide 1054) which portion of the optical waveguide 1054 thus remains stationary during operation. The third flexible circuit substrate portion 1092, referred to herein as a flexing portion 1092, carries the optical emitter 1058 and the remaining portion (roughly one-half) of the optical waveguide 1056. The third flexible circuit substrate portion 1092 may be positioned within the system housing 1003, as in the case of this embodiment, so that the third flexible circuit substrate portion 1092 has sufficient open space beneath, namely, open chamber 1093, allowing the third flexible circuit substrate portion 1092 to flex downward in response to an external force applied from above. (Here, “top” and “bottom, as well as for example “beneath” and “downward,” are defined with respect to the orientation of the fulcrum component 1004 and electro-optical system 1005 as shown in FIGS. 10A and 10C.) A supporting leaf spring 1097 may be provided under a part of the second, stationary flexible circuit substrate portion 1086 and extending to, and under, a part of the third, flexing flexible circuit substrate portion 1092, as illustrated in FIGS. 10C and 10E. This supporting leaf spring 1097 supports, from underneath, the substrate 1056 and optical waveguide 1054 provided thereon, and provides a spring force that returns the third, flexing substrate portion 1092 and optical waveguide 1054 back to an original resting or less flexed position when a force causing the flexing is removed or reduced.

In further detail during operation, an inner surface 1051 of the button or pad structure 1050, in response to forces applied against the skin facing surface 1022 of the button or pad structure 1050 resulting from the presence of arterial or other waves in the underlying blood vessel, will bear similar forces against a side of the optical waveguide 1054 and/or against the flexible circuit substrate 1056 upon which the optical waveguide 1054 is positioned. The force applied against the waveguide 1056 and/or third, flexing substrate portion 1092 causes the third, flexing substrate portion 1092, as well as the portion of the optical waveguide 1054 carried thereon, to flex downward. As such, the optical output of the waveguide 1054 may be modulated in accordance with the principles of optical power modulation describe above.

As described previously in this document, for example in connection with FIGS. 3A and 3B, modulation of the optical power output may be accomplished through flexing of the optical waveguide 1054, compression of the optical waveguide 1054 (which may in some embodiments be accomplished without the need for the third flexible circuit substrate portion 1092 and optical waveguide 1054 carried thereon to flex downward as described), or a combination of flexing and compression. In a case where optical power modulation is accomplished through compression, it may be advantageous as described previously in this document that the flexible circuit substrate structure 1056 generally be non-compressible as compared to the compressibility of the optical waveguide 1054, such that when a force applied against a side surface of the optical waveguide 1054 results in compression of the waveguide 1054 structure and not the underlying substrate 1056.

The skin interfacing system 1006 has a generally cylindrical button or pad structure 1050 that extends through an opening 1055 in the bottom housing component 1001 so that a skin contacting surface 1022 of the button or pad structure 1050 is held, when in use, against a subject's skin adjacent an underlying blood vessel. The skin contacting surface 1022 in this embodiment is generally flat in shape, although angled slightly to one side, which may provide in some examples a better interface with the skin surface adjacent an underlying vessel. The interfacing component 1006 also has, opposite the skin contacting surface 1022, an inner surface 1051 that bears against the optical waveguide 1054 and/or flexible circuit substrate 1056 of the electro-optical motion sensing component 1074. In this embodiment, the inner contacting surface 1051 is provided on an inner half-cylinder structure 1094 which is provided at, and integral with, an inner portion of an outer cylindrical portion 1095 of the button or pad structure 1050. The inner half-cylinder structure 1094 includes the inner contacting surface 1051 and is oriented such that its longitudinal axis is generally perpendicular to a longitudinal axis of the outer cylindrical portion 1095.

A leaf spring 1052 fixedly attaches at one end to a side of the outer cylindrical portion 1095 of the button or pad structure 1050, and at an opposite end is fixedly attached to the bottom housing component 1001. The leaf spring 1052 is designed and configured to allow the button or pad structure 1050 to flex downward (downward as defined in the orientation of FIG. 10C) upon added force being applied to the button or pad structures skin facing surface 1022, and also cause the button or pad structure 1050 to return to a resting state (that is, the button or pad structure 1050 flexing back upward) when the force applied against surface 1022 reduces. The fixation of the leaf spring 1052 to the bottom housing component 1001 may be provided by the leaf spring 1052 being fixedly secured into a horizontal channel 1096 formed in the bottom wall 1061, wherein the channel 1096 is formed in the side of a borehole of the bottom wall 1061 that provides for the cylindrical opening 1055.

Referring now to the bottom and top isometric views of FIGS. 10D and 10E, it can be seen that the flexing portion 1092 of the substrate component 1056 carrying a portion of an optical waveguide 1052 and an optical emitter 1058 is not provided in a “stacked” orientation with a stationary portion 1079 of the substrate component 1056 carrying electronic components 1080 such as processing circuitry, but instead are provided in two different lateral locations relative to the subject's skin when a device having the system 1000 is worn or applied against the skin as intended. As such, the micro-motion sensing system 1000 may enable a device into which the system 1000 is incorporated to have a lower, or more compact, profile relative to the surface of a subject's skin. Such a lower or more compact profile may be desirable in some cases in a wrist-wearable device, as an example.

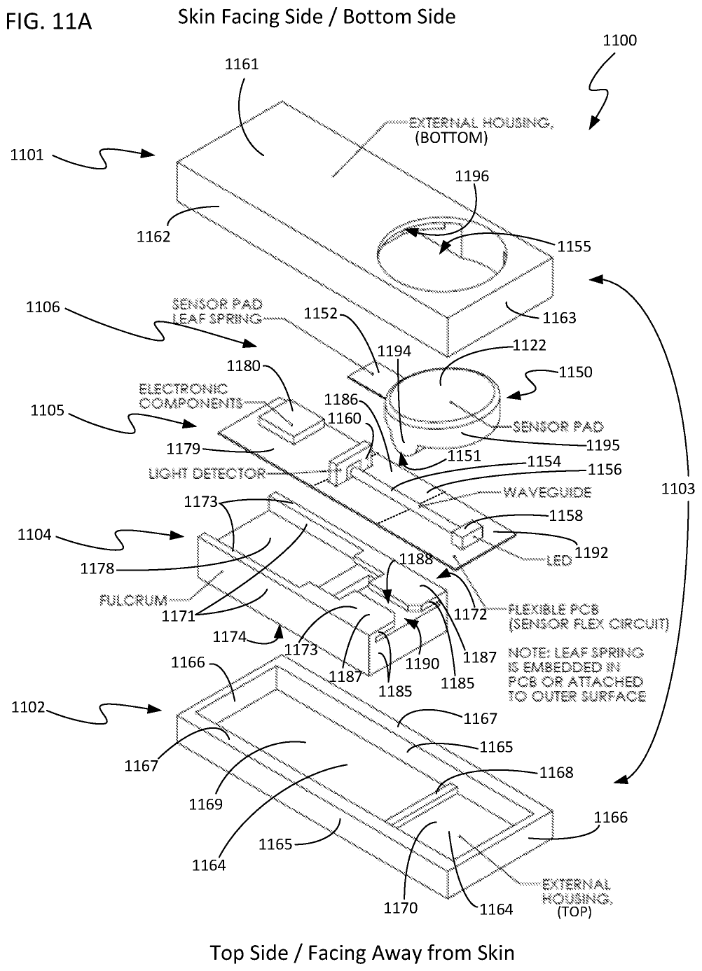

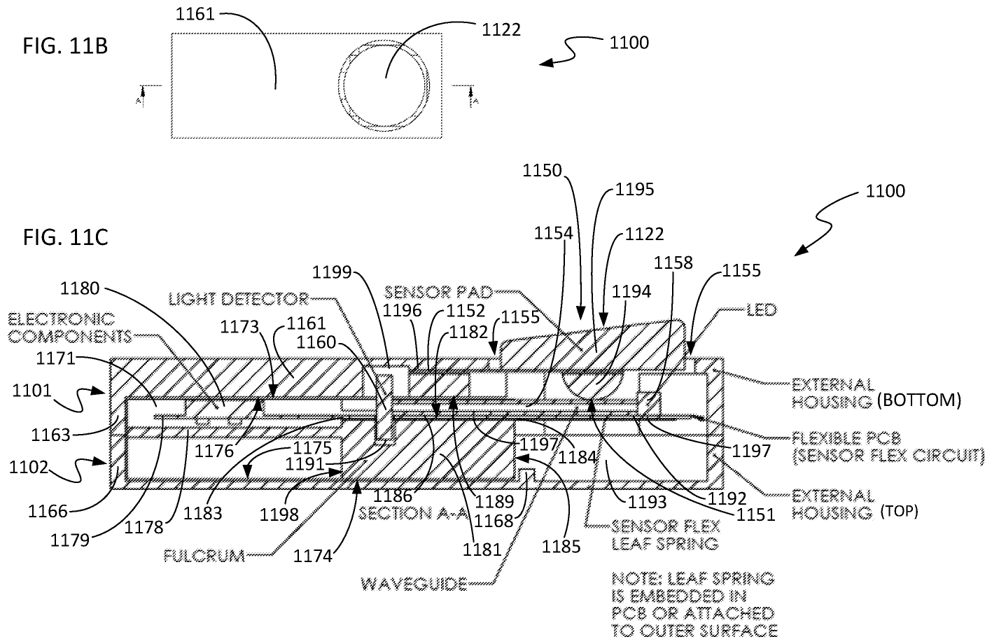

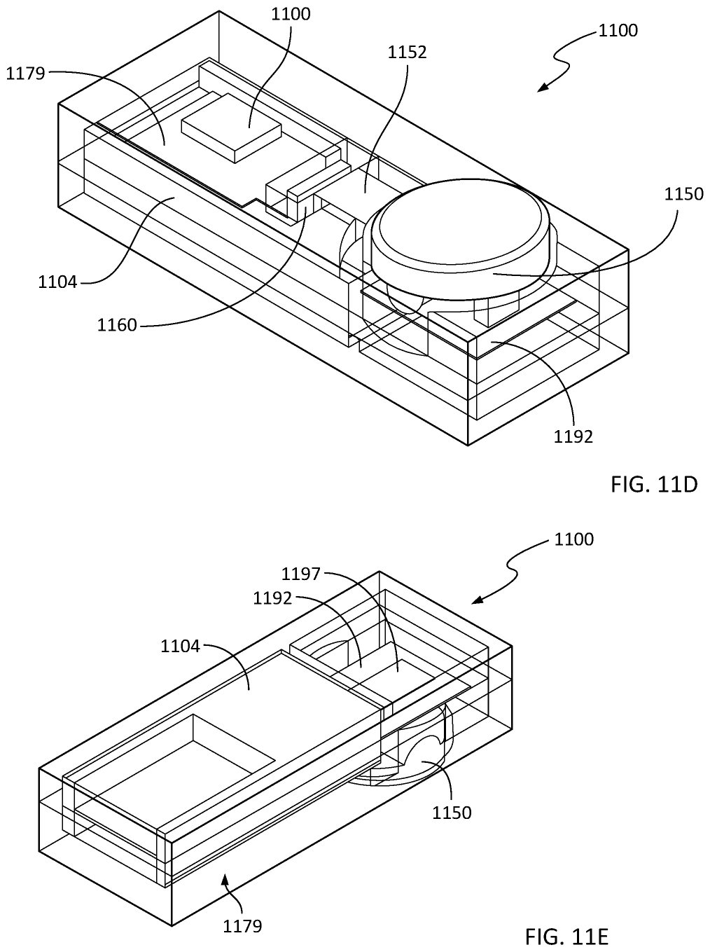

Turning now to FIGS. 11A-E, there is shown an additional design of a micro-motion sensing system 1100 adapted for use in non-invasive blood pressure monitoring devices, systems and methods, for example, as in the devices and systems of FIGS. 1, 4, and 6-9. The non-invasive blood pressure monitoring devices, systems and methods—in which the micro-motion sensing system 1100 may be utilized—may provide continuous, “beat-to-beat” measures of blood pressure without the need for an inflatable cuff and without the need for calibration of the systems or methods for a particular subject using a separate blood pressure measurement system. In the example shown in FIGS. 11A-E, the micro-motion sensing system 1100 utilizes optical power modulation techniques for micro-motion sensing, as described above in connection with FIGS. 2, 3A-B, and 4. The micro-motion sensing system 1100 of FIGS. 11A-E may be utilized in a blood pressure monitoring device adapted to be worn or applied to a skin surface of a subject, adjacent an underlying blood vessel, to obtain a continuous blood pressure measurement.

In some configurations, the design of sensing system 1100 of FIGS. 11A-E, may be similar to the design of the sensing system 1000 of FIGS. 10A-E, may provide a lower profile device size, as compared, for example, to implementations of certain micro-motion sensing system designs described above and shown in FIGS. 6, 7, and 8. In the FIGS. 6, 7, and 8 designs, a flexible circuit substrate component 656, 756, 856 is provided such that it may be said to be “wrapped” generally into a U-configuration, with the two legs of the “U” effectively being “stacked” on top of one another in relation to the surface of a subject's skin. In the micro-motion sensing system 1100 of FIGS. 11A-E, by contrast, a flexible circuit substrate component 1156 is, instead, generally “flattened out” in what may be said to be a “completely flattened” configuration. In the illustrated “completely flattened” configuration, a flexing portion 1192 of the substrate component 1156 carrying a portion of an optical waveguide 1154 and an optical emitter 1158 is not provided in a “stacked” orientation with respect to a stationary portion 1179 of the substrate component 1156 carrying electronic components 1180 such as processing circuitry, but instead are provided in two different lateral locations relative to the subject's skin when a device having the system 1100 is worn or applied against the skin as intended. As such, the micro-motion sensing system 1100 may enable a device into which the system 1100 is incorporated to have a lower, or more compact, profile relative to the surface of a subject's skin. Such a lower or more compact profile may be desirable in some cases in a wrist-wearable device, as an example.

In FIG. 11A, the micro-motion sensing system 1100 is shown in a perspective view and exploded to illustrate better its components. FIG. 1lB is a bottom side view of the system 1100. With regards to the orientation of “top” side and “bottom” side as used with respect to the housing components of the FIG. 11 embodiment, the “top” side is the side of the system that would be furthest from the skin and the “bottom” side is the side facing the skin, when a device incorporating the system is worn as intended. FIG. 11C is a longitudinal, vertical cross-sectional view along the plane A-A defined in FIG. 11B. FIGS. 11D and 11E are bottom and top isometric views, respectively, of the system 1100.

As illustrated in the exploded view of FIG. 11A, the micro-motion sensing system 1100 includes two external housing components—a bottom housing component 1101 and a top housing component 1102—adapted to be connected to one another as shown in FIGS. 10C-E to form an external module housing 1103 having a cuboid shape. Housing component 1001 is referred to herein as a “bottom” housing component because it is located nearest the skin when a device incorporating the system 1000 is worn, whereas housing component 1002 is referred to herein as a “top” housing component because it would be the outer surface furthest to the skin when a device incorporating the system 1000 is worn. A fulcrum component 1104 resides within an internal chamber formed by the two housing components 1102, 1103, when connected to one another to form the module external housing 1103. A “completely flattened” shaped electro-optical motion sensing component 1105 is, in part, carried by, and engaged against, the fulcrum component 1104. A skin interfacing system 1106—configured on one side to bear against a surface of a subject's skin when in use as intended, and on an opposite side bear against an optical waveguide 1154 and/or a flexible circuit substrate 1156 underlying the optical waveguide 1154, the substrate 1156 and optical waveguide 1154 being part of the electro-optical motion sensing component 1105—is fixed to the bottom housing component 1101 near an opening 1155 in the bottom housing component 1101.

In more detail, the bottom housing component 1101 has, in the FIG. 11A-E embodiment as in the FIG. 10A-E embodiment, a cuboid shape. As such, the bottom housing component 1101 has a generally flat, rectangular bottom wall 1161; two generally flat, rectangular long side walls 1162 (the “long side” referring to a side of component 1101 that extends along the longest side dimension of the cuboid structure; only one of which long side walls 1162 being shown in FIG. 11A); and two generally flat, rectangular short side walls 1163 (the “short side” referring to a side of component 1101 that extends along the shortest dimension of the cuboid structure; only one of which short side walls 1163 being shown in FIG. 11A). The side walls 1162, 1163 form a rectangular opening (not shown in FIG. 11A, in that it is on the underside of module 1101 as oriented in FIG. 11A) that is opposite the bottom wall 1161. A circular opening 1155 is provided in the bottom wall 1161, on one side of the bottom wall 1161, and positioned so that the generally cylindrically shaped button or pad structure 1150 of the skin interfacing component 1106 is aligned therewith so it is permitted to extend through the circular opening 1155 so that a skin contacting surface 1122 of the button or pad structure 1150 makes contact with, when in use as intended, the surface of the skin of a subject.

The top housing component 1102 has, in this embodiment as with the FIG. 10A-E embodiment, a cuboid shape with the same footprint as the bottom housing component 1101 to which the top housing component 1102 is mated and engaged to form the system external housing 1103. Top housing component 1102 has a generally flat, rectangular top wall 1164 of the same size as the rectangular bottom wall 1161; two generally flat, rectangular long side walls 1165 of the same length as long side walls 1062 of the bottom housing component 1101 (the “long side” referring to a side of component 1102 that extends along the longest side dimension of the cuboid structure); and two generally flat, rectangular short side walls 1166 of the same length as the short side walls 1163 of the bottom housing component 1101 (the “short side” referring to a side of component 1102 that extends along the shortest dimension of the cuboid structure); and a rectangular opening opposite the top wall 1164. Exposed edges 1167 of the side walls 1165, 1166 of the top housing component 1102 are sized and configured to mate with exposed edges (not shown in FIG. 11A) of the bottom side walls 1162, 1163 of the bottom housing component 1101. Connection of the bottom housing component 1101 to the top housing component 1102 may be provided by snap-fit mechanism, gluing, or any suitable fixation means. A straight dividing bar 1168 is provided on an inside surface of the top wall 1164, extending from an inner surface of one of the long side walls 1165 to an inner surface of the other of the two long side walls 1165. In this embodiment, the dividing bar 1168 is positioned such that it divides the top wall 1164 into two portions 1169, 1170, with portion 1169 covering roughly two-thirds of the top wall 1164 area and portion 1170 covering roughly the remaining one-third of the top wall 1164 area.

The fulcrum component 1104 also has a generally cuboid shape, with a rectangular footprint sized so that the fulcrum component 1104 resides within a rectangular chamber defined by (and directly below) the rectangular portion 1169 of the top wall 1164. The fulcrum component 1104 has, on opposite sides, two generally flat, rectangular long side walls 1171. A fulcrum structure 1172 is provided at one of the long ends of the fulcrum component 1104, as shown, and is integral with the structure of the side walls 1171. The fulcrum component 1104 has a generally flat top surface 1173 including a top edge of the side walls 1171 and a top surface of the fulcrum structure 1172, and a generally flat bottom surface 1174 including a bottom edge of the side walls 1171 and a bottom surface of the fulcrum structure 1172. (Regarding “top” and “bottom” sides as they relate to the fulcrum component 1004, the “top” and “bottom” side are defined in the orientation of FIGS. 10A and 10C, or in other words, the “top” side of the fulcrum component 1004 is the side closest to the skin surface when a device incorporating the system 1000 is worn as intended.) The height of the fulcrum component 1104 is sized such that—when the fulcrum component 1104 is assembled as intended within an internal chamber of the module external chamber 1103—the fulcrum component's generally flat bottom surface 1174 bears against, or is very near, an inside surface 1175 of the top housing component's top wall 1164, and the fulcrum component's generally flat top surface 1173 bears against, or is very near, an inside surface 1176 of the bottom housing component's bottom wall 1161. In other words, upon assembly the fulcrum component 1104 is “sandwiched” between the bottom and top housing components 1101, 1102. In addition, the dividing bar 1168 provided on the inside surface 1175 of the top wall 1164 of the top housing component 1102 serves to prevent the sandwiched fulcrum component 1104 from sliding from a region within the inner chamber of the housing 1103 directly below top wall portion 1169 and into the adjacent region of the system's inner chamber directly below top wall portion 1170. The height of the dividing bar 1168 may be shorter than the height of side walls 1165, 1166, as is the case in the FIG. 10A-E embodiment, although it will be appreciated that the height of the dividing wall 1168 need only be configured to serve the function of containing the sandwiched fulcrum component 1104 so that it remains contained within a portion of the inner chamber of the module housing 1103 that is directly below (in other words, adjacent to) the top wall portion 1169.

Further regarding the fulcrum component 1104, a generally planar horizontal chamber dividing wall 1178 is provided that extends from, and perpendicular to, an inner side face 1198 of the fulcrum body 1181. More specifically, with the fulcrum component assembled within housing components 1101, 1102, the horizontal dividing wall 1178 extends from the inner fulcrum body side face 1198 for the entire remaining length of the fulcrum component 1104, to abut against a portion of inner surfaces of the top and bottom housing component short side walls 1163, 1166, as shown best in FIG. 11C. The horizontal chamber dividing wall 1178, in this embodiment, extends for a distance of slightly more than one-half the entire length of the fulcrum component 1104, as shown best in FIG. 10C. The horizontal chamber dividing wall 1178 also extends entirely between the inner surfaces of the fulcrum component's two opposing long side walls 1171. In the final assembly of the system 1100, a stationary portion 1179 of the motion sensing component's flexible circuit substrate 1056 (that is, the portion 1179 of the flexible circuit substrate 1156 having the electronic components 1180 provided thereon) is positioned on one side of (in other words, above) the fulcrum component's horizontal chamber dividing wall 1178, or more specifically as shown in FIG. 10C, is positioned in a region of the housing's inner chamber located above the horizontal dividing wall 1178 and below the bottom housing component's bottom wall 1161 (here, “above” and “below” are defined in the orientation of FIG. 10C).

The fulcrum structure 1172 includes fulcrum body 1181, a top generally flat and horizontal fulcrum surface 1182 of which (shown best in FIG. 11C) serves as a fulcrum for the optical waveguide 1154. As described previously, the optical waveguide 1154 is provided on the flexible substrate surface 1156, a portion of which optical waveguide 1154 flexes and/or compresses during operation of the system 1100 as described previously in accordance with techniques of optical power modulation. Specifically, the fulcrum body 1181 has a generally flat and horizontal bottom surface co-extensive with the entire flat bottom surface 1174 of the fulcrum component 1104. As such, the fulcrum body's bottom surface 1174 engages, or is positioned near, the inside surface 1175 of the top housing component's top wall 1164. The fulcrum body's top fulcrum surface 1182 is opposite the generally flat bottom surface 1174, and extends between, and perpendicular to, the two fulcrum component side walls 1171; in other words, the top fulcrum surface 1182 lies in an orientation that is generally parallel to the fulcrum component's bottom surface 1174. The top fulcrum surface 1182 (identified in FIG. 11C)—when the fulcrum component 1104 is assembled as intended with the top housing component 1102—is a generally flat and horizontal surface, from a first end position 1183 of the top fulcrum surface 1182 that is located near a location from which the horizontal dividing wall 1178 starts to extend, to a second end position 1184 located at a side face 1185 of the fulcrum component 1104 (also the side face 1185 of the fulcrum body 1181). The fulcrum component side face 1185 is positioned adjacent to, or nearly adjacent to, the top housing component's dividing bar 1168. In final assembly, a second portion 1186 of the motion sensing structure's flexible circuit substrate 1156, having an optical detector 1160 provided thereon along with a portion of the optical waveguide 1154, rests flush against the top fulcrum surface 1182 (although with a leaf structure 1197 lying therebetween).

The fulcrum structure 1172 also includes two inwardly extending arms 1187, extending inwardly from, perpendicular to, and integral with the fulcrum component's two opposing long side walls 1171. A small vertical gap 1188 (shown in FIG. 11A) is provided between the two facing distal ends of the inwardly extending arms 1187. Top surfaces 1173 of the inwardly extending arms 1187 make up a portion of the generally flat top surface 1173 of the fulcrum component 1104, which top surface 1173 as described previously is positioned against or near the bottom housing component's bottom wall inside surface 1176 (noting that the bottom housing component is shown on top in FIG. 11C). The inwardly extending arms 1187 have underside surfaces 1189, located opposite of the inwardly extending arm top surfaces 1173 and having a flat and generally horizontal shape profile that is generally complementary to, and faces, the generally flat and horizontal shape profile of the fulcrum body's top fulcrum surface 1182. A small horizontal gap or slot 1190 is provided between the inwardly extending arm underside surfaces 1189 and the fulcrum body's top fulcrum surface 1182. The small horizontal gap or slot 1190 provides a space for positioning, during assembly, the second portion 1186 of the flexible circuit substrate structure 1156, in a manner such that the substrate structure second portion 1186 is effectively sandwiched in part between the top fulcrum surface 1182 and the extending arm underside surfaces 1189. The small vertical gap 1188 provided between the inwardly extending arms 1187 facilitates assembly wherein the optical waveguide 1154 may be advanced between the vertical gap 1188 and placed upon a surface of the flexible circuit substrate 1156 already positioned upon the fulcrum body's top fulcrum surface 1182. The fulcrum body 1181 has a small notch 1191 formed therein, extending into the fulcrum body 1181 from the top fulcrum surface 1182 and near a location proximate to the fulcrum surface's first end position 1183. The notch 1191 allows a portion of the optical detector 1160 to be positioned and secured during assembly therein.

In some implementations, the fulcrum component 1004 may be provided in two parts, as with the fulcrum component 634 of the FIG. 6 embodiment having two parts 634a, 634b as shown in FIGS. 6P1-3 and 6Q1-3, with the inwardly extending arms being provided on separate parts of the fulcrum component 1104 and the fulcrum body 1181 being provided entirely with the first fulcrum part as with the FIG. 6 embodiment. In such a case, the electro-optical system 1105 may be assembled with a first fulcrum part by sliding the relevant portion of the system 1105 into the opening 1190 from the side so that the relevant portions of the electro-optical system are provided on top of the fulcrum body 1181. This assembly of the electro-optical system with the first fulcrum component part may be performed before a second fulcrum component part is assembled to the first fulcrum component part. In this case, the optical detector 1160 and optical waveguide 1154 may be assembled with the substrate structure 1156 also before assembling the second fulcrum component part with the first fulcrum component part. The electro-optical motion sensing component 1105 comprises a flexible circuit substrate 1156, as well as optical, electro-optical, and electrical components provided upon the flexible circuit substrate 1156. The optical and electro-optical components include, in this embodiment, an optical emitter such as a light-emitting diode (LED) 1158, an optical waveguide 1154 which may be a specially designed optical fiber component as described previously herein and constructed to enable optical power modulation techniques to be employed in the micro-motion sensing system 1100, and an optical detector 1160. Electronic components 1180 provided on the flexible circuit substrate 1156 may include functions of a microprocessor unit or “MPU” (such as the functions of MPU 462 described above in connection with FIG. 4), as well as control functions of the electro-optical components 1158, 1160.

The flexible circuit substrate 1156 in this embodiment is made up of three portions 1179, 1186, 1192, specifically, a first, stationary flexible circuit substrate portion 1179; a second, stationary flexible circuit substrate portion 1186; and a third, flexing flexible circuit substrate portion 1192. The first flexible circuit substrate portion 1179 remains stationary during operation of the module. Electronic components 1180 are provided upon the first flexible circuit substrate portion 1179. Interconnecting wires extend as needed within all portions 1179, 1186, 1192 of the substrate structure 1156 to make electrical connections between the various electrical and electro-optical components, as one of skill in the art would understand. The second, stationary flexible circuit substrate portion 1186 also remains stationary in that when assembled as previously described rests securely upon the top fulcrum surface 1182 of the fulcrum component 1104. The second, stationary flexible circuit substrate portion 1186 carries the optical detector 1160 and a first portion of the optical waveguide 1154 (roughly, one half of the optical waveguide 1154) which portion of the optical waveguide 1154 thus remains stationary during operation. The third flexible circuit substrate portion 1192, referred to herein as a flexing portion 1192, carries the optical emitter 1158 and the remaining portion (roughly one-half) of the optical waveguide 1156. The third flexible circuit substrate portion 1192 may be positioned within the module housing 1103, as in the case of this embodiment, so that the third flexible circuit substrate portion 1192 has sufficient open space beneath, namely, open chamber 1193, allowing the third flexible circuit substrate portion 1192 to flex downward in response to an external force applied from above. (Here, “top” and “bottom, as well as for example “beneath” and “downward,” are defined with respect to the orientation of the fulcrum component 1004 and electro-optical system 1005 as shown in FIGS. 10A and 10C.) A supporting leaf spring 1197 may be provided under a part of the second, stationary flexible circuit substrate portion 1186 and extending to, and under, a part of the third, flexing flexible circuit substrate portion 1192, as illustrated in FIGS. 11C and 11E. This supporting leaf spring 1197 supports, from underneath, the substrate 1156 and optical waveguide 1154 provided thereon, and provides a spring force that returns the third, flexing substrate portion 1192 and optical waveguide 1154 back to an original resting or less flexed position when a force causing the flexing is removed or reduced.

In further detail during operation, an inner surface 1151 of the button or pad structure 1150, in response to forces applied against the skin facing surface 1122 of the button or pad structure 1150 resulting from the presence of arterial or other waves in the underlying blood vessel, will bear similar forces against a side of the optical waveguide 1154 and/or against the flexible circuit substrate 1156 upon which the optical waveguide 1154 is positioned. The force applied against the waveguide 1156 and/or third, flexing substrate portion 1192 causes the third, flexing substrate portion 1192, as well as the portion of the optical waveguide 1154 carried thereon, to flex downward. As such, the optical output of the waveguide 1154 may be modulated in accordance with the principles of optical power modulation describe above.

As described previously in this document for example in connection with FIGS. 3A and 3B, modulation of the optical power output may be accomplished through flexing of the optical waveguide 1154, compression of the optical waveguide 1154 (which may in some embodiments be accomplished without the need for the third flexible circuit substrate portion 1192 and optical waveguide 1154 carried thereon to flex downward as described), or a combination of flexing and compression. In a case where optical power modulation is accomplished through compression, it may be advantageous as described previously in this document that the flexible circuit substrate structure 1156 generally be non-compressible as compared to the compressibility of the optical waveguide 1154, such that a force applied against a side surface of the optical waveguide 1154 results in compression of the waveguide 1154 structure and not the underlying substrate 1156.

The skin interfacing system 1106 has a generally cylindrical button or pad structure 1150 that extends through an opening 1155 in the bottom housing component 1101 so that a skin contacting surface 1122 of the button or pad structure 1150 may be held, when in use, against a subject's skin adjacent an underlying blood vessel. The skin contacting surface 1122 in this embodiment is generally flat in shape, although angled slightly to one side which may provide in some examples a better interface with the skin surface adjacent an underlying vessel. The interfacing component 1106 also has, opposite the skin contacting surface 1122, an inner surface 1151 that bears against the optical waveguide 1154 and/or flexible circuit substrate 1156 of the electro-optical motion sensing component 1174. In this embodiment, the inner contacting surface 1151 is provided on an inner half-cylinder structure 1194 which is provided at, and integral with, an inner portion of an outer cylindrical portion 1195 of the button or pad structure 1150. The inner half-cylinder structure 1194 includes the inner contacting surface 1151 and 1194 is oriented such that its longitudinal axis is generally perpendicular to a longitudinal axis of the upper cylindrical portion 1195.

A leaf spring 1152 fixedly attaches at one end to a side of the outer cylindrical portion 1195 of the button or pad structure 1150, and at an opposite end is fixedly attached to the bottom housing component 1101. The leaf spring 1152 is designed and configured to allow the button or pad structure 1150 to flex downward upon added force being applied to the button or pad structures skin facing surface 1122, and cause the button or pad structure 1150 to return toward a resting state (that is, the button or pad structure 1150 flexing back upward) when the force applied against surface 1122 reduces (downward and upward, as defined in the orientation of FIG. 11C). The fixation of the leaf spring 1152 to the bottom housing component 1101 may be provided by the leaf spring 1152 being fixedly secured into a horizontal channel 1196 formed in the top wall 1161, wherein the channel 1196 is formed in the side of a borehole of the top wall 1161 that provides for the cylindrical opening 1155.

Referring now to the bottom and top isometric views of FIGS. 11D and 11E, it can be seen that the flexing portion 1192 of the substrate component 1156, carrying a portion of an optical waveguide 1152 and an optical emitter 1158, is not provided in a “stacked” orientation with a stationary portion 1179 of the substrate component 1156 carrying electronic components 1180 such as processing circuitry, but instead are provided in two different lateral locations relative to the subject's skin when a device having the system 1100 is worn or applied against the skin as intended. As such, the micro-motion sensing system 1100 may enable a device into which the system 1100 is incorporated to have a lower, or more compact, profile relative to the surface of a subject's skin. Such a lower or more compact profile may be desirable in some cases in a wrist-wearable device, as an example.

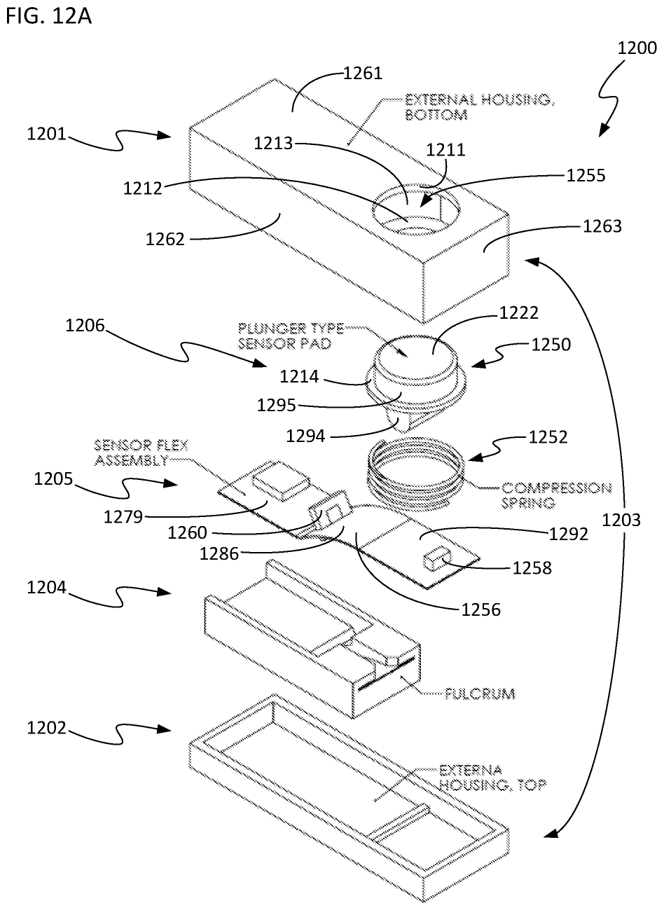

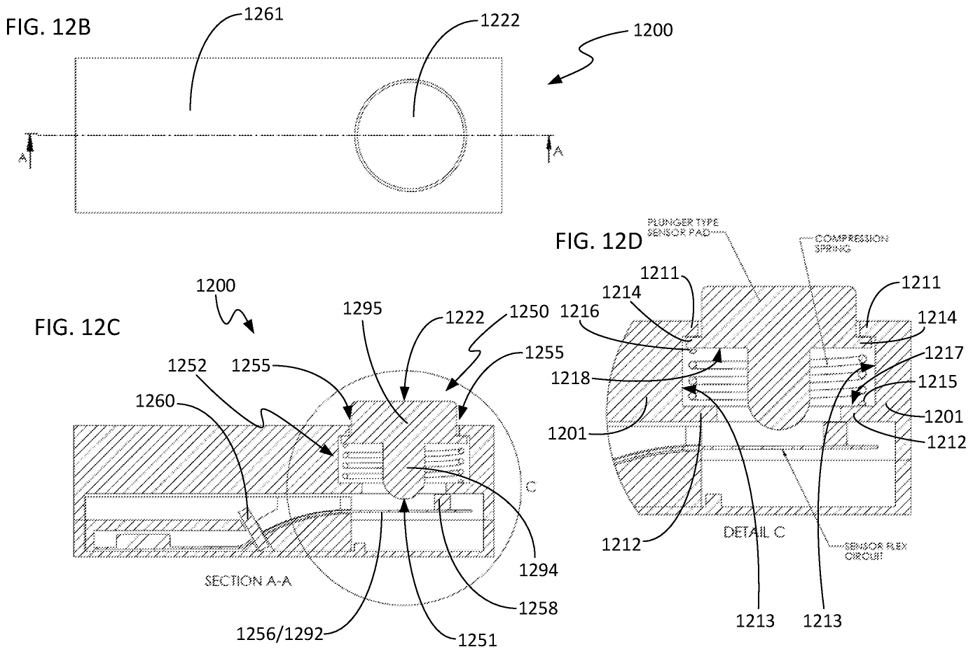

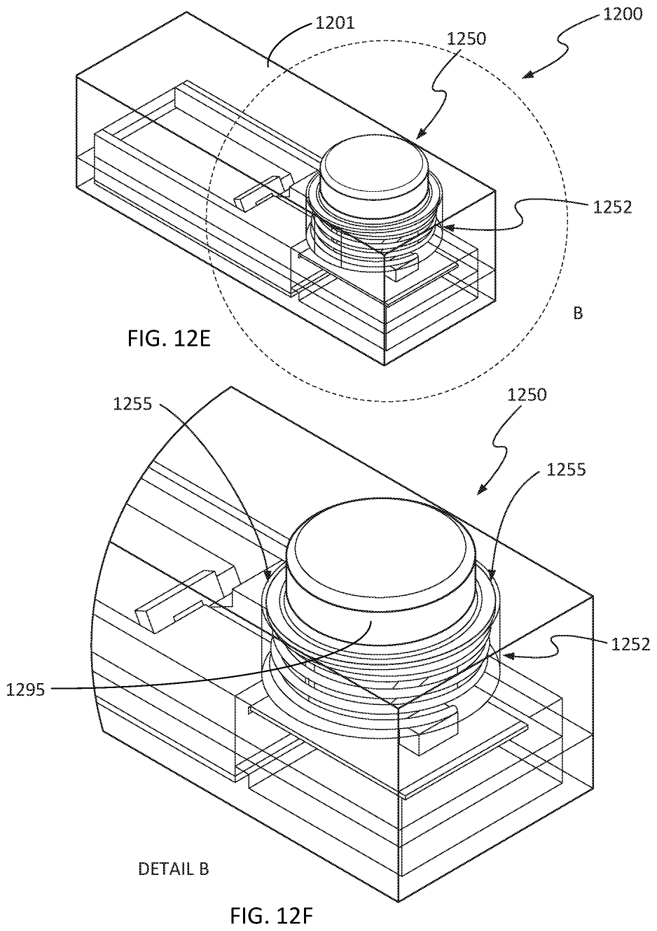

Turning now to FIGS. 12A-F, there is shown an additional design of a micro-motion sensing system 1200 adapted for use in non-invasive blood pressure monitoring devices, systems and methods, for example, in the devices and systems of FIGS. 1, 4, and 6-9. The non-invasive blood pressure monitoring systems and methods—in which the micro-motion sensing system 1200 may be utilized—may provide continuous, “beat-to-beat” measures of blood pressure without the need for an inflatable cuff and without the need for calibration of the systems or methods for a particular subject using a separate blood pressure measurement system. In the example shown in FIGS. 12A-F, the micro-motion sensing system 1200 utilizes optical power modulation techniques for micro-motion sensing, as described above in connection with FIGS. 2, 3A-B, and 4. The micro-motion sensing system 1200 of FIGS. 12A-F may be utilized in a blood pressure monitoring device adapted to be worn or applied to a skin surface of a subject, adjacent an underlying blood vessel, to obtain a continuous blood pressure measurement.

The sensing system 1200 of FIGS. 12A-F is similar in many respects to the sensing system 1000 of FIGS. 10A-E, except that the sensing system 1200 of FIGS. 12A-F employs a modified skin interfacing system 1206 and a modified and differently dimensioned bottom housing component 1201 as compared to the skin interfacing system 1006 and bottom housing component 1001 of the sensing system 1000 of FIGS. 10A-E. In some configurations, the design of the sensing system 1200 may provide various advantages, as compared, for example, to implementations of certain micro-motion sensing system designs described above and shown in FIGS. 6-8 and 10-11. One such advantage may be providing a design that enables options for water proofing or resistance that may be easier to implement.

In the previously described FIGS. 6-8 and 10-11 sensing system designs, a skin interfacing system includes a spring mechanism in the form of a leaf spring (652, 752, 852, 1052, 1152) that extends to the side of a button or pad assembly (750, 850, 1050, 1150) and is fixed to a bottom wall of the module's housing structure. By contrast, in the embodiment of the sensing system 1200 of FIGS. 12A-F, a coil spring 1252 is utilized instead of a leaf spring structure, to perform the functions described above that are performed by a leaf spring structure in the embodiments of FIGS. 6-8 and 10-11. In some implementations using a coil spring as implemented in the embodiment of FIGS. 12A-F, an inner half-cylinder portion 1294 of the button or pad structure 1250 may be elongated vertically to accommodate the coil spring 1252 being provided effectively within an outer portion of the button or pad structure 1250, as compared to the inner cylindrical portions of the button or pad structures of the FIGS. 6-8 and 10-11 designs. Additionally, the height of bottom housing component 1201 (that is, the height of the bottom housing component's side walls 1262, 1263) may be increased to accommodate an elongated inner pad or button portion 1294 and the coil spring 1252, as compared to the button or pad structure of the FIGS. 6-8 and 10-11 designs. The modified structure of the coil spring 1252 and related modifications to the bottom housing component 1201 and button or pad structure 1250 as illustrated in the FIG. 12A-F embodiment are applicable to not just the FIGS. 12A-E and 10A-E embodiments, but more broadly are applicable to a wide variety of sensing module embodiments.

This description will focus on those aspects of the FIG. 12A-F embodiment that differ from the embodiment of FIGS. 10A-F. Referring to FIG. 12A, the micro-motion sensing system 1200 is shown in a perspective view and exploded to illustrate better its components. FIG. 12B is a bottom side view of the system 1200. With regards to the orientation of “top” side and “bottom” side as used with respect to the housing components of the FIG. 12 embodiment, the “top” side is the side of the system that would be furthest from the skin and the “bottom” side is the side facing the skin, when a device incorporating the system is worn as intended. FIG. 12C is a longitudinal, vertical cross-sectional view of the system 1200, along the plane A-A defined in FIG. 12B, and FIG. 12D is a detailed view of a portion of FIG. 12E. FIG. 12E is a top isometric view of system 1200, and FIG. 12F is a detailed view of a portion of FIG. 12E.

As illustrated in the exploded view of FIG. 12A, the micro-motion sensing system 1200 includes two external housing components—a bottom housing component 1201 and a top housing component 1202—adapted to be connected to one another to form an external module housing 1203 having a cuboid shape. Housing component 1201 is referred to herein as a “bottom” housing component because it is located nearest the skin when a device incorporating the system 1000 is worn, whereas housing component 1202 is referred to herein as a “top” housing component because it would be the outer surface furthest to the skin when a device incorporating the system 1000 is worn. A fulcrum component 1204 resides within an internal chamber formed by the two housing components 1201, 1202, when connected to one another to form the system external housing 1203. A “flattened Z” shaped electro-optical motion sensing component 1205 is, in part, carried by, and engaged against, the fulcrum component 1204. A skin interfacing system 1206—configured on one side to bear against a surface of a subject's skin when in use and on an opposite side bear against an optical waveguide (not shown in FIGS. 12A-F, but would be positioned as shown in FIG. 10A) and/or a flexible circuit substrate 1256 underlying the optical waveguide, the substrate 1256 and optical waveguide being part of the electro-optical motion sensing component 1205—is fixed to the bottom housing component 1201 near an opening 1255 in the bottom housing component 1201.

In more detail, the bottom housing component 1201 has, in this embodiment, a cuboid shape, but in contrast to bottom housing component 1001 of FIGS. 10A-E, bottom housing component 1201 has a greater height dimension as mentioned to accommodate the coil spring 1252 effectively being between the button or pad structure 1250 and the skin interfacing system 1205 and accordingly the inner portion 1294 of the button or pad structure 1250 being elongated. As such, the bottom housing component 1201 has two generally flat, rectangular long side walls 1262 (the “long side” referring to a side of component 1201 that extends along the longest side dimension of the cuboid structure; only one of which long side walls 1262 being shown in FIG. 12A); and two generally flat, rectangular short side walls 1263 (the “short side” referring to a side of component 1201 that extends along the shortest dimension of the cuboid structure; only one of which short side walls 1263 being shown in FIG. 12A), wherein both sets of side walls 1262, 1263 are taller than the corresponding side walls 1062, 1063 of the FIG. 10A-E embodiment. A circular opening or borehole 1255 is provided in a bottom wall 1261 of the bottom housing component 1201, on one side of the bottom wall 1261, and positioned so that the generally cylindrically shaped button or pad structure 1250 of the skin interfacing component 1206 is aligned therewith so it is permitted to extend through the circular opening 1255 so that a skin contacting surface 1222 of the button or pad structure 1250 makes contact with, when in use as intended, the surface of the skin of a subject. The bottom housing component 1201 mates or interconnects with top housing component 1202 to form the system external housing 1203, as described for housing components 1001, 1002 in connection with the FIG. 10A-E embodiment.

The fulcrum component 1204 and electro-optical motion sensing system 1205 are identical in design to the corresponding fulcrum component 1004 and sensing system 1005 of the FIG. 10A-E embodiment, although the optical waveguide is not shown in FIGS. 12A-F but will be understood to be part of the electro-optical motion sensing system 1205 and would be positioned on a flexible circuit substrate 1256 extending between an optical emitter 1258 and an optical detector 1260 as with the FIG. 10A-E embodiment. The fulcrum component 1204 and electro-optical motion sensing system 1205 are intended to be assembled and placed within the top housing component 1202 as shown and described in connection with the FIG. 10A-E embodiment.

As with the FIG. 10A-E embodiment, the flexible circuit substrate 1256 is made up of three portions 1279, 1286, 1292, specifically, a first, stationary flexible circuit substrate portion 1279; a second, stationary flexible circuit substrate portion 1286; and a third, flexing flexible circuit substrate portion 1292. The first flexible circuit substrate portion 1279 remains stationary during operation of the system 1200. The second, stationary flexible circuit substrate portion 1286 also remains stationary when assembled and resting securely upon a top fulcrum surface of the fulcrum component 1204. The second, stationary flexible circuit substrate portion 1286 carries the optical detector 1260 and a first portion of the optical waveguide (not shown in FIGS. 12A-F, but roughly, one half of the optical waveguide), which portion of the optical waveguide thus remains stationary during operation. The third flexible circuit substrate portion 1292, referred to herein as a flexing portion 1292, carries the optical emitter 1258 and the remaining portion (roughly one-half) of the optical waveguide. The third flexible circuit substrate portion 1292 may be positioned within the module housing 1203, as in the case of this embodiment, so that the third flexible circuit substrate portion 1292 has sufficient open space beneath, allowing the third flexible circuit substrate portion 1292 to flex downward in response to an external force applied from above. (Here, “top” and “bottom, as well as for example “beneath” and “downward,” are defined with respect to the orientation of the fulcrum component 1004 and electro-optical system 1005 as shown in FIGS. 10A and 10C.) A supporting leaf spring may be provided under a part of the second, stationary flexible circuit substrate portion 1286 and extending to, and under, a part of the third, flexing flexible circuit substrate portion 1292, as described in connection with the FIG. 10A-E embodiment and illustrated in FIGS. 10C and 10E as supporting leaf spring 1097.

Referring to FIGS. 12A, 12C, and 12D, the skin interfacing system 1206 has a generally cylindrical button or pad structure 1250 that extends through an opening or borehole 1255 in the bottom housing component 1201 so that a skin contacting surface 1222 of the button or pad structure 1250 is held, when in use, against a subject's skin adjacent an underlying blood vessel. The skin contacting surface 1222 in this embodiment is generally flat in shape with beveled edges. The interfacing component 1206 also has, opposite the skin contacting surface 1222, an inner surface 1251 that bears against the optical waveguide (not shown) and/or flexible circuit substrate 1256 of the electro-optical motion sensing component 1274. In this embodiment, the inner contacting surface 1251 is provided on an inner half-cylinder structure 1294 which is provided at, and integral with, an inner portion of an outer cylindrical portion 1295 of the button or pad structure 1250. As compared to the button or pad structure 1050 of the FIG. 10A-E embodiment, the inner half-cylinder structure 1294 may be said to be an elongated half-cylinder structure 1294 in that the half-cylinder portion of the structure 1294 is at the bottom of a flange portion 1214 of the outer button or pad portion 1295 that is oriented parallel with the half-cylinder portion. The inner half-cylinder structure 1294 includes the inner contacting surface 1251 and is oriented such that its longitudinal axis is generally perpendicular to a longitudinal axis of the outer cylindrical portion 1295.

Referring to FIG. 12A as well as FIGS. 12C-D, the bottom housing component 1201 includes certain annular structures defining the opening 1255, including an outer annular flange 1211 adjacent a bottom surface of the bottom housing component 1201 (defining an outer portion of the opening 1255) and an inner annular flange 1212 adjacent an inside surface of the bottom housing component 1201 (defining an inner portion of the opening 1255), wherein annular flanges 1211, 1212 have an annular recessed region 1213 residing therebetween. As such, the outer and inner annular flanges 1211, 1212, along with the recessed region 1213, collectively define the opening or borehole 1255 in the bottom wall 1261 of the bottom housing component 1201. The button or pad structure 1250 includes an annular or outwardly extending shoulder 1214, which is located at an inner portion of the outer cylindrical portion 1295, or in other words, where the outer button or pad portion 1295 meets the inner button or pad portion 1294. The pad or button structure's annular shoulder 1214 is sized to reside entirely within the annular recessed region 1213 yet move inward and outward in piston-like fashion within the annular recessed region 1213. As such, piston-like movement of the button or pad structure 1250 within the top housing component's opening or borehole 1255 is provided.

The coil spring 1252, along with the button or pad structure's annular shoulder 1214, resides entirely within the annular recessed region 1213 of the opening or borehole 1255, inside the outer cylindrical portion 1295 of the button or pad structure 1250 and axially encircling the elongated inner half-cylinder portion 1294 of the button or pad structure 1250. Specifically, the coil spring 1252 is oriented to reside inside or within the outer cylindrical portion 1295 and axially encircling the elongated inner half-cylinder portion 1295 such that the coil spring's central longitudinal axis is co-extensive with central longitudinal axes of the opening or borehole 1255 of the button or pad structure 1250.

The coil spring 1252 comprises, in the FIG. 12A-F embodiment, approximately 3½ coils of spring structure, having a diameter that is slightly less than a diameter of the opening defined by the annular recessed region 1213, within which the coil spring 1252 resides, yet slightly greater than the diameter of the opening defined by the lower annular flange 1212 to which the coil spring 1252 is attached. One end of the coil spring 1252 fixedly attaches at a first connection point 1215 to the bottom housing component 1201, and an opposite end of the coil spring 1252 fixedly attaches at a second connection point 1216 to the button or pad structure. More specifically, the first connection point 1215 of spring attachment is provided upon an outer surface 1217 of the inner annular flange 1212, and the second connection point 1216 is provided on an inner surface 1218 of the outer cylindrical portion 1295 of the button or pad structure 1250, specifically the portion of the inner surface 1218 that is located on the annular shoulder 1214. The coil spring 1252 is designed and configured to allow the button or pad structure 1250 to flex inwardly upon added force being applied to the button or pad structures skin facing surface 1222, and cause the button or pad structure 1250 to return to a resting state (that is, the button or pad structure 1250 flexing back outward) when the force applied against surface 1222 reduces.

Regarding operation, an inner surface 1251 (labeled in FIG. 12C) of the button or pad structure 1250, in response to forces applied against the skin facing surface 1222 of the button or pad structure 1250 resulting from the presence of arterial or other waves in an underlying blood vessel, will bear against a side of the optical waveguide (not shown in FIGS. 12A-F, but positioned between the emitter 1258 and detector 1260 as described above) and/or against the third portion 1292 of the flexible circuit substrate 1256 upon which the optical waveguide is positioned. The force applied against the waveguide and/or third, flexing substrate portion 1292 causes the third, flexing substrate portion 1292, as well as the portion of the optical waveguide carried thereon, to flex inwardly (downward) and/or compress. As such, the optical output of the waveguide may be modulated in accordance with the principles of optical power modulation as described above.

As described previously in this document, for example in connection with FIGS. 3A and 3B, modulation of the optical power output may be accomplished through flexing of the optical waveguide, compression of the optical waveguide (which may in some embodiments be accomplished without the need for the third flexible circuit substrate portion 1292 and optical waveguide carried thereon to flex downward as described), or a combination of flexing and compression. In a case where optical power modulation is accomplished through compression, it may be advantageous as described previously in this document that the flexible circuit substrate structure 1256 generally be non-compressible as compared to the compressibility of the optical waveguide, such that a force applied against a side surface of the optical waveguide results in compression of the waveguide structure and not the underlying substrate 1256.

Referring now to the bottom isometric views of FIGS. 12E-F, it is seen that the coil spring 1252 resides entirely within an opening or borehole 1255 of the bottom housing component 1201, within (in other words, below or underneath in the orientation of FIGS. 12E-F) an outer cylindrical portion 1295 of the button or pad structure 1250. As constructed, in some embodiments designs incorporating such features of the sensing system 1200 of FIGS. 12A-F may be constructed to be more resistant to water entering the house through the borehole 1255, making it easier to configure the sensing system 1200 to be waterproof or water resistant, as compared to other embodiments of sensing modules.

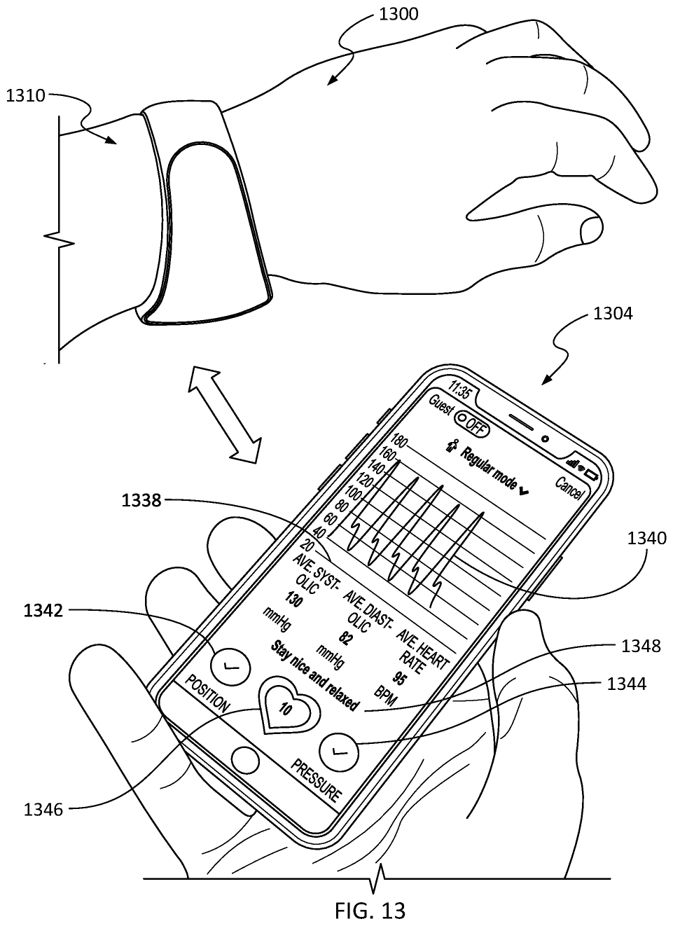

Referring now to FIG. 13, there is shown a wrist-worn device 1300 being worn on the wrist 1310 of a human subject, along with a local device 1304 in the form of a smartphone device provided with a specially designed blood pressure monitoring application program designed for use with the wrist-worn device 1300. Briefly as shown in FIG. 13, the local device 1304 includes a user interface visual display 1338 providing various information relating to the monitoring of blood pressure by the wrist-worn device 1300. For example, the display 1338 includes a continuous beat-to-beat sensor waveform 1340 configured to be displayed as the wrist-worn device provides the information and so that the waveform scrolls across the display 1338 from left to right. The display 1338 also provides numerical read-outs located near the center of the display 1338, for various average blood pressure measures (for example, averages over ten cardiac cycles), including in this example average systolic pressure, average diastolic pressure, and average heart rate.

Near the bottom of the display 1338, there is provided a “position” indicator 1342, which indicates whether or not the positioning is correct or not for the wrist-worn device 1300 so that a skin-contacting portion of the device 1300 is positioned correctly on the skin vis-à-vis the underlying artery. The correctness of the positioning may be indicated with a check mark and appropriate green coloring as shown in FIG. 13 if the positioning is correct, or alternatively with an “X” mark and red coloring if the positioning is not correct. Also located near the bottom of the display is a “force” indicator 1344, which indicates whether or not the hold-down force of the wrist-worn device 1300 is within an acceptable range, for example, within 5-15 mm Hg or some other appropriate range as discussed above. Here too, the correctness of the hold-down force may be indicated with check mark and appropriate green coloring as shown in FIG. 13 if the hold-down force is correct or in other words within the proper range, or alternatively with an “X” mark and red coloring if the hold-down force is not correct or in other words is outside of the proper range. Also near the bottom of the display 1338 is a timer device 1346 shaped like a heart and having a number indicating the number of seconds that the device 1300 has been taking a blood pressure reading. A typical reading of continuous measures may be a 30-second period, for example. Above the timer 1346 is a message box 1348, which in this example reads “Stay nice and relaxed,” given as shown because the device 1300 is in the process of taking a continuous blood pressure measurement reading.

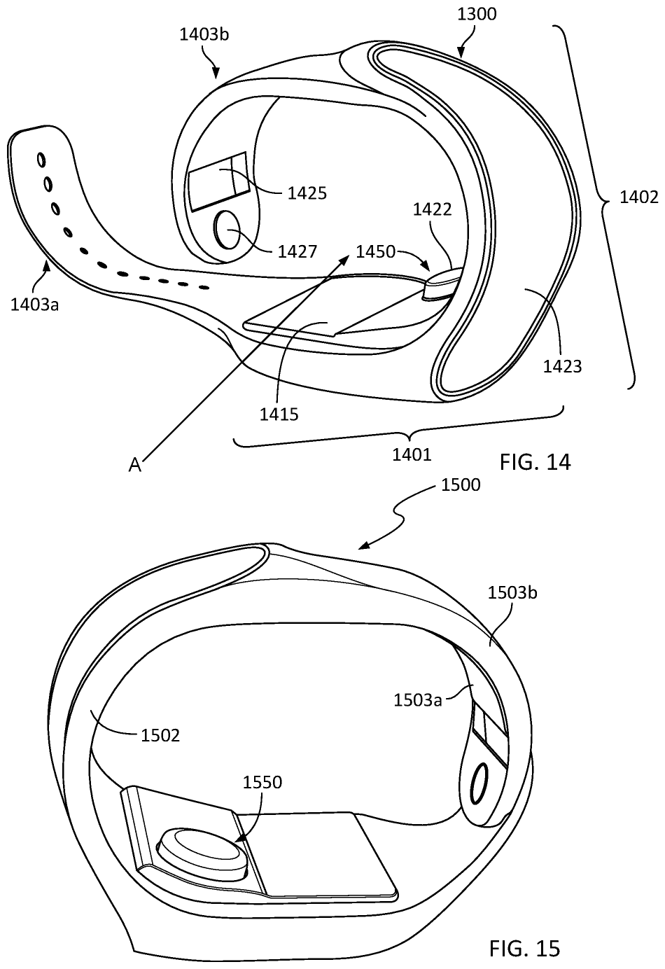

FIG. 14 is a perspective diagram of the wrist-worn blood pressure monitoring device 1300 shown in FIG. 13. The device 1300 includes a blood pressure monitoring portion 1401, which when worn has an inner surface that rests against the underside of the wrist as shown in FIG. 13. The blood pressure monitoring portion 1401 includes a micro-motion sensor contained within, the structure of which is described with respect to FIGS. 1 through 12 (and also in the '120 provisional patent application). Generally, the monitoring portion 1401 of the device 1300 includes a sensor housing 1415 within which the micro-motion sensor is housed. The housing 1415 is exposed at an inner surface of the blood pressure monitoring portion 1401 of the device 1300. The monitoring portion 1401 is otherwise covered by a rubber material, which may be generally rigid yet supple enough to adapt to individual wrist shapes, and which extends around the other portions of the micro-motion sensor that are not exposed. The micro-motion sensor includes a button or pad structure 1450 whose external skin-contacting surface 1422 protrudes from the sensor housing 1415, or in other words, the skin-contacting surface 222 protrudes from an inner surface of the device 1300 in the region of the blood pressure monitoring portion 1401. The button or pad structure 1450 is configured and positioned so that the skin-contacting portion 1422 of the button or pad structure 1450 may be positioned against a surface of the skin adjacent the radial artery. Internally within the housing 1450, an internal surface of the button or pad structure 1450, opposite the skin-contacting surface 1422, may bear against a side of an optical waveguide that is a component of the micro-motion sensor. As such, a force applied to the skin-contacting surface 1422 of the button or pad structure 1450 is translated to a force acting on the side of an internal optical waveguide, and as such the micro-motion sensor is able to measure in very fine increments the motion at the surface of the skin adjacent the artery when the device 1300 is positioned on the wrist, as is more completely with respect to FIGS. 1 through 12 (and also described in the '120 provisional patent application).

The button or pad structure 1450 in this example is positioned so that the device 130 is a left-hand device, owing to the button or pad structure 1450 being positioned on the inner surface so that when worn it is nearest the wearer's thumb. In particular, when the device 1300 is worn with the wearer's left arm, from forearm to hand, extending through the wrist-worn device 1300 in the direction of arrow A, with the underside of the wrist facing down, the skin-contacting portion 1422 of the button or pad structure 1450 will rest against a portion of the wearer's skin that is adjacent the radial artery, in an optimal location to be able to measure motion and thus blood pressure in accordance with the teachings presented with respect to FIGS. 1 through 12 (and also in the '120 provisional patent application). In other words, this is the location of the wrist where one may typically feel for a pulse.

The device 1300 includes, at a location immediately next to the blood pressure monitoring portion 1401, a generally rigid side portion 1402. When worn, the side portion 1402 rests against a side of the wrist that is closest to the thumb. The side portion may be generally rigid as in this embodiment so as to contribute to the accurate and consistent positioning of the micro-motion sensor against the skin surface adjacent the wearer's radial artery, yet sufficiently supple to enable the side portion 1402 of the device 1300 to be wrapped around a wrist and be adapted to individual users with varying wrist anatomies. The side portion may be made of a hard rubber material that is integral with the hard rubber material of the blood pressure monitoring portion 1401. A decorative outer plate 1423 may be embedded in an outer surface of the side portion 1402. In this example, the outer plate 223 may be a metal or a material that appears to be metal.

The device includes two straps, namely, a first strap 203a that is connected to the monitoring portion 1401, which when worn wraps around a side of the wrist nearest the wearer's pinky finger, and a second strap 1403b that is connected to the side portion 1402, which when worn wraps around a top side of the wrist. The straps may be made of the same rubber material and be integrally manufactured with the rubber portions of the monitoring portion 1401 and the side portion 1402 of the device. To secure the straps 1403a, 1403b together, the first strap 1403a is extended through an opening 1425 in the second strap 1403b, which opening 1425 is located near the distal end of the second strap 1403b. In particular, the first strap 1403a is extended through the opening 1425 from the outside, so that a distal end portion of the first strap 1403a is positioned against an inner surface of the second strap 1403b. The straps 1403a, 1403b may be fastened together with a knobbed post and hole configuration. As shown, the first strap 1403a has a series of holes positioned along the length of the strap 1403a, and extending entirely through the strap 1403a. The second strap 1403b includes a knobbed post (not shown in FIG. 14, but located on an outside of the strap 1403b opposite the post fastener 1427 shown on the inside of the second strap 1403b), and the knobbed post extending outward may be extended through an appropriate hole in the first strap 1403b, depending on the size of the wrist of the wearer. The inner surface of the second strap 1403b may have an indented portion provided therein and configured and sized so that a distal portion of the first strap 1403a may be placed within the indented portion. As such, the second strap 1403b in this example is wider than the first strap 1403a, so that the indented portion on the inner surface of the second strap 1403b accommodates a distal portion of the first strap 203a within the indented portion.

FIG. 15 shows a monitoring device 1500 that is the same design as the device 1500 shown in FIGS. 13-14, except that the device 500 is in a different color (white instead of black). The device 1500 of FIG. 15 is similarly a left-hand device. As shown, a wearer's wrist, from forearm to hand, would extend through the device 1500 from the backside of FIG. 15, so the thumb-side of the wrist is located against an inner surface of side portion 1502, and the button 1550 would rest against a skin surface adjacent the radial artery.

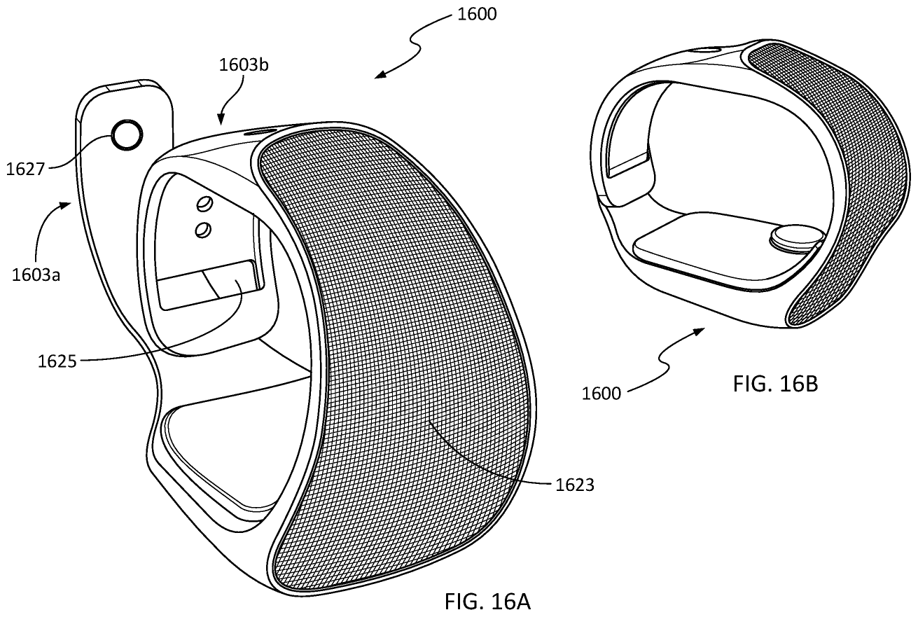

FIGS. 16A-B show another embodiment of a wearable monitoring device 1600, which is the same as devices 1300 and 1500 in FIGS. 13-15 except for the strap configuration. Unlike devices 1300 and 1500, the knobbed post (opposite of post fastener 1627) is provided on an outer surface of the first strap 1603a, and the series of holes are provided on the second strap 1603b. As with devices 1300 and 1500, the first strap 1603a is extended through the opening 1625 in the second strap 1603b from the outside, and a distal portion of the first strap 1603a may be placed within an indented portion provided in an inner surface of the second strap 1603b. As such, the knobbed post provided on the outside of the first strap 1603a may be extended through an appropriate hole in the second strap 1603b, depending on the size of the wearer's wrist. The decorative outer plate 1623 of the side portion of device 1600 has a fabric type design, unlike the smooth designs of the outer plate of devices 1300 and 1500.

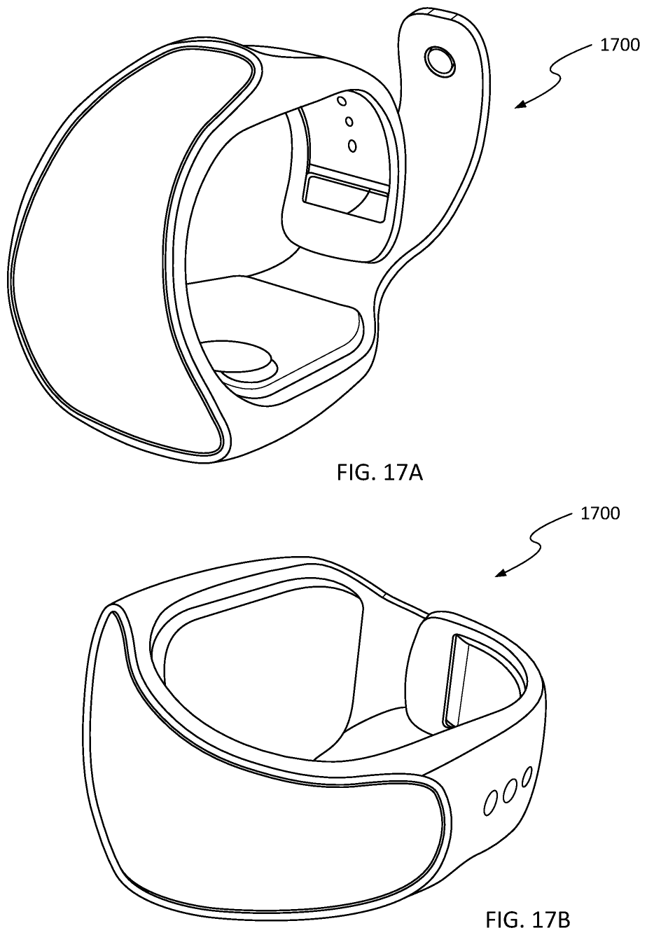

FIGS. 17A-B shows a device 1700 identical to the device 1600 of FIGS. 16A-B, except for the coloring (white instead of black) and the design of the decorative outer plate, which has a smooth gold-colored finish.

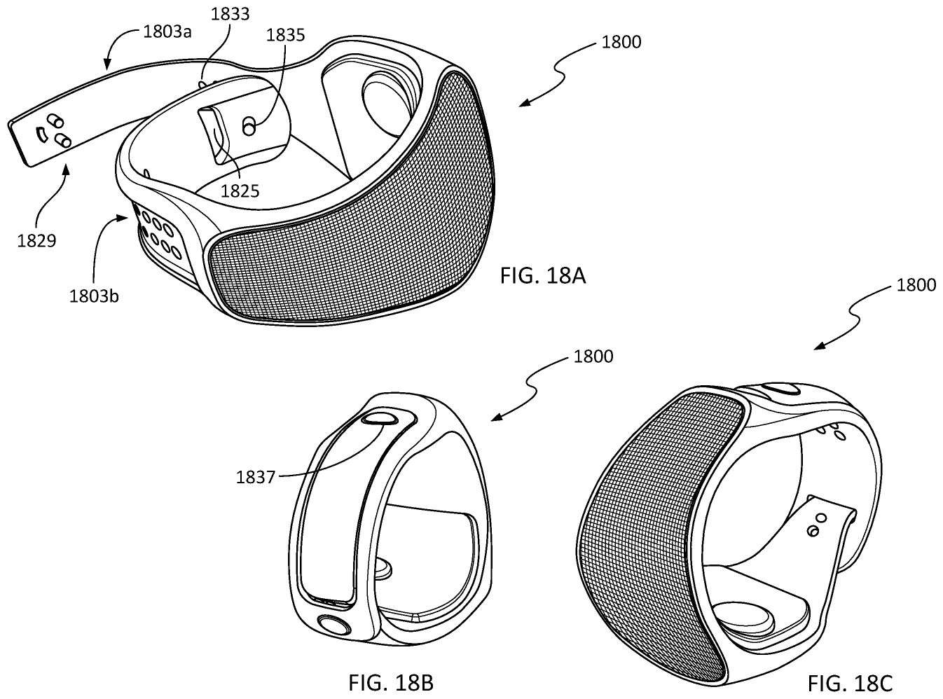

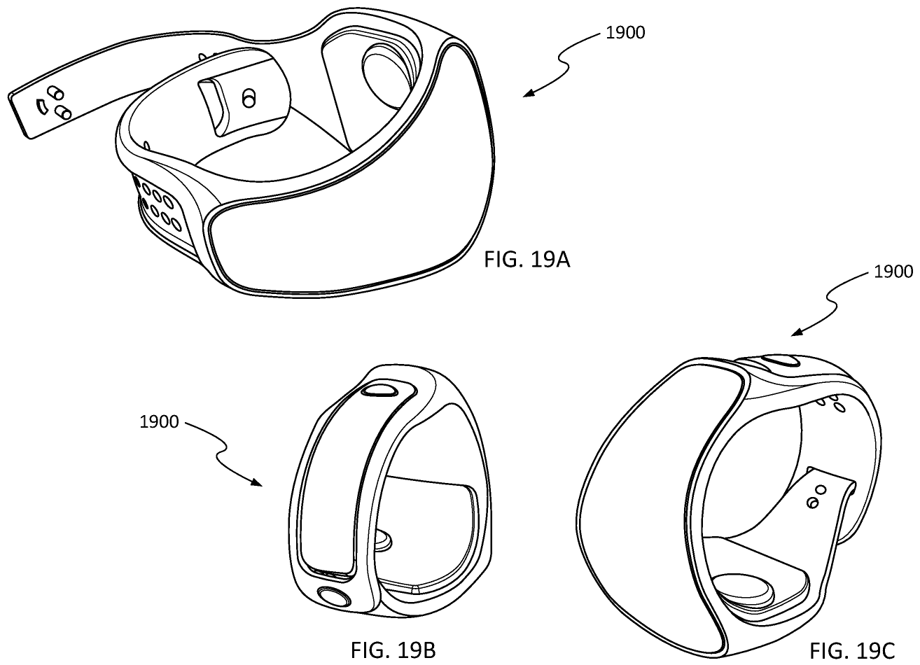

FIGS. 18A-C show another embodiment of a wearable monitoring device 1800, which is the same as previous embodiments except that it has yet another different strap configuration. In this device 1800, the first strap 1803a is extended through the opening 1825 in the second strap 1803b from the inside out, so that a distal portion of the first strap 1803a is mated into a corresponding indention on the outside of the second strap 1803b. As such, the second strap 1803b is wider than the first strap 1803a. In this embodiment, the distal end portion of the first strap 1803a is provided with two side-by-side knobbed posts 1829 (opposite post fastener 1837 having a decorative look as shown in FIG. 19B, and located on the outside of the first strap 1803a at near its distal end), and the second strap 1803b is provided with corresponding two rows of holes extending through the second strap 1803b. The first strap 1803a is also provided with a row of holes 1833 on a proximal portion of the first strap 1803a, and the second strap 1803b has a corresponding knobbed post on the outside of the strap 1803b distal of the strap's opening 1825 (the knobbed post being opposite the post fastener 1835 shown in the inner surface of the distal end of the second strap 1803b).

FIGS. 19A-C show a device 1900 identical to the device 1800 of FIGS. 18A-C, except for the coloring (white instead of black) and the design of the decorative outer plate, which has a smooth light gold-colored finish.

Mobile Device Program for Non-Invasive Continuous Blood Pressure Monitoring

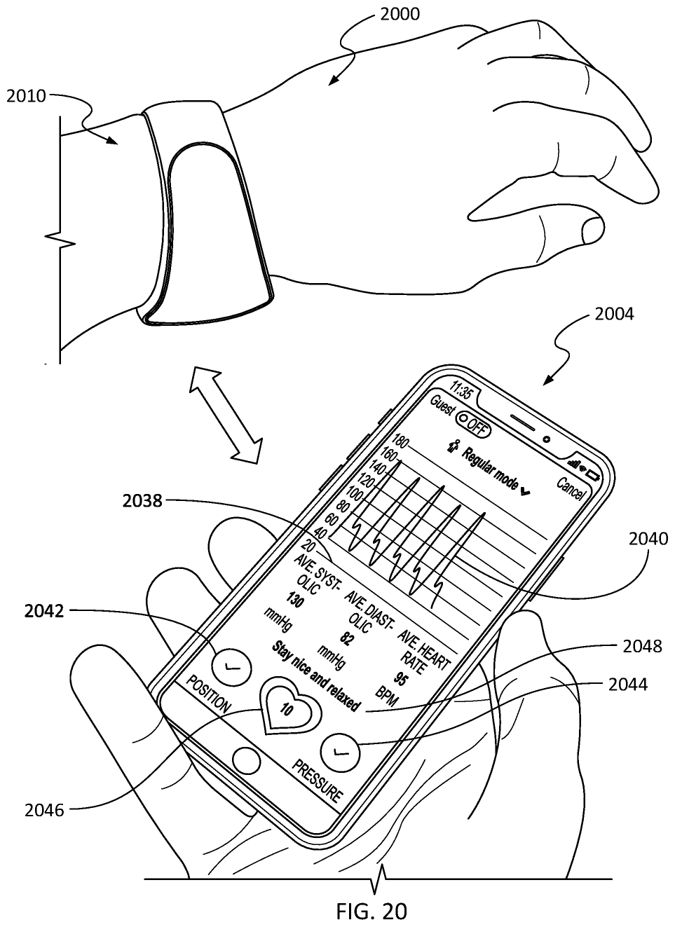

Referring now to FIG. 20, there is shown a wrist-worn device 2000 being worn on the wrist 2010 of a human subject, along with a local device 2004 in the form of a smartphone device provided with a specially designed blood pressure monitoring application program designed for use with the wrist-worn device 2000. Briefly as shown in FIG. 20, the local device 2004 includes a user interface visual display 2038 (similar to that shown in FIG. 20) providing various information relating to the monitoring of blood pressure by the wrist-worn device 2000. For example, the display 2038 includes a continuous beat-to-beat sensor waveform 2040 configured to be displayed as the wrist-worn device provides the information and so that the waveform scrolls across the display 2038 from left to right. The display 2038 also provides numerical read-outs located near the center of the display 2038, for various average blood pressure measures (for example, averages over ten cardiac cycles), including in this example average systolic pressure, average diastolic pressure, and average heart rate.

Near the bottom of the display 2038, there is provided a “position” indicator 2042, which indicates whether or not the positioning is correct or not for the wrist-worn device 2000 so that a skin-contacting portion of the device 2000 is positioned correctly on the skin vis-à-vis the underlying artery. The correctness of the positioning may be indicated with a check mark and appropriate green coloring as shown in FIG. 20 if the positioning is correct, or alternatively with an “X” mark and red coloring if the positioning is not correct. Also located near the bottom of the display is a “force” indicator 2044, which indicates whether or not the hold-down force of the wrist-worn device 2000 is within an acceptable range, for example, within 5-15 mm Hg or some other appropriate range as discussed above. Here too, the correctness of the hold-down force may be indicated with check mark and appropriate green coloring as shown in FIG. 20 if the hold-down force is correct or in other words is within the proper range, or alternatively with an “X” mark and red coloring if the hold-down force is not correct or in other words is outside of the proper range. Also near the bottom of the display 2038 is a timer device 2046 shaped like a heart and having a number indicating the number of seconds that the device 2000 has been taking a blood pressure reading. A typical reading of continuous measures may be a 30-second period, for example. Above the timer 2046 is a message box 2048, which in this example reads “Stay nice and relaxed,” given as shown because the device 2000 is in the process of taking a continuous blood pressure measurement reading.

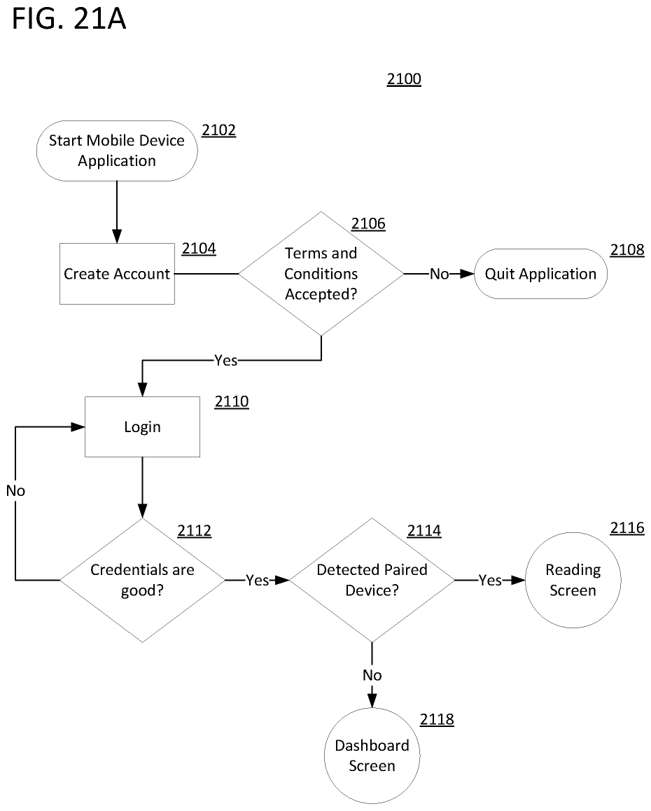

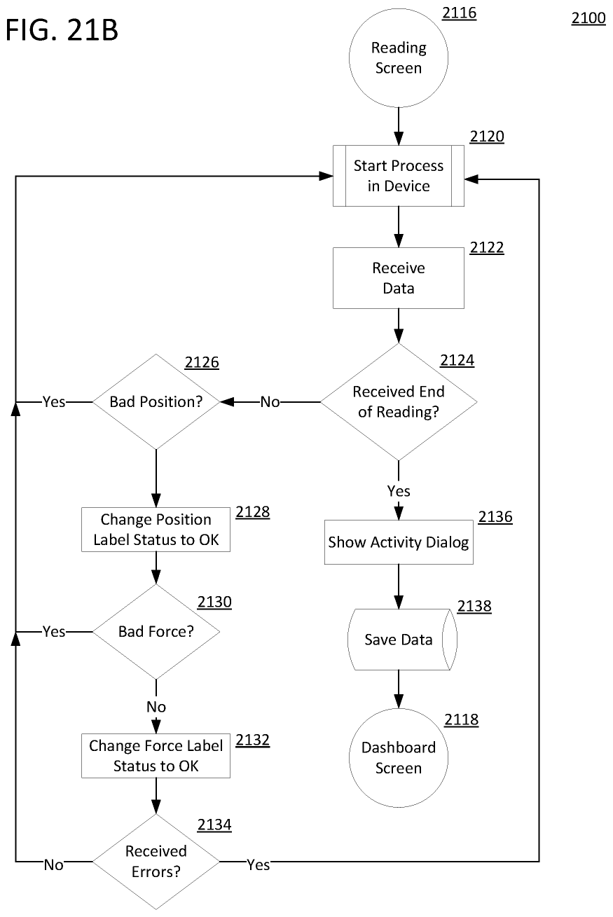

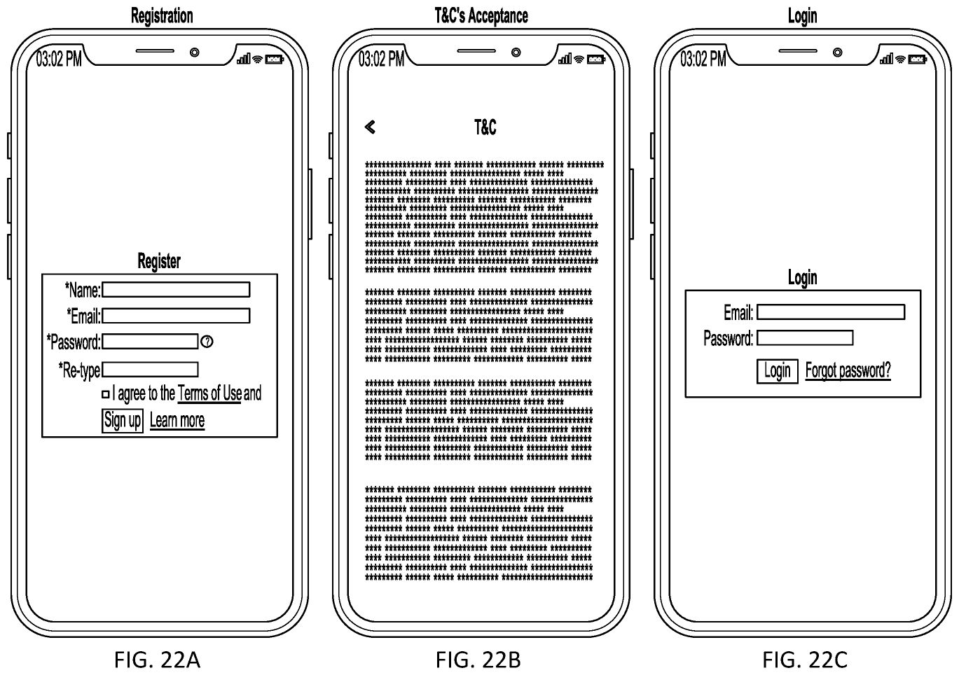

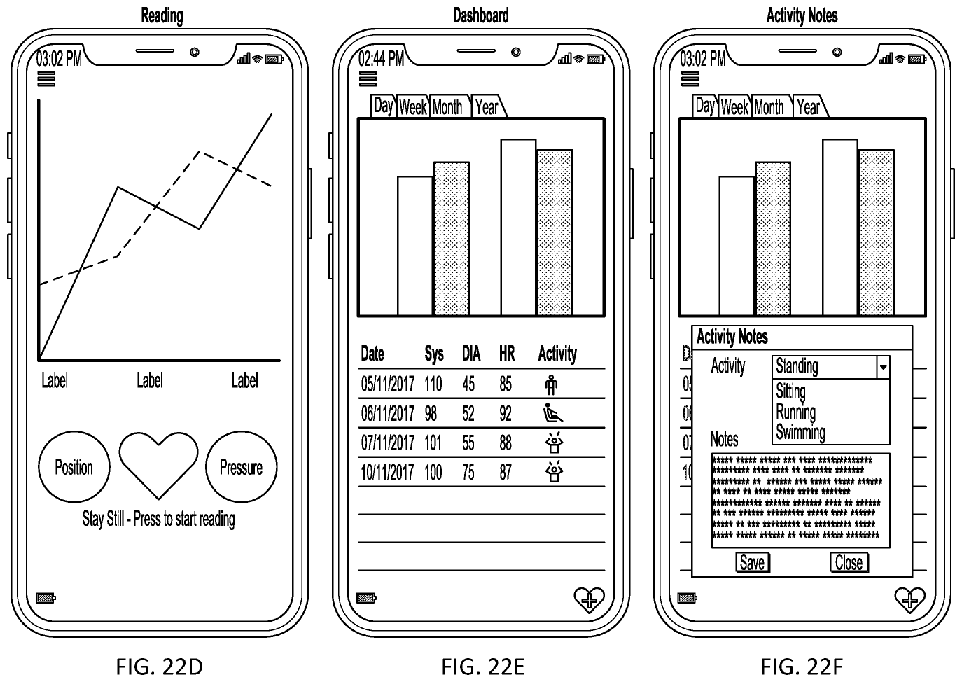

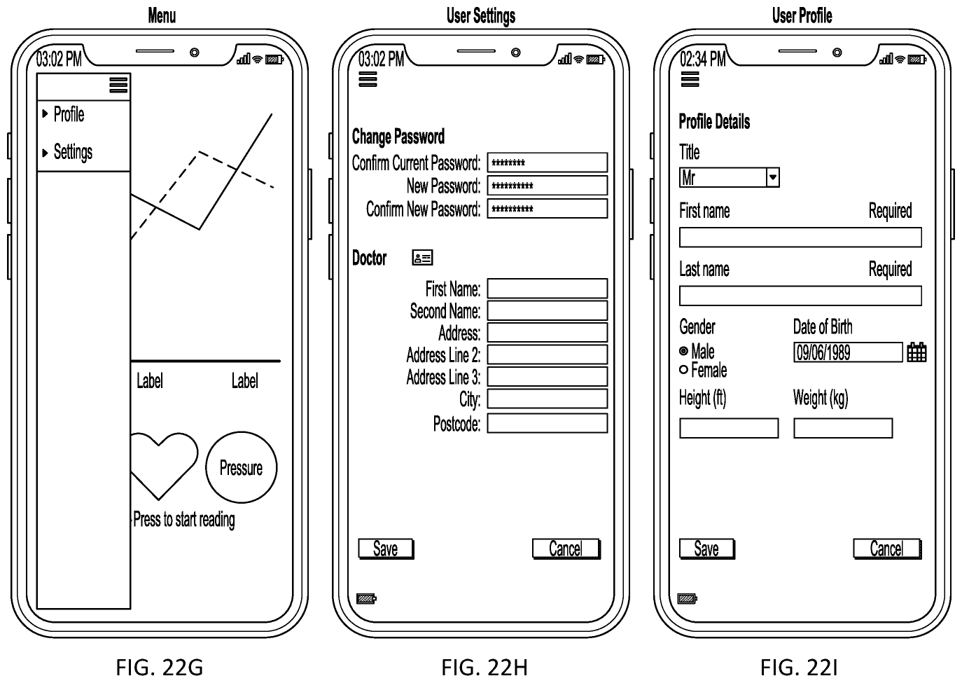

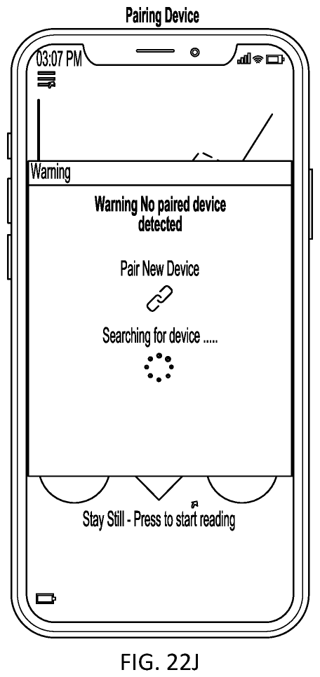

FIGS. 21A-21B are two parts of a flowchart describing the operation of a smartphone program application used in connection with a blood pressure monitoring device. In describing the flowchart, reference will be made to FIGS. 22A-J, which show an embodiment of a series of screens generated by a smartphone program application used in connection with a blood pressure monitoring device. At 2102, the mobile device application is started, and proceeds to 2104 to create an account. FIG. 22A illustrates an example of how an account may be created. FIG. 22A shows a registration window, enabling a user to register by providing a name, e-mail address, and password. A checkbox for agreeing to “terms of use” is also provided in the window shown in FIG. 22A, along with a “sign up” box, presentation and use of which corresponds to decision box 2106 of FIG. 21A. The terms and conditions may be displayed to the user in a separate window as shown in FIG. 22B. If at 2106 of FIG. 21A the terms and conditions are not accepted by the user (for example, by checking the box and hitting “sign up” in the window shown in FIG. 22A), the application ends at 2108. If accepted, the application proceeds to a login window at 2110. An example of a login screen is shown in FIG. 22C, which provides a screen wherein the user may enter an e-mail and password, and select a “Login” button.

After the selection of the “Login” button (for example, on the screen shown in FIG. 22C), the credentials entered are checked at 2112 under the flowchart of FIG. 21A to determine if the credentials are valid or not valid. If not valid, the user is returned to login at 2110 (for example, the screen at FIG. 22C). If valid, then the application proceeds to determine if a paired device is detected, at 2114 (FIG. 21A). If there is a paired device, the application proceeds to a reading screen 2116, wherein blood pressure information from a paired device may be presented. If there is not a paired device, the application may proceed to a dashboard screen at 2118 for the user to view previously recorded blood pressure information or perform other operations. An example of such a dashboard screen is provided in FIG. 22E, which provides historical information about previous blood pressure measurements that have been taken. At the top half of the dashboard screen in FIG. 22E, there is provided bar chart information for blood pressures recorded over various time periods as indicated by the tabs (daily, weekly, monthly, and yearly). At the bottom half of the dashboard screen shown in FIG. 22E, there is provided the results of the past four blood pressure measurements taken.

Referring now to FIG. 21B, and specifically the reading screen also referenced in FIG. 21A, a start process is performed at 2120 in the wearable device, for example, in a wrist-worn device 1300 as shown in FIG. 13. The smartphone device now starts to receive data at 2122 from the monitoring device such as device 1300 shown in FIG. 13. Data received at 2122 may include, as described previously with respect to FIGS. 1 through 12 and in the '120 provisional patent application, a continuous digitized sensor waveform along with beat-to-beat blood pressure measures for each cardiac cycle represented in the continuous sensor waveform. While the data is being received, continuously updated information about the blood pressure measurements may be displayed, for example as shown in FIG. 22D. FIG. 22D shows that the sensor waveform may be displayed in graphical format. In addition, the reading window of FIG. 22D may also indicate by the two circles in the lower portion of the display, the state of the positioning of the device and the state of the hold-down force. As shown in FIG. 22D, both are good by virtue of the “Position” and “Force” circles being colored green, which indicates to the user that the device is correctly positioned and has the correct hold-down force being applied.

Data may continue to be received by the smartphone device from the wearable device 1300 until an “end of reading” indication is provided, as determined at 2124 of FIG. 21B. This “end of reading” indication may be the timing out of a predefined period of time, for example, 30 seconds, in the case for example that the device is programmed to take a continuous blood pressure measurement for thirty seconds. Alternatively, the user may end the reading by either making an entry on the smartphone device or activating an input on the device 1300 itself. If at 2124 of FIG. 21B it is determined that an “end of reading” indication has not yet been received, the device cycles through its error checks that are made during the reading process. For example, at 2126 there is a check for whether the positioning is bad. If the positioning is bad, the application proceeds to start the process in the device at 2120 again. If positioning is not bad (it is good or continues to be good), then the positioning label status is changed to “OK” (such as a green color for the “Position” circle in FIG. 22D), and the application proceeds to a hold-down force check at 2130. As described with respect to FIG. 5 and in the '120 provisional patent application, the device 1300 may assess the hold-down force by analyzing the micro-motion sensor analog output signal to see if that signal is in a proper range, and the device 1300 may transmit an indication of whether or not the hold-down force is good or not good (in other words, within an acceptable range, for example of 5-15 mm Hg, or outside that range) to the smartphone device. If the pressure is bad as determined at 2130, the application proceeds to start the process in the device at 2120 again. If positioning is not bad (it is good or continues to be good), then the pressure label status is changed to “OK” (such as a green color for the “Force” circle in FIG. 22D), and the application proceeds to a check whether any errors have been received at 2134. If there are no errors received, the application proceeds to start the process in the device at 2120 again. If errors are received, the application also proceeds to start the process in the device at 2120 again. Possible errors may be that the device 1300 has gone out of wireless transmission range, a message is received from the device 1300 that the subject has been too active during a monitoring period (as determined for example by an activity sensor that may be provided in the device), or any number of other possible errors.

If at 2124 (FIG. 21B) it is determined that an “end of reading” indication has been received, that ends the blood pressure monitoring process, and the application proceeds to show an activity dialog at 2136. An example of such a dialog is shown in FIG. 22F, which shows a pop-up box being provided, in which the user is to enter a note about the activity being done if any while the blood pressure monitoring process was being carried out. As shown, the user may enter an activity describing, for example, that the user was standing, sitting, running, swimming, etc. Next, the application proceeds to save the data, including the data collected from the device 1300 during the monitoring process (at 2122 of the FIG. 21B flowchart), along with the user entering information regarding an activity from 2136. After that is done, the application proceeds to the dashboard screen at 2118. As has been previously described, an example of such a dashboard screen is shown in FIG. 22E.

Referring now to FIG. 22G, an application window shows how a menu may be provided to navigate to a profile page for the user or to settings for the application. In FIG. 22H, there is an example of a window that may be displayed with various user settings, with the option to save newly entered data or cancel the saving of the data. FIG. 221 shows an application window for a user profile, including data such as gender, age (from date of birth), height, and weight. Finally, FIG. 22J shows an application window that may be displayed during a pairing process to pair a device, such as the device 1300 from FIG. 13, to the smartphone device.

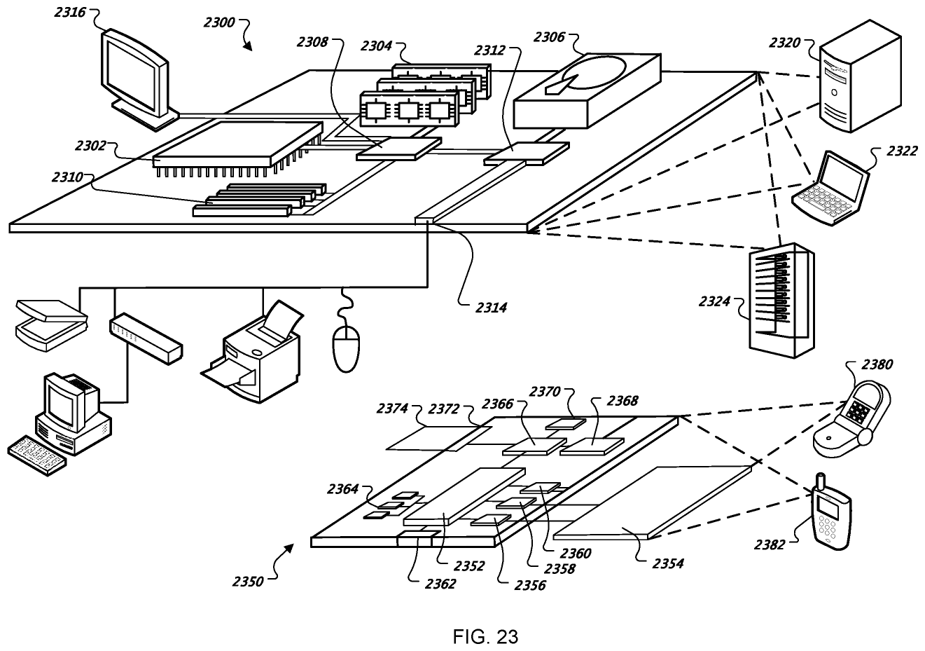

FIG. 23 is a block diagram of computing devices 2300, 2350 that may be used to implement the systems and methods described in this document, as either a client or as a server or plurality of servers. Computing device 2300 is intended to represent various forms of digital computers, such as laptops, desktops, workstations, personal digital assistants, servers, blade servers, mainframes, and other appropriate computers. Computing device 2350 is intended to represent various forms of mobile devices, such as personal digital assistants, cellular telephones, smartphones, and other similar computing devices. The components shown here, their connections and relationships, and their functions, are meant to be examples only, and are not meant to limit implementations described and/or claimed in this document.

Computing device 2300 includes a processor 2302, memory 2304, a storage device 2306, a high-speed interface 2308 connecting to memory 2304 and high-speed expansion ports 2310, and a low speed interface 2312 connecting to low speed bus 2314 and storage device 2306. Each of the components 2302, 2304, 2306, 2308, 2310, and 2312, are interconnected using various busses, and may be mounted on a common motherboard or in other manners as appropriate. The processor 2302 can process instructions for execution within the computing device 2300, including instructions stored in the memory 2304 or on the storage device 2306 to display graphical information for a GUI on an external input/output device, such as display 2316 coupled to high-speed interface 2308. In other implementations, multiple processors and/or multiple buses may be used, as appropriate, along with multiple memories and types of memory. Also, multiple computing devices 2300 may be connected, with each device providing portions of the necessary operations (e.g., as a server bank, a group of blade servers, or a multi-processor system).

The memory 2304 stores information within the computing device 2300. In one implementation, the memory 2304 is a volatile memory unit or units. In another implementation, the memory 2304 is a non-volatile memory unit or units. The memory 2304 may also be another form of computer-readable medium, such as a magnetic or optical disk.

The storage device 2306 is capable of providing mass storage for the computing device 2300. In one implementation, the storage device 2306 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 2304, the storage device 2306, or memory on processor 2302.

The high-speed controller 2308 manages bandwidth-intensive operations for the computing device 2300, while the low speed controller 2312 manages lower bandwidth-intensive operations. Such allocation of functions is an example only. In one implementation, the high-speed controller 2308 is coupled to memory 2304, display 2316 (e.g., through a graphics processor or accelerator), and to high-speed expansion ports 2310, which may accept various expansion cards (not shown). In the implementation, low-speed controller 2312 is coupled to storage device 2306 and low-speed expansion port 2314. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 2300 may be implemented in a number of different forms, as shown in the figure. For example, it may be implemented as a standard server 2320, or multiple times in a group of such servers. It may also be implemented as part of a rack server system 2324. In addition, it may be implemented in a personal computer such as a laptop computer 2322. Alternatively, components from computing device 2300 may be combined with other components in a mobile device (not shown), such as device 2350. Each of such devices may contain one or more of computing device 2300, 2350, and an entire system may be made up of multiple computing devices 2300, 2350 communicating with each other.

Computing device 2350 includes a processor 2352, memory 2364, an input/output device such as a display 2354, a communication interface 2366, and a transceiver 2368, among other components. The device 2350 may also be provided with a storage device, such as a microdrive or other device, to provide additional storage. Each of the components 2350, 2352, 2364, 2354, 2366, and 2368, are interconnected using various buses, and several of the components may be mounted on a common motherboard or in other manners as appropriate.

The processor 2352 can execute instructions within the computing device 2350, including instructions stored in the memory 2364. The processor may be implemented as a chipset of chips that include separate and multiple analog and digital processors. Additionally, the processor may be implemented using any of a number of architectures. For example, the processor may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 2350, such as control of user interfaces, applications run by device 2350, and wireless communication by device 2350.

Processor 2352 may communicate with a user through control interface 2358 and display interface 2356 coupled to a display 2354. The display 2354 may be, for example, a TFT (Thin-Film-Transistor Liquid Crystal Display) display or an OLED (Organic Light Emitting Diode) display, or other appropriate display technology. The display interface 2356 may comprise appropriate circuitry for driving the display 2354 to present graphical and other information to a user. The control interface 2358 may receive commands from a user and convert them for submission to the processor 2352. In addition, an external interface 2362 may be provide in communication with processor 2352, so as to enable near area communication of device 2350 with other devices. External interface 2362 may provided, for example, for wired communication in some implementations, or for wireless communication in other implementations, and multiple interfaces may also be used.

The memory 2364 stores information within the computing device 2350. The memory 2364 can be implemented as one or more of a computer-readable medium or media, a volatile memory unit or units, or a non-volatile memory unit or units. Expansion memory 2374 may also be provided and connected to device 2350 through expansion interface 2372, which may include, for example, a SIMM (Single In Line Memory Module) card interface. Such expansion memory 2374 may provide extra storage space for device 2350, or may also store applications or other information for device 2350. Specifically, expansion memory 2374 may include instructions to carry out or supplement the processes described above, and may include secure information also. Thus, for example, expansion memory 2374 may be provide as a security module for device 2350, and may be programmed with instructions that permit secure use of device 2350. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 2364, expansion memory 2374, or memory on processor 2352 that may be received, for example, over transceiver 2368 or external interface 2362.

Device 2350 may communicate wirelessly through communication interface 2366, which may include digital signal processing circuitry where necessary. Communication interface 2366 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 2368. In addition, short-range communication may occur, such as using a Bluetooth, WiFi, or other such transceiver (not shown). In addition, GPS (Global Positioning System) receiver module 2370 may provide additional navigation- and location-related wireless data to device 2350, which may be used as appropriate by applications running on device 2350.

Device 2350 may also communicate audibly using audio codec 2360, which may receive spoken information from a user and convert it to usable digital information. Audio codec 2360 may likewise generate audible sound for a user, such as through a speaker, e.g., in a handset of device 2350. Such sound may include sound from voice telephone calls, may include recorded sound (e.g., voice messages, music files, etc.) and may also include sound generated by applications operating on device 2350.

The computing device 2350 may be implemented in a number of different forms, as shown in the figure. For example, it may be implemented as a cellular telephone 2380. It may also be implemented as part of a smartphone 2382, personal digital assistant, or other similar mobile device.

Additionally computing device 2300 or 2350 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

A number of embodiments of the invention have been described. Nevertheless, it will be understood that various modifications may be made without departing from the spirit and scope of the invention. Accordingly, other embodiments are within the scope of the following claims.

1.-135. (canceled)

136. A system for determining blood pressure measures for a subject, the system comprising:
a micro-motion sensor including a structure adapted to be applied against a surface of skin of the subject adjacent an artery with a constant hold-down force during a period of time during which a plurality of cardiac cycles occur, and the micro-motion sensor comprising a transducer to produce a continuous motion waveform representative of motion at the surface of the skin caused by pressure pulses propagating through the artery; and
processing equipment configured to:
(i) analyze a shape of a portion of the continuous motion waveform that corresponds to a single cardiac cycle of a single heartbeat, from among the plurality of cardiac cycles; and
(ii) calculate a blood pressure measurement for the single cardiac cycle of the single heartbeat based on the analysis of the shape of the portion of the continuous motion waveform that corresponds to the single cardiac cycle of the single heartbeat.

137. The system of claim 136, wherein the blood pressure measurement for the single cardiac cycle is one of a systolic blood pressure measurement for the single cardiac cycle and a diastolic blood pressure measurement for the single cardiac cycle.

138. The system of claim 136, wherein the processing equipment is further configured to calculate a blood pressure for multiple cardiac cycles based on:
(i) the analysis of the shape of the portion of the continuous motion waveform that corresponds to the single cardiac cycle, and
(ii) an analysis of a shape of a portion of the continuous motion waveform that corresponds to a preceding, single cardiac cycle.

139. The system of claim 138, wherein the blood pressure for the multiple cardiac cycles is one of average systolic blood pressure for the multiple cardiac cycles and average diastolic blood pressure for the multiple cardiac cycles.

140. The system of claim 136, wherein the processing equipment is further configured to identify the portion of the continuous motion waveform that corresponds to the single cardiac cycle.

141. The system of claim 140, wherein identifying the portion of the continuous motion waveform that corresponds to the single cardiac cycles includes:
(i) identifying a first instance of a pre-determined feature present in the continuous motion waveform, and
(ii) identifying a second instance of the pre-determined feature in the continuous motion waveform.

142. The system of claim 141, wherein the pre-determined feature is one of a systolic peak in the continuous motion waveform, a dicrotic notch in the continuous motion waveform, a local minimum immediately before a systolic rise to the systolic peak in the continuous motion waveform, and a local maximum that immediately follows the dicrotic notch in the continuous motion waveform.

143. The system of claim 141, wherein:
identifying the first instance of the pre-determined feature includes analyzing the continuous motion waveform for a local minimum or a local maximum; and
identifying the second instance of the pre-determined feature includes analyzing the continuous motion waveform for a local minimum or a local maximum.

144. The system of claim 136, wherein analyzing the shape of the portion of the continuous motion waveform that corresponds to the single cardiac cycle includes:
identifying locations of multiple pre-determined features within the portion of the continuous motion waveform that corresponds to the single cardiac cycle; and
determining a plurality of waveform measurements by analyzing relationships between the locations of the multiple pre-determined features, wherein calculating the blood pressure measurement for the single cardiac cycle is based on analysis of the plurality of waveform measurements that were determined by analyzing the relationships between the locations of the multiple pre-determined features.

145. The system of claim 144, wherein the multiple pre-determined features include one or more of:
(i) a systolic peak,
(ii) a dicrotic notch,
(ii) a local minimum immediately before a systolic rise to the systolic peak, and
(iv) a local maximum immediately after the dicrotic notch.

146. The system of claim 144, wherein the plurality of waveform measurements include one or more of:
(i) amplitude of a systolic peak,
(ii) width of the systolic peak,
(iii) area under the systolic peak,
(iv) width of a systolic upstroke to the systolic peak,
(v) area under the systolic upstroke to the systolic peak,
(vi) slope of the systolic upstroke to the systolic peak,
(vii) width of the systolic decline from the systolic peak,
(viii) area under the systolic decline from the systolic peak,
(ix) slope of the systolic decline from the systolic peak,
(x) depth of a dicrotic notch,
(xi) width of the dicrotic notch,
(xii) width of an entirety of the single cardiac cycle, and
(xiii) area under the entirety of the single cardiac cycle.

147. The system of claim 136, further comprising a display device, wherein the processing equipment is configured to interact with the display device to concurrently display:
(i) the portion of the continuous motion waveform that corresponds to the single cardiac cycle, or a blood pressure waveform generated therefrom; and
(ii) the blood pressure measurement for the single cardiac cycle.

148. The system of claim 147, wherein the concurrently display includes presenting information in real-time as the micro-motion sensor produces the continuous motion waveform, such that a presentation of (a) the portion of the continuous motion waveform, or the blood pressure waveform generated therefrom, and (b) the blood pressure measurement for the single cardiac cycle are replaced with a presentation of (a) a subsequent portion of the continuous motion waveform that corresponds to a subsequent, single cardiac cycle, or the blood pressure waveform generated therefrom, and (b) a subsequent blood pressure measurement for the subsequent, single cardiac cycle.

149. The system of claim 147, wherein the processing equipment is configured to interact with the display device to present the blood pressure measurement for the single cardiac cycle before the micro-motion sensor produces all of the continuous motion waveform for a subsequent, single cardiac cycle.

150. The system of claim 136, wherein the micro-motion sensor comprises an opto-electric sensor.

151. The system of claim 136, wherein the micro-motion sensor includes a fixation device that applies the structure of the micro-motion sensor to the surface of the skin, and the fixation device is structured so that application of the constant hold-down pressure maintains the structure of the micro-motion sensor in contact with the surface of the skin throughout the plurality of cardiac cycles without occluding the artery during the period of time during which the plurality of cardiac cycles occur.

152. The system of claim 151, wherein the fixation device is structured so that the constant hold-down pressure is less than about 20 mm Hg throughout the period of time during which the plurality of cardiac cycles occur.

153. The system of claim 151, wherein the fixation device is structured so that the constant hold-down pressure is in a range between about 5 mm Hg and 15 mmHg throughout the period of time during which the plurality of cardiac cycles occur.

154. The system of claim 151, wherein the fixation device comprises a spring that provides the constant hold-down pressure.

155. The system of claim 151, wherein the micro-motion sensor is structured to apply the constant hold-down force using the fixation device without activating an actuator that changes an amount of the hold-down force during the period of time during which the plurality of cardiac cycles occur.

156. The system of claim 136, wherein analyzing the shape of the portion of the continuous motion waveform that corresponds to the single cardiac cycle of the single heartbeat includes obtaining measurements for predefined shape parameters that specify characteristics of the shape of the portion of the continuous motion waveform.

157. The system of claim 156, wherein the predefined shape parameters and a process by which the blood pressure measurement is calculated for the single cardiac cycle is defined during a testing process during which one or more micro-motion sensors are applied to a variety of subjects to determine correspondence between measures of the shape parameters for single cardiac cycles and blood pressure measures for the respective single cardiac cycles.

158. The system of claim 136, wherein calculating the blood pressure measurement for the single cardiac cycle of the single heartbeat comprises comparing characteristics of the shape of the portion of the continuous motion waveform to stored characteristics that are pre-defined through analysis of shapes of single cardiac cycles and corresponding information that identifies respective blood pressure measurements for the shapes of the single cardiac cycles.

159. The system of claim 136, wherein the system further comprises a display component configured to display continuously updated blood pressure measures on a cycle-by-cycle basis.

160. The system of claim 159, wherein the display component is further configured such that the display component includes a representation of the continuous motion waveform and a blood pressure measure for each cardiac cycle of the continuous motion waveform presented by the display component.

161. A method of determining blood pressure measurements for a subject, the method comprising:
applying a structure of a micro-motion sensor against a surface of skin of the subject adjacent an artery with a constant hold-down force during a period of time during which a plurality of cardiac cycles corresponding to a respective plurality of heartbeats occur, the micro-motion sensor comprising a transducer to produce a continuous motion waveform representative of motion at the skin surface caused by pressure pulses propagating through the artery during the plurality of cardiac cycles;
analyzing a shape of a portion of the continuous motion waveform that corresponds to a single cardiac cycle of a single heartbeat, from among the plurality of cardiac cycles; and
calculating a blood pressure measurement for the single cardiac cycle of the single heartbeat based on the analysis of the shape of the portion of the continuous motion waveform that corresponds to the single cardiac cycle of the single heartbeat.

162. A micro-motion sensor device comprising:
An optical waveguide; and
A skin interface component comprising
(i) A button structure having a skin-facing surface for positioning against a skin surface adjacent an underlying blood vessel and an inner surface opposite the skin-facing surface positioned and configured to cause the optical waveguide to be flexed and/or compressed to modulate optical power propagating through the optical waveguide; and
(ii) A coil spring structure provided under an upper portion of the button structure and encompassing a lower portion of the button structure, wherein the coil spring structure is configured to bias the button structure outward in the direction of the skin-facing surface.

163. The micro-motion sensing device of claim 162, wherein the micro-motion sensor further comprises a housing having an opening formed therein; and the skin interface component is positioned to extend through the opening of the housing.
